(19) United States
(12) Patent Application Publication
(10) Pub. No.: US 2004/0175814 A1
(43) Pub. Date: Sep. 9, 2004

(54) Novel transferase and amylase, process for producing the enzymes, use thereof, and gene coding for the same

(75) Inventors: Masaru Kato, Takasaki-shi (JP); Yutaka Miura, Takasaki-shi (JP); Masako Kettoku, Takasaki-shi (JP); Akihiro Iwamatsu, Yokohama-shi (JP); Kazuo Kobayashi, Takasaki-shi (JP); Toshihiro Komeda, Yokohama-shi (JP)

Correspondence Address: FOLEY AND LARDNER, SUITE 500, 3000 K STREET NW, WASHINGTON, DC, 20007, US

(73) Assignee: KIRIN BEER KUBUSHIKI KAISHA

(21) Appl. No.: 10/688,276

(22) Filed: Oct. 20, 2003

Related U.S. Application Data
(63) Continuation of application No. 09/695,423, filed on Oct. 25, 2000, now abandoned.
Application No. 09/695,423 is a continuation of application No. 09/298,924, filed on Apr. 26, 1999, now Pat. No. 6,391,595.
Application No. 09/298,924 is a division of application No. 08/750,569, filed on Feb. 24, 1997.
Application No. 08/750,569 is a 371 of international application No. PCT/JP95/01189, filed on Jun. 14, 1995.

(30) Foreign Application Priority Data
Jun. 15, 1994 (JP) 6-133354
Aug. 18, 1994 (JP) 6-194223
Oct. 31, 1994 (JP) 6-290394
Nov. 21, 1994 (JP) 6-286917
Nov. 21, 1994 (JP) 6-311185
Apr. 21, 1995 (JP) 7-120673

(51) Int. Cl. C12N 9/10

(52) U.S. Cl. 435/193

(57) ABSTRACT

The invention provides a novel transferase that acts on a saccharide, as a substrate, composed of at least three sugar units wherein at least three glucose residues on the reducing end are linked α-1,4 so as to transfer the α-1,4 lingages to a α-1,α-1 linkages; a process for producing the transferase; a gene coding for the same; and a process for producing an oligosaccharide by using the same. Also provided are a novel amylase that has a principal activity of acting on a saccharide, as a substrate, composed of at least three sugar units wherein at least three sugar units on the reducing end side are glucose units and the linkage between the first and the second glucose units is α-1,α-1 while the linkage between the second and the third glucose units is α-1,4 so as to liberate α,α-trehalose by hydrolyzing the α-1,4 linkage and another activity of hydrolyzing the α-1,4 linkage within the molecular chain of the substrate and that liberates disaccharides and/or monosaccharides as the principal final products; a process for producing the amylase; a gene coding for the same; and a process for producing α,α-trehalose by using a combination of the transferase and the amylase.

### TECHNICAL FIELD

The present invention relates to:

a novel transferase, a process for producing the same, a process for producing an oligosaccharide by using the enzyme, a gene coding for the enzyme, and use thereof; and

a novel amylase, a process for producing the same, a process for producing α,α-trehalose by using the enzyme, a gene coding for the enzyme, and use thereof.

More specifically, as follows.

The present invention relates to a novel transferase which acts on a substrate saccharide, the substrate saccharide being composed of at least three sugar units wherein at least three glucose residues from the reducing end are α-1,4-linked, so as to transfer the α-1,4 linkages to α-1,α-1 linkages; and a process for producing the transferase. More particularly, the present invention relates to the above-mentioned enzyme produced from archaebacteria belonging to the order Sulfolobales, for example, bacteria of the genus Sulfolobus or Acidianus.

Further, the present invention relates to a novel process for producing trehaloseoligosaccharides or the like by using the above-mentioned novel enzyme, and more particularly, relates to an efficient and high-yield process for producing trehaloseoligosaccharides such as glucosyltrehalose and maltooligosyltrehaloses by using a maltooligosaccharide or the like as a raw material.

Moreover, the present invention relates to a DNA fragment coding for the above-mentioned novel transferase and to the use of the DNA fragment in genetic engineering.

The present invention relates to a novel amylase which acts on a substrate saccharide, the saccharide being composed of at least three sugar units wherein at least three sugar units from the reducing end are glucose residues, so as to liberate principally monosaccharides and/or disaccharides by hydrolyzing the substrate from the reducing end; and a process for producing the amylase. More particularly, the present invention relates to a novel amylase which has an principal activity of acting on a substrate saccharide, the substrate saccharide being composed of at least three sugar units wherein at least three sugar units from the reducing end side are glucose residues and the linkage between the first and the second glucose residues from the reducing end side is α-1,α-1 while the linkage between the second and the third glucose residues from the reducing end side is α-1,4, so as to liberate α,α-trehalose by hydrolyzing the α-1,4 linkage between the second and the third glucose residues; and a process for producing the amylase. The novel amylase also has another activity of endotype-hydrolyzing one or more α-1,4 linkages within the molecular chain of the substrate, and can be produced by bacteria belonging to the genus Sulfolobus. This enzyme is available for the starch sugar industry, textile industry, food industry, and the like.

Further, the present invention relates to a process for producing α,α-trehalose, characterized by using the above novel amylase in combination with the above novel transferase. In detail, the present invention relates to a process for producing α,α-trehalose in a high yield by using, as a raw material, any one of starch, starch hydrolysate and maltooligosaccharides, or a mixture of maltooligosaccharides, and as enzymes, the novel transferase and amylase of the present invention. Moreover, the present invention relates to a DNA fragment coding for the above novel amylase, and use of the DNA fragment in genetic engineering.

### BACKGROUND ART

I. Background Art of Transferase

Hitherto, in relation to glycosyltransferase acting on starch and starch hydrolysates such as maltooligosaccharides, various glucosyltransferases, cyclodextringlucano-transferases (CGTase), and others have been found [c.f. “Seibutsu-kagaku Jikken-hou” 25 (“Experimental Methods in Biochemistry”, Vol. 25), ‘Denpun. Kanren Toushitsu Kouso Jikken-hou’ (‘Experimental Methods in Enzymes for Starch and Relating Saccharides’), published by Gakkai-shuppan-sentah, Bioindustry, Vol. 9, No. 1 (1992), p. 39-44, and others]. These enzymes transfer a glucosyl group to the α-1,2, α-1,3, α-1,4, or α-1,6 linkage. However, an enzyme which transfers a glucosyl group to the α-1,α-1 linkage has not been found yet. Though trehalase has been found as an enzyme which acts on the α-1,α-1 linkage, trehalose is absolutely the only substrate for the enzyme, and the equilibrium or the reaction rate lies to the degrading reaction.

Recently, oligosaccharides were found to have physicochemical properties such as moisture-retaining ability, shape-retaining ability, viscous ability and browning-preventive ability, and bioactivities such as a low-calorigenetic property, an anticariogenic property and a bifidus-proliferation activity. In relation to that, various oligosaccharides such as maltooligosaccharides, branched-chain oligosaccharides, fructooligosaccharide, galacto-oligosaccharide, and xylooligosaccharide have been developed [c.f. “Kammiryo” (“Sweetener”)(1989), Medikaru-risahchi-sha (Medical Research Co.)(1989), Gekkan Fuhdokemikaru (Monthly Foodchemical)(1993), Feb. p. 21-29, and others].

Among oligosaccharides, the oligosaccharides which have no reducing end may include fructooligosaccharides having a structure composed of sucrose which is not reductive, and being produced by fructosyltransferase. Meanwhile, among starch hydrolysates such as maltooligosaccharides, the oligosaccharides which have no reducing end may include cyclodextrins produced by the above-mentioned CGTase, α,β-trehalose (neotrehalose), and reduced oligosaccharides chemically synthesized by hydrogenating the reducing end (oligosaccharide alcohol). These oligosaccharides having no reducing end have various physicochemical properties and bioactivities which are not possessed by conventional starch syrups and maltooligosac-charides. Accordingly, among maltooligosaccharides, the oligosaccharides the. reducing ends of which are modified with an α-1,α-1 linkage may be also expected to have the similar physicochemical properties and bioactivities to those possessed by the above-mentioned oligosaccharide having no reducing end, since such oligosaccharides also have no reducing end.

Here, the oligosaccharides the reducing ends of which are modified with an α-1,α-1 linkage as described above may be recognized as a trehaloseoligosaccharide in which α,α-trehalose is linked with glucose or a maltooligoshaccharide. Accordingly, such a trehaloseoligosaccharide may be expected to have the physicochemical properties and bioactivities which are possessed by the oligosaccharide having no reducing end, and in addition, may be expected to have the specific activities as exhibited by α,α-trehalose (c.f. Japanese Patent Laid-open Publication No. 63-500562).

Though it was reported that a trace amount of trehaloseoligosaccharides could be detected in yeast [Biosci. Biotech. Biochem., 57(7), p. 1220-1221 (1993)], this is the only report referring to its existence in nature. On the other hand, as to its synthesis by using an enzyme, though there has been a report of such synthesis [Abstracts of “1994 Nihon Nougei-kagaku Taikai” (“Annual Meeting of the Japan Society for Bioscience, Biotechnology and Agrochemistry in 1994”), p. 247], the method described in the report uses trehalose, which is expensive, as the raw material. Therefore, production at low cost has not yet been established.

Recently, Lama, et al. found that a cell extract from the Sulfolobus solfataricus strain MT-4 (DSM 5833), a species of archaebacteria, has a thermostable starch-hydrolyzing activity (Biotech. Forum. Eur. 8, 4, 2-1 (1991)). They further reported that the activity is also of producing trehalose and glucose from starch. The above-mentioned report, however, does not at all refer to the existence of trehaloseoligosaccharides such as glucosyltrehalose and maltooligosyltrehalose. Moreover, no investigation in archaebacteria other than the above-mentioned strain. has been attempted.

Meanwhile, an efficient process for obtaining the novel transferase should be established to efficiently produce trehaloseoligosaccharides.

Accordingly, mass-production of trehaloseoligosaccharides requires obtaining this novel transferase in a large amount. For achievement of this, it is preferable to obtain a gene coding for such transferase, and to produce the transferase in a genetic engineering manner. When such a gene can be obtained, it can be also expected, by using technologies of protein engineering, to obtain an enzyme having an improved thermostability, an improved pH stability, and an enhanced reaction rate. No report has, however, been made about gene cloning of such a gene yet.

An object of the present invention is to provide a novel transferase principally catalyzing the production of trehaloseoligosaccharides such as glucosyltrehalose and maltooligosyltrehaloses, and a process for producing the enzyme, and further, to provide a novel, efficient and high-yield process for producing principally trehalose-oligosaccharides such as glucosyltrehalose and maltooligosyltrehaloses by using such an enzyme from a raw material such as maltooligosaccharides.

Inventors earnestly investigated the trehalose-producing activity of archaebacteria and found that glucosyltrehalose can be produced from maltotriose as a substrate by cell extracts from various archaebacteria such as those belonging to the order Sulfolobales, and more specifically, the genera Sulfolobus, Acidianus, and others. Here, though production of trehalose and glucose was confirmed using an activity-measuring method described by Lama, et al. in which the substrate is starch, Inventors found that detection of trehaloseoligosaccha-rides such as glucosyltrehalose is extremely difficult. Also, Inventors found that the trehalose-producing activity as found by Lama, et al. disappears during the step for purification of cell extracts from archaebacteria. Consequently, the inventors recognized that the purification and characterization of the enzymes themselves which have such activities were substantially impossible.

Under such circumstances, Inventors made further investigations and conceived a novel activity-measuring method in which the substrate is a maltooligosaccharide such as maltotriose, and the index is activity of producing a trehaloseoligosaccharide such as glucosyl-trehalose. Then, it was found by a practice of the measuring method that a trehaloseoligosaccharide such as glucosyltrehalose can be easily detected. Further, the Inventor attempted to purify the enzyme having such activity from various bacterial strains, and found, surprisingly, that the enzyme thus obtained is quite a novel transferase which acts on maltotriose or a larger saccharide wherein at least three glucose residues from the reducing end are α-1,4-linked, and which transfers the linkage between the glucose residues at the reducing end into an α-1,α-1 linkage to produce trehaloseoligosaccha-rides such as glucosyltrehalose. Incidentally, the existence of trehaloseoligosaccharides which are produced from maltooligosaccharides or, the like by transferring the linkage between glucose residues at the reducing-end into an α-1,α-1 linkage was confirmed by ¹H-NMR and ¹³C-NMR (c.f. Examples I-1, 7 and 8).

Inventors further found that such a novel enzyme is available for producing a large amount of trehaloseoligosaccharides, for example, glucosyltrehalose and maltooligosyltrehalose from saccharides such as maltooligosaccharides, and have accomplished the present invention.

Moreover, Inventors isolated the genes coding for such a novel enzyme, and have now established a process for producing the novel transferase by using such genes in a genetic engineering manner.

II. Background Art of Amylase

“Amylase” is a generic term for the enzymes which hydrolyze starch. Among them, α-amylase is an enzyme which endotype-hydrolyzes an α-1,4 glucoside linkage. Alpha-amylase widely exists in the living world. In mammals, α-amylase can be found in saliva and pancreatic fluid. In plants, malt has the enzyme in large amounts. Further, α-amylase widely exists in microorganisms. Among them, α-amylase or the like which is produced by some fungi belonging to the genus Aspergillus or some bacteria belonging to the genus Bacillus is utilized in the industrial fields [“Amirahze” (“Amylase”), edited by Michinori Nakamura, published by Gakkai-shuppan-sentah, 1986].

Such α-amylase is industrially-and widely used for various purposes, for example, for starch-liquefying processes in starch sugar industries, and for desizing processes in textile industries, and therefore, the enzyme is very important from an industrial view. The following are listed as important conditions for the starch-liquefying process in “Kouso-Ouyou no Chishiki” (written by Toshiaki Komaki, published by Sachi-Shobou, 1986): 1) the starch molecules should be liquefied as completely as possible, 2) the products produced by the liquefaction are favorable for the purpose of the subsequent saccharifying process, 3) the condition does not cause retrogradation of the products by the liquefaction, and 4) the process should be carried out in a high concentration as much as possible (30-35%) in view of reducing cost. A starch-liquefying process may be performed, for example, by a continuous liquefaction method at a constant temperature, or by the Jet-Cooker method. Ordinarily, a thick starch-emulsion containing α-amylase is instantaneously heated to a high temperature (85-110° C.), and then the α-amylase is put into action to perform liquefaction at the same time as starch begins to be gelatinized and swollen. In other words, the starch-liquefying process requires a temperature sufficient to cause the starch to swell before the enzyme can act. Enzymes capable of being used in such fields are, for example, the above-mentioned thermostable α-amylases produced by fungi of the Aspergillus oryzae group belonging to the genus Aspergillus or bacteria belonging to the genus Bacillus. In some cases, the addition of calcium is required for further improving thermostability of these enzymes. In the starch-liquefying process, once the temperature declines while the α-amylase has not yet acted on the starch-micelles which are swelled and going to be cleaved, starch will be agglutinated again to form new micelles (insoluble starch) which are rarely liquefied by α-amylase. As a result, the liquid sugar thus produced will be turbid and hard to filtrate, as is a known problem. Some methods which increase the liquefaction degree, i.e. dextrose equivalent (DE), are used in order to prevent such an event. However, in some cases, such as an enzymatic production of maltose, DE should be maintained as low as possible, namely, the polymerization degree of the sugar chain should be maintained to a high degree in order to keep a high yield. Accordingly, when an enzyme is further used for a process subsequent to a starch-liquefying process, use of an enzyme thermostable enough for use in a series of high temperatures will allow the progress of the reaction without producing slightly soluble starch even by using a high concentration of starch, and at the same time, such use will be advantageous in view of process control and sanitary control because the risk of contamination with microorganisms can be decreased. Meanwhile, when the enzyme is immobilized in a bioreactor to use the enzyme recyclically, it is believed to be important that the enzyme has high stability, and especially high thermostability, since the enzyme may be exposed to a relatively high temperature during immobilization. If the enzyme has a low thermostability, it will possibly be inactivated during the immobilization procedure. As is obvious from the above, an enzyme having a high thermostability can be used very advantageously in several industrial fields, for example, a starch-liquefying process, and such an enzyme is desired.

In addition, screening of thermophilic and hyperthermophilic bacteria has been widely carried out in recent years in order to obtain thermostable enzymes including amylase. Archaebacteria belonging to the order Thermococcales and the genus Pyrococcus are also the objects of screening, and were reported to produce α-amylase [Applied and Environmental Microbiology, pp.1985-1991, (1990); Japanese Patent Laid-open Publication No. 6-62869; and others]. Additionally, archaebacteria belonging to the genus Sulfolobus are the objects of screening, and isolation of thermostable enzymes was reported. Here, archaebacteria belonging to the genus Sulfolobus are taxonomically defined by the following characteristics:

being highly thermophilic: being possible to grow in a temperature range of 55° C. -88° C.;

being acidophilic: being possible to grow in a pH range of 1-6;

being aerobic; and

being sulfur bacteria: being cocci having irregular form, and a diameter of 0.6-2 μm. Accordingly, if an archaebacterium belonging to the genus Sulfolobus produces an amylase, the amylase is expected to be also thermo-stable. Lama, et al.found that a thermostable starch-hydrolyzing activity exists in a cell extract from the Sulfolobus solfataricus strain MT-4 (DSM 5833) [Biotech. Forum. Eur. 8, 4, 2-1 (1991)]. This article reported that α,α-trehalose and glucose can be produced from starch by this activity. However, purification of the active substance was performed only partially, and the true substance exhibiting the activity has not yet been identified. In addition, the enzymatic characteristics of the activity has not been clarified at all. The Inventors' investigations, the details of which will be described below, revealed that the active substance derived from the above-mentioned bacterial strain and allowed to act on starch by Lama, et al. was a mixture containing a plurality of enzymes, and that α,α-trehalose and glucose are the final products obtained by using the mixture.

As another characteristic, α-amylase has an activity of, at an initial stage, decreasing the quantity of iodo-starch reaction, namely, an activity of endotype-hydrolyzing α-1,4-glucan (liquefying activity). There are several modes in the reaction mechanism of such liquefying-type amylase. In other words, it is known that each amylase has common characteristics in view of endotype-hydrolyzing activity but has individual characteristics in view of patterns for hydrolyzing maltooligosaccharides. For example, some recognize a specific site for hydrolysis of the substrate from the non-reducing end, and others recognize a specific site for hydrolysis of the substrate from the reducing end. Further, some hydrolyze the substrate to principally produce glucose; others to principally produce maltose or maltooligosaccharides. More specifically, the α-amylase derived from pancreas hydrolyzes the α-1,4 linkage second or third from the reducing end [“Denpun.Kanren Toushitsu Kouso Jikken-hou” (“Experimental methods in enzymes for starch and relating saccharides”), written by Michinori Nakamura and Keiji Kainuma, published by Gakkai-Shuppan-Sentah, 1989]. The α-amylase derived from Bacillus subtilis hydrolyzes the α-1,4 linkage sixth from the non-reducing end or third from the reducing end [“Kouso-Ouyou no Chishiki” (“Knowledge in Application of Enzymes”), written by Toshiaki Komaki, published by Sachi-Shobou, 1986]. It is believed that such a difference between the reaction modes of α-amylases can be attributed to the structure of each enzyme, and the “Subsite theory” is proposed for explanation of these events. Additionally, the existence of an α-amylase having transferring activities or condensation activities has been confirmed. Further, a particular α-amylase which produces a cyclodextrin has been found.

On the other hand, α,α-trehalose consists of two glucose molecules which are α-1,α-1-linked together at the reducing group of each molecule. It is known that α,α-trehalose exists in many living things, plants and microorganisms of the natural world, and has many function such as preventing the biomembrane from freezing or drying, and being an energy source in insects. Recently, α,α-trehalose was evaluated in the fields of medicine, cosmetics and food as a protein stabilizer against freezing and drying (Japanese Examined Patent Publication No. 5-81232, Japanese Patent Laid-open Publication No. 63-500562, and others). However, α,α-trehalose is not often used practically. This may be because no mass-productive process has been established yet.

Examples of the conventional process for producing α,α-trehalose are as follows:

A process comprising extraction from an yeast (Japanese Patent Laid-open Publications Nos. 5-91890 and 4-360692, and others);

a process comprising intracellular production by an yeast (Japanese Patent Laid-open Publication No. 5-292986, European Patent No. 0451896, and others); and

a process comprising production by a microorganism belonging to the genus Sclerotium or the genus Rhizoctonia (Japanese Patent Laid-open Publication No. 3-130084). However, these processes, as comprising intracellular production, require a purification process comprising multiple steps for spallation of bacterial bodies and removal of debris. Meanwhile, several investigations were made into extracellular production by a fermentation using a microorganism, for example, a microorganism belonging to the genus Arthrobacter (Suzuki T, et al., Agric. Biol. Chem., 33, No. 2, 190, 1969) or the genus Nocardia (Japanese Patent Laid-open Publication No. 50-154485), and glutamate-producing bacteria (French Patent No. 2671099, Japanese Patent Laid-open Publication No. 5-211882, and others). Further, production by a gene encoding an enzyme for α,α-trehalose metabolism was attempted (PCT Patent No. 93-17093). Any of the above processes use glucose or the like as the sugar source, and utilize a metabolic system which requires ATP and/or UTP as the energy source. These processes, therefore, require a complicated purification process to obtain α,α-trehalose from the culture medium. Moreover, some investigations were attempted into production by an enzymatic process using, for example, trehalose phosphorylase (Japanese Examined Patent Publication No. 63-60998), or trehalase (Japanese Patent Laid-open Publication No. 7-51063). These processes, however, have some problems in mass-production of the enzymes, stability of the enzymes, and others. All of the processes of the prior art as described above. have problems such as a low yield, complexity in the purification process, low production, and complexity in preparation of the enzyme. Therefore, a process having industrial applicability has not been established yet. Under. the circumstances, a process for more efficiently producing α,α-trehalose is strongly desired to be established.

As described above, α,α-trehalose was found widely in nature, and the existence of it in archaebacteria was also confirmed (System. Appl. Microbiol. 10, 215, 1988). Specifically, as mentioned above, Lama, et al. found that a thermostable starch-hydrolyzing activity exists in a cell extract from an archaebacterium species, the Sulfolobus solfataricus strain MT-4 (DSM 5833), and confirmed the existence of α,α-trehalose in the hydrolyzed product [Biotech. Forum. Eur. 8, 4, 2-1 (1991), cited before]. This article reported that the activity was of producing α,α-trehalose and glucose from starch. The article, however, actually reported only an example in which the substrate was 0.33% soluble starch, the amount of α,α-trehalose produced thereby was extremely small, and besides, the ratio of produced α,α-trehalose to produced glucose was 1:2. Accordingly, an isolation process is necessary to remove glucose which is produced in a large amount as a by-product, and the purpose of establishing a process for mass-producing α,α-trehalose cannot be achieved at all.

Inventors, as described above, found that an archaebacteria belonging to the order Sulfolobales produce a transferase which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage. Further, Inventors invented a process for producing trehaloseoligosaccharides such as glucosyltrehalose and maltooligosyltrehaloses from maltooligosaccharides by using this enzyme. Here, the trehaloseoligosaccharide is a maltooligosaccharide the reducing end side of which is modified with an α-1,α-1 linkage.

In the meantime, no report has been made, as far as Inventors know, as to an formerly-known enzyme capable of acting on a trehaloseoligosaccharide which is derived from a maltooligosaccharide by transforming the first linkage from the reducing end into an α-1,α-1 linkage, and capable of hydrolyzing specifically the α-1,4 linkage next to the α-1,α-1 linkage to liberate α,α-trehalose in a high yield. In other words, conventional amylase cannot hydrolyze trehaloseoligosaccharide specifically at the α-1,4 linkage between the second and third glucose residues from the reducing end side to liberate α,α-trehalose. It will, therefore, markedly benefit the mass-production of α,α-trehalose if an amylase can be developed, such amylase being capable of catalyzing the reaction for producing α,α-trehalose as well as hydrolyzing the α-1,4 linkage in the molecular chain of starch or starch hydrolysate.

In addition, mass-production of α,α-trehalose requires obtaining the novel amylase in a large amount. For this purpose, it is preferable to obtain a gene coding for the amylase and to produce the enzyme in a genetic engineering manner. Further, if such a gene can be obtained, it can also be expected to obtain, by using a technology of protein engineering, an enzyme which has improved thermostability, improved pH stability, and an enhanced reaction rate.

An object of the present invention is to provide a novel amylase which has an activity of endotype-hydrolyzing the α-1,4 linkage in the molecular chain of starch or starch hydrolysate, and which can catalyze the reaction of liberating α,α-trehalose, wherein the enzyme acts on a trehaloseoligosaccharide which is derived from a maltooligosaccharide by transforming the first linkage from the reducing end into an α-1,α-1 linkage, and hydrolyzes specifically the α-1,4 linkage between the second and third glucose. residues from the reducing end side, and is to provide a process for producing such an enzyme. Another object of the present invention is to provide a novel process for efficiently producing α,α-trehalose in a high yield from a low-cost raw material such as starch, starch hydrolysate, and maltooligosaccharides by using the enzyme.

Inventors energetically investigated starch-hydrolyzing activity derived from archaebacteria. As a result, Inventors found that a thermostable starch-hydrolyzing activity exists in cell extracts from various archaebacteria belonging to the order Sulfolobales, and more specifically, the genus Sulfolobus. The saccharides produced.by hydrolysis of starch were found to be glucose and α,α-trehalose, similar to the description in the article by Lama, et al. Inventors then examined extracts from various bacterial strains for characteristics of the starch-hydrolyzing activity. As a result, Inventors found that the enzymes produced by those strains are mixtures of enzymes comprising various endotype or exotype amylases such as liquefying amylase and glucoamylase, and transferase, in view of enzymatic activity such as starch-hydrolyzing activity and α,α-trehalose-producing activity. In addition, such enzymatic activities were found to be attributed to synergism by activities of these mixed enzymes. Further, when the activity-measuring method proposed by Lama, et al. is employed in purification of each enzyme, in which the index is decrement of blue color derived from iodo-starch reaction, the purification of each enzyme having such an activity resulted in a low yield on the whole, and such purification procedure was found to be very difficult. These events may be attributed to low sensitivity and low quantifying ability of the activity-measuring method. Moreover, the Inventors' strict examination revealed that purification and isolation could not be accomplished at all, in terms of protein, by the partial-purification method described in the article by Lama, et al.

Under such circumstances, Inventors have made further investigation, and conceived a new activity-measuring method in which the substrate is a trehaloseoligosaccharide such as maltotriosyltrehalose, and the index is activity of liberating α,α-trehalose. By a practice of this measuring method, it was revealed that amylase activity can be easily detected using such a method. Inventors then tried to achieve purification of the enzyme having such an activity in various bacterial strains, and finally, succeeded in purification and isolation of such an amylase. Further, Inventors examined enzymatic characteristics of the isolated and purified amylase, and found, surprisingly, that the enzyme thus obtained has a novel action mechanism, namely, has the following characteristics together:

The enzyme exhibits an activity of endotype-hydrolyzing starch or starch hydrolysate;

the enzyme exhibits an activity of hydrolyzing starch hydrolysate, a maltooligosaccharide or the like from the reducing end to produce monosaccharides and/or disaccharides;

the enzyme exhibits a higher reactivity to a saccharide which is composed of at least three sugar units wherein the linkage between the first and second glucose residues from the reducing end side is α-1,α-1, and the linkage between the second and third glucose residues from the same end side is α-1,4 (for example, trehaloseoligosaccharides), as compared with the reactivity to each of the corresponding maltooligosaccharides; and

the enzyme has an activity of acting on such substrate saccharides composed of at least three sugar units so as to liberate α,α-trehalose by hydrolyzing the α-1,4 linkage between the second and third glucose residues from the reducing end side.

Moreover, Inventors isolated a gene coding for such novel enzyme, and now, have established a process for producing, in a genetic engineering manner, a recombinant novel amylase by utilizing such a gene.

### DISCLOSURE OF INVENTION

I. Novel Transferase

The present invention provides a novel transferase (hereinafter referred to as “novel transferase of the present invention”, or simply referred to as “the enzyme of the present invention” or “the present enzyme”) which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage.

In another aspect, the present invention provides a novel transferase which acts on a substrate maltooligosaccharide, all of the constituting glucose residues of the maltooligosaccharide being α-1,4-linked, so as to transfer the first α-1,4 linkage from the reducing end into an α-1,α-1 linkage.

Further, the present invention provides a process for producing the novel transferase of the present invention, wherein a bacterium capable of producing a transferase having such activities is cultivated in a culture medium, and the transferase is isolated and purified from the culture on the basis of an activity-measuring method in which the substrate is a maltooligosaccharide, and the index is the activity of producing trehaloseoligosaccharides.

Moreover, the present invention provides a process for producing a saccharide having an end composed of a couple of α-1,α-1-linked sugar units, characterized in that the enzyme of the present invention is used and allowed to act on a substrate saccharide, the substrate saccharide being composed of at least three sugar units wherein at least three glucose residues from the reducing end are α-1,4-linked, so as to produce the objective saccharide in which at least three sugar units from the reducing end side are glucose residues and the linkage between the first and second glucose residues from the reducing end side is α-1,α-1 while the linkage between the second and third glucose residues from the reducing end side is α-1,4.

Furthermore, the present invention provides a process for producing a trehaloseoligosaccharide, wherein the enzyme of the present invention is used, and the substrate is each of maltooligosaccharides or a mixture thereof.

Additionally, an object of the present invention is to provide a gene coding for the transferase.

Further, another object of the present invention is to provide a recombinant novel transferase and a process for producing the same by using the above-mentioned gene.

Moreover, an object of the present invention is to provide an efficient process for producing trehaloseoligosaccharides such as glucosyltrehalose and maltoglucosyltrehalose by using a recombinant novel transferase.

Accordingly, the DNA fragment based on the present invention comprises a gene coding for a novel transferase which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage.

Further, the recombinant novel transferase according to the present invention is the product achieved by expression of the above-mentioned DNA fragment.

Moreover, the process for producing a recombinant novel transferase according to the present invention comprises:

culturing a host cell transformed with the above-mentioned gene;

producing said recombinant novel transferase in the culture; and

collecting the products.

II. Novel Amylase

The present invention provides a novel amylase which acts on a substrate saccharide, the substrate saccharide being composed of at least three sugar units wherein at least three sugar units from the reducing end are glucose residues, so as to liberate principally monosaccharides and/or disaccharides by hydrolyzing the substrate from the reducing end side.

In another aspect, the present invention provides a novel amylase which has a principal activity of acting on a substrate saccharide, the substrate saccharide being composed of at least three sugar units wherein at least three sugar units from the reducing end side are glucose residues and the linkage between the first and the second glucose residues from the reducing end side is α-1,α-1 while the linkage between the second and the third glucose residues from the reducing end side is α-1,4, so as to liberate α,α-trehalose by hydrolyzing the α-1,4 linkage between the second and the third glucose residues.

Further, in another aspect, the present invention provides a novel amylase which also has an activity of endotype-hydrolyzing one or more α-1,4 linkages in the molecular chain of the substrate as well as the above-described activity.

Moreover, the present invention provides a process for producing aforementioned amylase, wherein a bacterium capable of producing the above amylase of the present invention is cultivated in a culture medium, and then the amylase is isolated and purified from the culture on the basis of an activity-measuring method; in which the substrate is a trehaloseoligosaccharide, and the index is the activity of producing α,α-trehalose.

Inventors allowed the above amylase of the present invention in combination with the aforementioned transferase of the present invention to act on a glucide raw material such as starch, starch hydrolysate, and maltooligosaccharides, and found that α,α-trehalose can be efficiently produced thereby with a high yield.

Accordingly, the present invention also provides a process for producing α,α-trehalose, wherein the above amylase and transferase of the present invention are used in combination.

Additionally, an object of the present invention is to provide a novel amylase and a gene coding for the same.

Further, another object of the present invention is to provide a recombinant novel amylase and a process for producing the same by using the aforementioned gene.

Moreover, another object of the present invention is to provide a process for producing α,α-trehalose by using a recombinant novel amylase.

Therefore, the gene coding for the amylase according to the present invention comprises a DNA sequence coding for a novel amylase which has the following activities:

(1) An activity of endotype-hydrolyzing an α-1,4 glucoside linkage in a sugar chain;

(2) an activity of acting on a substrate saccharide, the substrate saccharide being composed of at least three sugar units wherein at least three sugar units from the reducing end are α-1,4-linked glucose residues, so as to liberate principally monosaccharides and/or disaccharides by hydrolyzing the substrate from the reducing end side; and

(3) a principal activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues.

Further, the recombinant novel amylase according to the present invention is a product achieved by expression of the above-described gene.

Furthermore, the process for producing α,α-trehalose according to the present invention comprises a step to put the above-described recombinant novel amylase and a novel transferase into contact with a saccharide of which at least three glucose residues from the reducing end are α-1,4-linked, wherein the transferase can act on a substrate saccharide, the substrate saccharide being composed of at least three sugar units wherein at least three glucose residues from the reducing end are α-1,4-linked, so as to transfer the first α-1,4-linkage from the reducing end into an α-1,α-1 linkage.

### BRIEF DESCRIPTION OF DRAWINGS

FIG. 1 is a graph showing the results of an analysis by TSK-gel Amide-80 HPLC, performed on the product which is obtained in Example I-1 by using the cell extract derived from the Sulfolobus solfataricus strain KM1.

FIG. 2 is a graph showing thermostability of the present transferase which is obtained in Example I-2 from the Sulfolobus solfataricus strain KM1.

FIG. 3 is a graph showing pH stability of the present transferase which is obtained in Example I-2 from the Sulfolobus solfataricus strain KM1.

FIG. 4 is a graph showing reactivity of the present transferase which is obtained in Example I-2 from the Sulfolobus solfataricus strain KM1, when examined at each temperature.

FIG. 5 is a graph showing optimum pH for reaction of the present transferase which is obtained in Example I-2 from the Sulfolobus solfataricus strain KM1.

FIG. 6 is a graph showing patterns of reaction products derived from maltotriose by using the present transferase which is obtained in Example I-2 from the Sulfolobus solfataricus strain KM1.

FIG. 7 is a graph showing patterns of reaction products derived from maltotetraose by using the present transferase which is obtained in Example I-2 from the Sulfolobus solfataricus strain KM1.

FIG. 8 is a graph showing patterns of reaction products derived from maltopentaose by using the present transferase which is obtained in Example 1-2 from the Sulfolobus solfataricus strain KM1.

FIG. 9 is a graph showing the results of an analysis by AMINEX HPX-42A HPLC, performed on the reaction product derived from a mixture of maltooligosaccharides by using the present transferase which is obtained in Example I-2 from the Sulfolobus solfataricus strain KM1.

FIG. 10 is a graph showing the results of an analysis by TSK-gel Amide-80 HPLC, performed on the reaction product derived from maltotriosyltrehalose subjected to reaction with the crude enzyme solution which is obtained in Example II-1 from the Sulfolobus solfataricus strain KM1.

FIG. 11 is a graph showing the results of an analysis by AMINEX HPX-42A HPLC, performed on the reaction product derived from soluble starch subjected to reaction with the crude enzyme solution which is obtained in Example II-1 from the Sulfolobus solfataricus strain KM1.

FIG. 12 is a graph showing thermostability of the present amylase which is obtained in Example II-2 from the Sulfolobus solfataricus strain KM1.

FIG. 13 is a graph showing pH stability of the present amylase which is obtained in Example II-2 from the Sulfolobus solfataricus strain KM1.

FIG. 14 is a graph showing reactivity of the present amylase which is obtained in Example II-2 from the Sulfolobus solfataricus strain KM1, examined at each reaction temperature.

FIG. 15 is a graph showing optimum pH for reaction of the present amylase which is obtained in Example II-2 from the Sulfolobus solfataricus strain KM1.

FIG. 16 is a graph showing reactivity of the present amylase to various substrates, the amylase being obtained in Example II-2 from the Sulfolobus solfataricus strain KM1.

FIG. 17 contains graphs showing the results of analyses by AMINEX HPX-42A HPLC, performed on the reaction products derived from maltopentaose, Amylose DP-17, and soluble starch, respectively, subjected to reaction with the present amylase which is obtained in Example II-2 from the Sulfolobus solfataricus strain KM1.

FIG. 18 is a graph showing the results of an analysis by TSK-gel Amide-80 HPLC, performed on the reaction product derived from maltotriosyltrehalose subjected to reaction with the present amylase which is obtained in Example II-2 from the Sulfolobus solfataricus strain KM1.

FIG. 19 is a graph showing the results of an analysis by TSK-gel Amide-80 HPLC, performed on the reaction product derived from maltopentaosyltrehalose subjected to reaction with the present amylase which is obtained in Example II-2 from the Sulfolobus solfataricus strain KM1.

FIG. 20 is a graph showing time-course changes in disappearance of color generated by iodo, and starch-hydrolyzing percentage when the present amylase which is obtained in Example II-2 from the Sulfolobus solfataricus strain KM1 is made to act on soluble starch.

FIG. 21 is a graph showing time-course change in radioactivity of the reaction product derived from radiolabeled maltopentaose subjected to reaction with the present amylase which is obtained in Example II-2 from the Sulfolobus solfataricus strain KM1.

FIG. 22 is a graph showing time-course change in radioactivity of the reaction product derived from radiolabeled maltotriosyltrehalose subjected to reaction with the present amylase which is obtained in Example II-2 from the Sulfolobus solfataricus strain KM1.

FIG. 23 is a graph showing reactivity of α-amylase derived from porcine pancreas to various substrates.

FIG. 24 is a graph showing the results of an analysis by TSK-gel Amide-80 HPLC, performed on the reaction product derived from maltopentaosyltrehalose subjected to reaction with α-amylase which is derived from porcine pancreas.

FIG. 25 is a graph showing the results of an analysis by AMINEX HPX-42A HPLC, performed on the reaction product derived from soluble starch subjected to reaction with transferase and the present amylase which is obtained in Example II-2 from the Sulfolobus solfataricus strain KM1.

FIG. 26 is an illustration showing the restriction map of each insertional fragment pKT1, pKT11 or pKT21, containing a gene which codes for the novel transferase, and is obtained in Example I-12 from the Sulfolobus solfataricus strain KM1.

FIG. 27 is an illustration showing a process for constructing the plasmid pKT22.

FIG. 28 is a graph showing the results of an analysis by TSK-gel Amide-80 HPLC, performed on the product derived from maltotriose by using the recombinant novel transferase.

FIG. 29 is an illustration showing the restriction map of the insertional fragment pO9T1 containing a gene which codes for the novel transferase, and-is obtained in Example I-16 from the Sulfolobus acidocaldarius strain ATCC 33909.

FIG. 30 is an illustration showing a process for constructing the plasmid pO9T1.

FIG. 31 is an illustration showing the homology between the amino acid sequence of the novel transferase derived from the Sulfolobus solfataricus strain KM1 and that derived from the Sulfolobus acidocaldarius strain ATCC 33909.

FIG. 32 is an illustration showing the homology between the base sequence of the gene coding for the novel transferase derived from the Sulfolobus solfataricus strain KM1 and that derived from the Sulfolobus acidocaldarius strain ATCC 33909.

FIG. 33 is a graph showing the results of an analysis by AMINEX HPX-42A HPLC, performed on the product derived from a maltooligosaccharide mixture by using the recombinant novel transferase.

FIG. 34 is an illustration showing the restriction map of the insertional fragment pKA1 containing a gene which codes for the novel amylase, and is derived from the Sulfolobus solfataricus strain KM1.

FIG. 35 is an illustration showing the restriction map of pKA2.

FIG. 36(A) is a graph showing the results of an analysis performed on the product derived from a maltotriosyltrehalose by using the recombinant novel amylase according to the present invention; and FIG. 36(B) is a graph showing the results of an analysis performed on the product derived from soluble starch by using the recombinant novel amylase according to the present invention.

FIG. 37 is a graph showing time-course changes in disappearance of color generated by iodo, and starch-hydrolyzing percentage when the recombinant novel amylase according to the present invention is made to act on soluble starch.

FIG. 38 is an illustration showing the restriction map of the insertional fragment pO9A1 containing a gene which codes for the novel amylase, and is derived from the Sulfolobus acidocaldarius strain ATCC 33909.

FIG. 39 is an illustration showing the process for producing pO9A1 from pO9A2.

FIG. 40 is an illustration showing the homology between the amino acid sequence of the novel amylase derived from the Sulfolobus acidocaldarius strain ATCC 33909 and that derived from the Sulfolobus solfataricus strain KM1.

FIG. 41 is an illustration showing the homology between the base sequence of the gene coding for the novel amylase derived from the Sulfolobus acidocaldarius strain ATCC 33909 and that derived from the Sulfolobus solfataricus strain KM1.

FIG. 42 is a graph showing the results of an analysis performed on the product derived from 10% soluble starch subjected to reaction with the recombinant novel amylase which is obtained in Example II-19, and the recombinant novel transferase which is obtained in Example I-20.

### BEST MODE FOR CARRYING OUT THE INVENTION

Deposit of Microorganisms

The below-mentioned novel bacterial strain KM1, which was substantially purely isolated from nature by the Inventor, was deposited in the National Research Institutes, the Life Science Laboratory for Industry on Apr. 1, 1994 as acceptance No. FERM BP-4626.

The Escherichia coli strain JM109/pKT22 transformed with the plasmid pKT22 according to the present invention (c.f. below-described Example I-14), and the Escherichia coli strain JM109/pO9T1 transformed with the plasmid pO9T1 (c.f. below-described Example I-16), which contain the gene coding for the novel transferase according to the present invention, were deposited in the National Research Institutes, the Life Science Laboratory for Industry on Oct. 21, 1994 as acceptance No. FERM BP-4843 and on May 9, 1995 as the acceptance No. FERM BP-5093, respectively.

Further, the Escherichia coli strain JM109/pKA2 transformed with the plasmid pKA2 according to the present invention (c.f. below-described Example II-19), and the Escherichia coli strain JM109/pO9A1 transformed with the plasmid pO9A1 (c.f. below-described Example II-22), which contain the gene coding for the novel amylase according to the present invention, were deposited in the National Research Institutes, the Life Science Laboratory for Industry on Oct. 31, 1994 as acceptance No. FERM BP-4857 and on May 9, 1995 as acceptance No. FERM BP-5092, respectively.

I. Novel Transferase

Microorganisms Producing the Novel Transferase of the Present Invention

The archaebacteria which can be used in the present invention may include the Sulfolobus solfataricus strain ATCC 35091 (DSM 1616), the Sulfolobus solfataricus strain DSM 5833, the Sulfolobus solfataricus strain KM1 (the below-described novel bacterial strain which was substantially purely isolated from nature by Inventors), the Sulfolobus acidocaldarius strain ATCC 33909 (DSM 639), and the Acidianus brierleyi strain DSM 1651.

As described above, a fairly wide variety of archaebacteria taxonomically classified under the order Sulfolobales, to which the genera Sulfolobus and Acidianus belong, may be considered as the microorganisms which can produce the novel transferase of the present invention. Here, the archaebacterium belonging to the order Sulfolobales are taxonomically defined as being highly acidophilic and thermophilic, being aerobic, and being sulfur bacteria (coccal bacteria). The aforementioned Acidianus brierleyi strain DSM 1651, which belongs to the genus Acidianus, had been formerly classified as Sulfolobus brierleyi strain DSM 1651, and the aforementioned Sulfolobus solfataricus strain DSM 5833 had been named as Caldariella acidophila. From these facts, microorganisms which are closely related to the above-described archaebacteria genetically or taxonomically and which are capable of producing the enzyme of the same kind can be used in the present invention.

Sulfolobus solfataricus Strain KM1

Among the above-illustrated microorganisms, the Sulfolobus solfataricus strain KM1 is the bacterial strain which Inventors isolated from a hot spring in Gunma Prefecture, and which exhibits the following characteristics.

(1) Morphological Characteristics

The shape and size of the bacterium: Coccoid (no regular form), and a diameter of 0.6-2 μm.

(2) Optimum Growth Conditions

pH: Capable of growing in pH of 3-5.5, and optimally, in pH of 3.5-4.5.

Temperature: Capable of growing in a temperature range of 55° C. -85° C., and optimally in a temperature range of 75° C. -80° C.

Capable of metabolize sulfur.

(3) Classification in view of aerobic or anaerobic: aerobic.

According to the above characteristics, identification of the bacterial strain was carried out on the basis of Bergey's Manual of Systematic Bacteriology Volume,3 (1989). As a result, the strain was found to be one of Sulfolobus solfataricus, and thus named as Sulfolobus solfataricus strain KM1.

In culturing the above bacterial strain, the culture medium to be used may be either liquid or solid, and ordinarily, a concussion culturing or a culturing with aeration and stirring is performed using a liquid culture medium. In other words, the culture medium to be used is not limited as long as it is suitable for the bacterial growth, and the suitable examples of such culture media may include the Sulfolobus solfataricus Medium which is described in Catalogue of Bacteria and Pharges 18th edition (1992) published by American Type Culture Collection (ATCC), and in Catalogue of Strains 5th edition (1993) published by Deutsche Sammlung von Mikroorganismen und Zellkulturen GmbH (DSM). Starch, maltooligosaccharide and/or the like may be further added as a sugar source. Moreover, the culturing conditions are also not limited as long as they are based on the above-described growable temperature and pH.

Cultivation of the Microorganisms which Produce the Novel Transferase of the Present Invention

The culturing conditions for producing the novel transferase of the present invention may suitably be selected within ranges in which the objective transferase can be produced. When a concussion culturing or a culturing with aeration and stirring using a liquid medium is employed, the culturing for 2-7 days should suitably be performed at a pH and a temperature which allow the growth of each microorganism. The culture medium to be suitably used is, for example, the Sulfolobus solfataricus Medium which is described in Catalogue of Bacteria and Pharges 18th edition (1992) published by American Type Culture Collection (ATCC), and in Catalogue of Strains 5th edition (1993) published by Deutsche Sammlung von Mikroorganismen und Zellkulturen GmbH (DSM). Starch, maltooligosaccharide and/or the like may be further added as a sugar source.

Purification of the Novel Transferase of the Present Invention

The novel transferase of the present invention which is produced by the above-described microorganisms can be extracted as follows: At first, the bacterial bodies are collected from the culture obtained in a culturing process as described above by a publicly-known procedure, for example, by centrifugation; the resultant is suspended in a proper buffer solution; the bacterial bodies are then crushed by freeze thawing, a ultrasonic treatment, grinding and/or the like; and the resultant is centrifuged or filtrated to obtain a cell extract containing the objective transferase.

To purify the novel transferase of the present invention which is contained in the cell extract, publicly-known processes for isolation and purification can be employed in proper combination. Examples of such processes may include a process utilizing solubility, such as salt precipitation and solvent precipitation; a process utilizing difference in molecular weight, such as dialysis, ultrafiltration, gel filtration and SDS-Polyacryl-amide gel electrophoresis; a process utilizing a difference in electric charge, such as ion exchange chromatography; a process utilizing specific affinity, such as affinity chromatography; a process utilizing a difference in hydrophobicity, such as hydrophobic chromatography and reversed phase chromatography; and further, a process utilizing a difference in isoelectric point, such as isoelectric focusing. Practical examples of these processes are shown in Examples I-2-I-5 below. Finally, Native Polyacrylamide gel electrophoresis, SDS-Polyacrylamide gel electrophoresis or isoelectric focusing is performed to obtain a purified enzyme which appears therein as a single band.

As to measurement of activity in the enzyme or enzyme-containing substance isolated by the above various purification processes, starch is used as the substrate in the activity-measuring method offered by Lama, et al. By this method, though the production of trehalose and glucose can be confirmed, the production of trehaloseoligosaccharides cannot be detected at all, and as a serious problem, even the trehalose-producing activity becomes undetectable due to its disappearance during purification. Therefore, the purification and characterization of the true substance of the enzyme activity had been substantially impossible. Under such circumstances, Inventors employed a new activity-measuring method in which the substrate is a maltooligosaccharide such as maltotriose, and the index is activity of producing a trehaloseoligosaccharide such as glucosyltrehalose. As a result, isolation and purification of the objective enzyme could be achieved for the first time by-this method, and finally, the true substance of the novel transferase activity of the present invention could be practically purified and specified.

Characteristics of the Novel Transferase according to the Present Invention

As examples of the enzyme of the present invention, the transferases produced by the Sulfolobus solfataricus strain KM1, the Sulfolobus solfataricus strain DSM 5833, the Sulfolobus acidocaldarius strain ATCC 33909, and the Acidianus brierleyi strain DSM 1651, respectively, are taken up, and the enzymatic characteristics of these transferases are shown in Table 1 below in summary. Here, data in the table is based on the practical examples shown in Examples I-6 and 1-7.

TABLE 1

| | | Sulfolobus | Sulfolobus | Sulfolobus | Acidianus |
|---|---|---|---|---|---|
| | | solfataricus | solfataricus | acidocaldarius | brierleyi |
| Physicochemical properties | | KM1 | DSM5833 | ATCC33909 | DSM1651 |
| (1) | Enzyme action and | Acts on glucose polymers composed of more than maltotriose | | | |
| | Substrate specificity | wherein glucoses are α-1, 4-linked, so as to combine two sugar | | | |
| | | moieties from the reducing end into an α-1, α-1 linkage by | | | |
| | | transfer. Not acts on maltose or glucose. | | | |
| (2) | Optimum pH | 5.0-6.0 | 4.5-5.5 | 4.5-5.5 | 4.5-5.5 |
| (3) | pH Stability | 4.0-10.0 | 4.5-12.0 | 4.0-10.0 | 4.0-12.0 |
| (4) | Optimum temperature | 60-80° C. | 70-80° C. | 70-80° C. | 70-80° C. |
| (5) | Thermal stability | 85° C., 6 hr | 85° C., 6 hr | 85° C., 6 hr | 85° C., 6 hr |
| | | 91% remained | 90% remained | 90% remained | 98% remained |
| (6) | Molecular weight | | | | |
| | SDS-PAGE | 76000 | 75000 | 74000 | 74000 |
| | Gel-permeation | 54000 | 56000 | 56000 | 135000 |
| (7) | Isoelectric point | 6.1 | 5.3 | 5.6 | 6.3 |
| (8) | Inhibitor | 5 mM CuSO₄ | 5 mM CuSO₄ | 5 mM CuSO₄ | 5 mM CuSO₄ |
| | | 100% inhibited | 100% inhibited | 100% inhibited | 100% inhibited |

Note 1: Time-course Change

When maltotriose was used as the substrate, glucosyltrehalose as a product in the principal reaction, and besides, equal moles of maltose and glucose were produced as products in a side reaction.

When a saccharide having a polymerization degree, n, which is equal to or higher than that of maltotetraose, was used, a saccharide of which the glucose residue at the reducing end is α-1,α-1-linked was produced in the principal reaction, and besides, equal moles of glucose and a saccharide having a polymerization degree of n−1 were produced in a side reaction.

Note 2: Enzymatic Action/Mode of Enzymatic Reaction

It is considered that the enzyme has an activity of acting on maltotriose or a larger saccharide, three glucose residues from the reducing end of the sacdharide being α-1,4-linked, so as to transfer the first linkage from the reducing end into an α-1,α-1-linkage. As a side reaction, the enzyme also has an activity of liberating glucose from a glucose polymer, when, for example, the concentration of the substrate is low, or the reaction time is long. The details are as shown in the practical example of Example I-7.

The characteristics of the present enzyme have been described above. As described in the above item titled “Enzymatic Action/Mode of Enzymatic Reaction”, the present enzyme has an activity of acting on maltotriose or a larger saccharide, three glucose residues from the reducing end of the saccharide being α-1,4-linked, so as to transfer the first linkage from the reducing end into an α-1,α-1-linkage, and such an activity is quite a novel enzymatic activity. However, as obvious in the examples below, the characteristics of the present enzyme other than such enzymatic activities slightly vary according to the difference in genus or species between the bacterial strains.

Production of Trehaloseoligosaccharides such as Glucosyltrehalose and Maltooligosyltrehalose

The present invention provides a process for producing a saccharide having an end composed of a couple of α-1,α-1-linked sugar units, characterized in that the enzyme of the present invention is used and allowed to act on a substrate saccharide, the substrate saccharide being composed of at least three sugar units wherein at least three glucose residues from the reducing end are α-1,4-linked, so as to produce the objective saccharide in which at least three sugar units from the reducing end side are glucose residues and the linkage between the first and second glucose residues from the reducing end side is α-1,α-1 while the linkage between the second and third glucose residues from the reducing end side is α-1,4. The process according to the present invention will be illustrated below with the most typical example, namely, with a process for producing trehaloseoligosaccharides such as glucosyltrehalose and maltooligosyltrehaloses.

In the process for producing trehaloseoligosaccharides such as glucosyltrehalose and maltooligosyltrehaloses according to the present invention, trehaloseoligosaccharides such as glucosyltrehalose and maltooligosyltrehaloses are produced from a saccharide such as maltooligosaccharides, typically, from each or a mixture of maltooligosaccharides by the present enzyme derived from archaebacteria. Accordingly, the mode of contact between the present transferase and a saccharide such as maltooligosaccharides is not specifically limited as long as the present enzyme produced by archaebacteria can act on the saccharide such as maltooligosaccharides in such mode. In practice, the following procedure may ordinarily be performed: A crude enzyme is obtained from the bacterial bodies or crushed bacterial bodies of an archaebacterium; and the purified enzyme obtained in each of the various purification steps, or the enzyme isolated and purified through various purification means, is made to act directly on a saccharide such as maltooligosaccharides. Alternatively, the above-described enzyme may be put into contact with a saccharide such as maltooligosaccharides in a form of a immobilized enzyme which is immobilized to a carrier in the usual way. Additionally, two or more of the present enzymes derived from two or more species of archaebacteria may coexist and be put into contact with a saccharide such as maltooligosaccharides.

The mixture of maltooligosaccharides, which is a typical raw material of the substrate in the above-described producing process of the present invention, may be prepared, for example, by properly hydrolyzing. or acidolyzing starch using an endotype amylase, a debranching enzyme or the like so that at least three glucose residues from the reducing end of the product are α-1,4-linked. The endotype amylases to be used herein may include enzymes derived from bacteria belonging to the genus Bacillus, fungi belonging to the genus Aspergillus, and plants such as malt, and others. On the other hand, the debranching enzymes to be used herein may include pullulanase derived from bacteria belonging to the genus Bacillus, Klebsiella or the like, or isoamylase derived from bacteria belonging to the genus Pseudomonas. Further, these enzymes may be used in combination.

The concentration of a saccharide such as maltooligosaccharides should be suitably selected within the range in which the saccharide to be used is dissolved, considering the specific activity of the present enzyme, the reaction temperature and others. A range of 0.5-70% is ordinary, and a range of 5-40% is preferable. The reaction temperature and pH condition in the reaction of the saccharide with the enzyme should be optimum for the present transferase. Accordingly, the reaction is performed ordinarily at 50-85° C. and pH 3.5-6.5, approximately, and more preferably, at 60-80C and pH 4.5-6.0.

The produced reaction mixture which contains trehaloseoligosaccharides such as glucosyltrehalose or maltooligosyltrehalose can be purified according to a publicly-known process. For example, the obtained reaction mixture is desalted with an ion-exchange resin; the objective saccharide fraction is then isolated and crystallized by chromatography using activated charcoal, an ion-exchange resin (HS03 type), cation-exchange resin (Ca type) or the like as a separating material, and by a subsequent condensation to be optionally performed; and finally, trehaloseoligosaccharides are yielded within a high purity.

A Gene Coding for the Novel Transferase

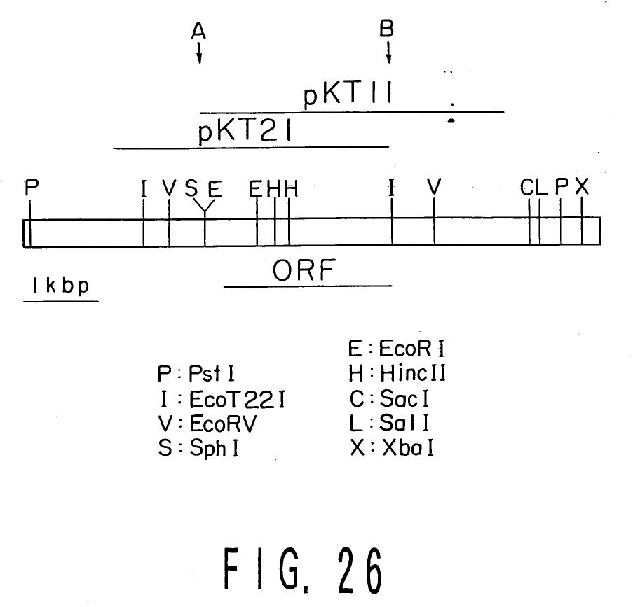

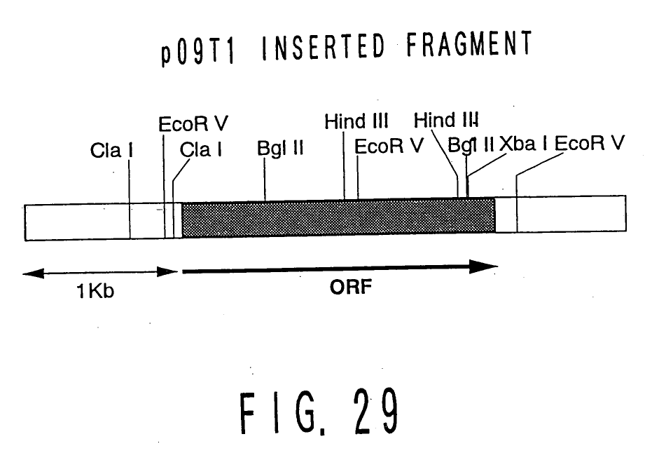

According to the present invention, a gene coding for the above novel transferase is further provided. For example, the DNA fragments illustrated by restriction maps shown in FIGS. 26 and 29 can be listed as DNA fragments comprising a gene coding for the novel transferase according to the present invention.

These DNA fragment can be obtain from an archaebacterium belonging to the order Sulfolobales, and preferably, belonging to the genus Sulfolobus. More preferably, the fragment can be isolated from the below-described Sulfolobus solfataricus strain KM1 or Sulfolobus acidocaldarius strain ATCC 33909. The suitable process for the isolation from the Sulfolobus solfataricus strain KM1 or the Sulfolobus acidocaldarius strain ATCC 33909 is illustrated in detail in the below-described Examples.

The practical examples of the origin from which the DNA fragments can be derived may further include the Sulfolobus solfataricus strains DSM 5354, DSM 5833, ATCC 35091 and ATCC 35092; the Sulfolobus acidocaldarius strain ATCC 49426; the Sulfolobus shibatae strain DSM 5389; the Acidianus brierleyi strain DSM 1651; and others. It is obvious from the following facts that these archaebacteria can be the origins of the DNA fragments according to the present invention: The novel transferase gene derived from the Sulfolobus solfataricus strain KM1 forms a hybrid with the chromosome DNA derived from each of those archaebacteria in the below-described hybridization test performed in Example 1-17; and further, the characteristics of the enzymes themselves very closely resemble each other as described above. Moreover, the results in the aforementioned Example suggestively indicate that the novel transferase gene according to the present invention is highly conserved, specifically in archaebacteria belonging to the order Sulfolobales.

The preferable mode for carrying out the present invention provides a DNA fragment comprising a DNA sequence. coding for the amino acid sequence shown in Sequence No. 2 or 4 as a suitable example of the gene coding for the novel transferase of the present invention. Further, the sequence from 335th base to 2518th base among the base sequence shown in Sequence No. 1 can be listed as a suitable example of the DNA sequence coding for the amino acid sequence shown in Sequence No. 2. The sequence from 816th base to 2855th base among the base sequence shown in Sequence No. 3 can be listed as a suitable example of the DNA sequence coding for the amino acid sequence shown in Sequence No. 4.

In general, when given the amino acid sequence of a protein, the base sequence coding therefor can be easily determined by referring to what is called the Codon Table. Therefore, several base sequences which code for the amino acid sequence shown in Sequence No. 2 or 4 can be suitably selected. Accordingly, in the present invention, “the DNA sequence coding for the amino acid shown in Sequence No. 2” implies the DNA sequence comprising the sequence from 335th base to 2518th base of the base sequence shown in Sequence No. 1; and also, the DNA sequences which comprise the same base sequence as above except that one or more codons are replaced with the codons having a relationship of degeneracy therewith, and which still code for the amino acid shown in Sequence No. 2. Similarly, “the DNA sequence coding for the amino acid shown in Sequence No. 4” implies the DNA sequence comprising the sequence from 816th base to 2855th base of the base sequence shown in Sequence No. 3; and also, the DNA sequences which comprise the same base sequence as above except that one or more codons are replaced with the codons having a relationship of degeneracy therewith, and which still code for the amino acid shown in Sequence No. 4.

Further, as described below, the scope of the novel transferase according to the present invention also includes the sequences equivalent to the amino acid sequence shown in Sequence No. 2 or 4. The scope of the DNA fragment according to the present invention, therefore, further includes the base sequences which code for such equivalent sequences.

Incidentally, Inventors surveyed the existence of a base sequence homologous to the base sequence shown in Sequence No. 1 or 3 through a data bank on base sequences (EMBL) by using sequence-analyzing software, GENETYX (by Software Development Co.). As a result, Inventors have confirmed that such a base sequence does not exist.

Since the base sequence of the DNA fragment comprising the sequence from 335th base to 2518th base of the base sequence shown in Sequence No. 1, and the base sequence of the DNA fragment comprising the sequence from 816th base to 2518th base of the base sequence shown in Sequence No. 3 have been determined, a means for obtaining these DNA fragments is producing them based on a process for polynucleotide synthesis.

Further, these sequences can be obtained by using a process of gene engineering from the above-described archaebacteria belonging to the order Sulfolobales, and preferably, from the Sulfolobus solfataricus strain KM1 or the Sulfolobus acidocaldarius strain ATCC 33909. For example, they can be suitably obtained by a process described in Molecular Cloning: A Laboratory Manual [Sambrook, Mainiatis, et al., published by Cold Spring Harbour Laboratory Press (1989)], and others. The practical method is illustrated in detail in the below-described examples.

Recombinant Novel Transferase

Since the gene coding for the novel transferase is provided as described above, the expressed product from this gene, a recombinant novel transferase, can be obtained according to the present invention.

Suitable examples of the recombinant novel transferase according to the present invention may include an expressed product from the DNA fragment illustrated with the restriction map shown in FIG. 26 or 29.

Also, the suitable examples may include a polypeptide comprising the amino acid sequence shown in Sequence No. 2 or 4 of the Sequence Table, or the equivalent sequence thereof. Here, the term “equivalent sequence” stands for the amino acid sequence which basically has the amino acid sequence shown in Sequence No. 2 or 4; but has undergone insertion, replacement or deletion of some amino acids, or addition of some amino acids to each terminus; and still keeps the activity of the novel transferase. The state in which the equivalent sequence keeps the activity of the novel transferase means that it keeps an activity sufficient for similar use in similar conditions as compared to the polypeptide having the complete sequence shown in Sequence No. 2 or 4, when the activity is applied in a practical mode for use. Obviously, persons skilled in the art can select and produce such an “equivalent sequence” by referring to the sequences shown in Sequence Nos. 2 and 4 without any special difficulty, since it is revealed in Example I-18 that the same activity is kept in the enzymes derived from the Sulfolobus solfataricus strain KM1 and the Sulfolobus acidocaldarius strain ATCC 33909 though the homology between the amino acid sequences of the novel transferases from these 2 strains is 49% when calculated considering gaps.

As clarified in Example I-17 below, each. of the DNA fragments having the sequences shown in Sequence Nos. 1 and 3, respectively, can hybridize with each of DNA fragments derived from some bacterial strains other than the Sulfolobus solfataricus strain KM1 and the Sulfolobus acidocaldarius strain ATCC 33909 which are the origins of said DNA fragments, respectively. Meanwhile, as described above, Inventors have now confirmed the existence of a novel transferase having very close characteristics in those bacterial strains. Further, as revealed in Example I-18 below, the homology between the amino acid sequences of the novel transferases derived from the Sulfolobus solfataricus strain KM1 and the Sulfolobus acidocaldarius strain ATCC 33909 is 49% when calculated considering gaps. It is, therefore, obvious to persons skilled in the art that the activity of the novel transferase can be kept in a sequence which is homologous, to some extent, with the amino acid sequence shown in Sequence No. 2 or 4.

Incidentally, Inventors surveyed the existence of a sequence homologous to the amino acid sequence shown in Sequence No. 2 or 4 through a data bank on amino acid sequences (Swiss prot and NBRF-PFB) by using sequence-analyzing software, GENETYX (by Software Development Co.). As a result, Inventors have confirmed that such a sequence does not exist.

Expression of a Gene Coding for the Novel Transferase

The recombinant novel transferase according to the present invention can be produced in a host cell by transforming the host cell with a DNA molecule, and especially with an expression vector, which can replicate in the host cell, and contains the DNA fragment coding for the novel transferase according to the present invention so as to express the transferase gene.

The present invention, therefore, further provides a DNA molecule, and particularly, an expression vector, which contains a gene coding for the novel transferase according to the present invention. Such a DNA molecule can be obtained by integrating the DNA fragment coding for the novel transferase of the present invention into a vector molecule. According to the preferable mode for carrying out the present invention, the vector is a plasmid.

The DNA molecule according to the present invention can be prepared on the basis of the process described in the aforementioned Molecular Cloning: A Laboratory Manual.

The vector to be used in the present invention can suitably be selected from viruses, plasmids, cosmid vectors, and others considering the type of the host cell to be used. For example, a bacteriophage of A phage type, a plasmid of pBR or pUC type can be used when the host cell is Escherichia coli; a plasmid of pUB type can be used when the host cell is Bacillus subtilis; and a vector of YEp or YCp type can be used when the host cell is yeast.

The plasmid should preferably contain a selective marker for detection of the transformant, and a drug-resistance marker and an auxotrophy marker can be used as such a selective marker.

Further, the DNA molecule as an expression vector according to the present invention should preferably contain DNA sequences necessary for expression of the novel transferase gene, for example, a transcription-controlling signal, a translation-controlling signal and/or the like such as a promoter, a transcription-initiating signal, a ribosome-binding site, a translation-stopping signal, and a transcription-finishing signal.

Examples of the promoter to be suitably used may include, as well as a promoter functional in the host which contains the insertional fragment, a promoter such as a lactose operon (lac) and a tryptophan operon (trp) for Escherichia coli, a promoter such as an alcohol dehydrogenase gene (ADH), an acid phosphatase gene (PHO), a galactose gene (GAL), and a glyceraldehyde 3-phosphate dehydrogenase gene (GPD) for yeast.

Here, the base sequence comprising the sequence from 1st base to 2578th base of the base sequence shown in Sequence No. 1, and the base sequence comprising the sequence from 1st base to 3467th base of the base sequence shown in Sequence No. 3 are recognized as containing the aforementioned sequences necessary for expression. It is, therefore, also suitable to use these sequences as they are.

Moreover, when the host cell is Bacillus subtilis or yeast, it will be advantageous to use a secretory vector so as to excrete the recombinant novel transferase outside of the host's body.

In addition to Escherichia coli, Bacillus subtilis, yeast, and advanced eukaryotes, can be used as a host cell. Microorganisms belonging to the genus Bacillus such as Bacillus subtilis are suitably used. Some strains belonging to this genus are known to excrete a protein outside of the bacterial body in a large amount. Therefore, a large amount of the recombinant novel amylase can be excreted in the culture medium by using a secretory vector. This is preferable because the purification from the supernant of the culture will be easy. Further, some strains belonging to the genus Bacillus are known to excrete a very little amount of protease outside of the bacterial body. It is preferable to use such strains because the recombinant novel amylase can be efficiently produced thereby. Moreover, it will be very advantageous to select a microorganism which does not produce glucoamylase and to use it as a host cell, because the recombinant novel transferase of the present invention which is obtained as a cell extract or a simply-purified crude enzyme can be directly used for the below-described production of trehaloseoligosaccharides.

The recombinant novel transferase produced by the aforementioned transformant can be obtained as follows: At first, the above-described host cell is cultivated under proper conditions; the bacterial bodies are. collected from the resultant culture by a publicly-known method, for example, by centrifugation, and suspended in a proper buffer solution; the bacterial bodies are then crushed by freeze thawing, a ultrasonic treatment, grinding and/or the like; and the resultant is centrifuged or filtrated to obtain a cell extract containing the recombinant novel transferase.

Purification of the recombinant novel transferase existing in the cell extract can be performed by a proper combination of publicly-known processes for isolation and purification. Examples of the processes may include a process utilizing a difference in thermostability, such as a heat treatment; a process utilizing a difference in solubility, such as salt precipitation and solvent precipitation, a process utilizing a difference in molecular weight, such as dialysis, ultrafiltration, gel filtration and SDS-Polyacryl-amide gel electrophoresis; a process utilizing a difference in electric charge, such as ion exchange chromatography; a process utilizing specific affinity, such as affinity chromatography; a process utilizing a difference in hydrophobicity, such as hydrophobic chromatography and reversed phase chromatography; and further, a process utilizing a difference in isoelectric point, such as isoelectric focusing. Since the recombinant novel transferase is thermostable, the purification can be very easily performed using heat treatment, by which proteins in the host can be denatured and made into precipitation suitable for removal.

Production of Trehaloseoligosaccharides Using the Recombinant Novel Transferase

The present invention further provides a process for producing so called trehaloseoligosaccharide such as glucosyltrehalose and maltooligosyltrehalose, wherein the above-described recombinant novel transferase is used.

Specifically, the process according to the present invention is a process for producing a trehaloseoligosaccharide in which at least three sugar units from the reducing end side are glucose residues and the linkage between the first and second glucose residues from the reducing end side is α-1,α-1 while the linkage between the second and third glucose residues from the reducing end side is α-1,4. And the process comprises putting the above-described recombinant novel transferase into contact with a saccharide, the saccharide being composed of at least three sugar units wherein at least three glucose residues from the reducing end are α-1,4-linked.

Though the saccharide composed of at least three sugar units in which at least three glucose residues from the reducing end are α-1,4-linked is not specifically limited, starch, starch hydrolysate, maltooligosaccharides, and others can be listed as an example of such a saccharide. Examples of starch hydrolysate may include a product produced by properly hydrolyzing or acidolyzing starch using an endotype amylase, a debranching enzyme or the like so that at least three glucose residues from the reducing end of the product are α-1,4-linked. Examples of endotype amylase to be used herein may include enzymes derived from bacteria belonging to the genus Bacillus, fungi belonging to the genus Aspergillus, and plants such as malt, and others. On the other hand, Examples of the debranching enzymes may include pullulanase derived from bacteria belonging to the genus Bacillus, Klebsiella or the like, or isoamylase derived from bacteria belonging to the genus Pseudomonas. Further, these enzymes may be used in combination.

The mode and conditions for contact between the recombinant novel transferase of the present invention and the saccharide composed of at least three sugar units in which at least three glucose residues from the reducing end are α-1,4-linked is not specifically limited as long as the recombinant novel transferase can act on the saccharide therein. An example of a suitable mode for performing the contact in a solution is as follows. The concentration of a saccharide such as maltooligosaccha-rides should be suitably selected within the range in which the saccharide to be used is dissolved, considering the specific activity of the recombinant novel transferase, the reaction temperature and others. A range of 0.5-70% is ordinary, and a range of 5-40% is preferable. The reaction temperature and pH condition in the reaction of the saccharide with the enzyme should be optimum for the recombinant novel transferase. Accordingly, the reaction is performed ordinarily at 50-85° C. and pH 3.5-6.5, approximately, and more preferably, at 60-80° C. and pH 4.5-6.0.

Additionally, the purification degree of the recombinant novel transferase can be properly selected. For example, a crude enzyme derived from the crushed bodies of a transformant can be used as it is, and the purified enzyme obtained in each of the various purification steps can be also used, and further, the enzyme isolated and purified through various purification means can be used.

Alternatively, the above-described enzyme may be put into contact with a saccharide such as maltooligosaccharides in a form of a immobilized enzyme which is immobilized to a carrier in the usual way.

The produced trehaloseoligosaccharides such as glucosyltrehalose and maltooligosyltrehalose can be recovered by purifying the reaction mixture using according to a publicly-known process. For example, the obtained reaction mixture is desalted with an ion-exchange resin; the objective saccharide fraction is then isolated and crystallized by chromatography using activated charcoal, an ion-exchange resin (HS03 type), cation-exchange resin (Ca type) or the like as a separating material, and by a subsequent condensation to be optionally performed; and finally, trehaloseoligosaccha-rides are yielded within a high purity.

II. Novel Amylase

Microorganisms Producing Novel Amylase of the Present Invention

Examples of the archaebacteria to be used in the present invention may include the Sulfolobus solfataricus strain KM1 (the above-described novel bacterial strain which was substantially purely isolated from nature by Inventors), the Sulfolobus solfataricus strain DSM 5833, and the Sulfolobus acidocaldarius strain ATCC 33909 (DSM 639).

As described above, a fairly wide variety of archaebacteria taxonomically classified under the order Sulfolobales may be considered as the microorganisms which can produce the novel amylase of the present invention. Here, the archaebacterium belonging to the order Sulfolobales are taxonomically defined as being highly acidophilic (capable of growing in a temperature range of 55-88° C.), being thermophilic (capable of growing in a pH range of 1-6), being aerobic, and being sulfur bacteria (being coccal bacteria having no regular form and a diameter of 0.6-2 μm). The aforementioned Sulfolobus solfataricus strain DSM 5833 had formerly been named as Caldariella acidophila. From the fact like this, microorganisms which are closely related to the above-described archaebacteria genetically or taxonomically and which are capable of producing the enzyme of the same kind, and mutants derived from these strains by treatment with various mutagens can be used in the present invention. Among the above-illustrated microorganisms, the Sulfolobus solfataricus strain KM1 is the bacterial strain which Inventors isolated from a hot spring in Gunma Prefecture, and the characteristics and deposition of this strain are as described above in detail.

Cultivation of the Microorganisms which Produce the Novel Amylase of the Present Invention

The culture conditions for producing the novel amylase of the present invention may suitably be selected within ranges in which the objective amylase can be produced. When a concussion culturing or a culturing with aeration and stirring using a liquid medium is employed, the culturing for 2-7 days should suitably be performed at a pH and a temperature which allow the growth of each microorganism. The culture medium to be suitably used is, for example, any of the culture media which are described in Catalogue of Bacteria and Pharges 18th edition (1992). published by American Type Culture Collection (ATCC), and in Catalogue of Strains 5th edition (1993) published by Deutsche Sammlung von Mikroorganismen und Zellkulturen GmbH (DSM). Starch, maltooligosaccharide and/or the like may be further added as a sugar source.

Purification of the Novel Amylase of the Present Invention

The novel amylase of the present invention which is produced by the above-described microorganisms can be extracted as follows: At first, the bacterial bodies.are collected from the culture obtained in a. culture process as described above by a publicly-known procedure, for example, by centrifugation; the resultant is suspended in a proper buffer solution; the bacterial bodies are then crushed by freeze thawing, an ultrasonic treatment, grinding and/or the like; and the resultant is centrifuged or filtrated to obtain a cell extract containing the objective amylase.

To purify the novel amylase of the present invention which is contained in the cell extract, publicly-known processes for isolation and purification can be employed in a proper combination. Examples of such processes may include a process utilizing solubility, such as salt precipitation and solvent precipitation; a process utilizing a difference in molecular weight, such as dialysis, ultrafiltration, gel filtration and SDS-Polyacryl-amide gel electrophoresis; a process utilizing a difference in electric charge, such as ion exchange chromatography; a process utilizing specific affinity, such as affinity chromatography; a process utilizing a difference in hydrophobicity, such as hydrophobic chromatography and reversed phase chromatography; and further, a process utilizing a difference in isoelectric point, such as isoelectric focusing. The practical examples of these processes are shown in Examples II-2-II-4 below. Finally, Native Polyacrylamide gel electrophoresis, SDS-Polyacrylamide gel electrophoresis or isoelectric focusing is performed to obtain a purified enzyme which appears therein as a single band.

As to measurement of activity in the enzyme or enzyme-containing substance isolated by the above various purification processes, starch is used as the substrate in the activity-measuring method offered by Lama, et al. By this method, when various amylases coexist in the reaction system, the production of starch hydrolysate can be detected. In contrast, when each of the individually isolated products of these amylases is used, both of the detecting sensitivity and quantifying ability become low, and as a serious problem, the starch-hydrolyzing activity becomes undetectable due to its disappearance during purification. Therefore, the purification and characterization of the true substance of the enzyme activity had been substantially impossible. Under such circumstances, Inventors employed a new activity-measuring method in which the substrate is a trehaloseoligosaccharide such as maltotriosyltrehalose, and the index is activity of hydrolyzing it into α,α-trehalose and maltooligosaccharides such as maltotriose. As a result, this method was found to have an extremely high specificity, detecting sensitivity and quantifying ability, and isolation and purification of the objective enzyme could be achieved for the first time, and finally, the true substance of the novel amylase activity of the present invention could be practically purified and specified.

Characteristics of the Novel Amylase According to the Present Invention

As examples of the enzyme of the present invention, the amylases produced. by the Sulfolobus solfataricus strain KM1, the Sulfolobus solfataricus strain DSM 5833, and the Sulfolobus acidocaldarius strain ATCC 33909 (DSM 639), respectively, are taken up, and the enzymatic characteristics of these amylases are shown in Table 2 below in summary. Here, the data in the table are based on the practical examples shown in Example II-5.

TABLE 2

| | | Sulfolobus | Sulfolobus | Sulfolobus |
|---|---|---|---|---|
| | | solfataricus | solfataricus | acidocaldarius |
| Physicochemical properties | | KM1 | DSM5833 | ATCC33909 |
| (1) | Enzyme action and | Acts on glucose polymers composed of more than maltotriose, | | |
| | Substrate specificity | so as to hydrolyze by endo-type and liberates principally | | |
| | | monosaccharide or disaccharide from the reducing end. | | |
| | | Especially liberates α,α-trehalose from trehaloseoligo- | | |
| | | saccharide wherein the linkage between two glucoses from the | | |
| | | reducing end side is α-1, α-1 while the other linkages are α-1, 4. | | |
| (2) | Optimum pH | 4.5-5.5 | 4.5-5.5 | 5.0-5.5 |
| (3) | pH Stability | 3.5-10.0 | 3.0-12.0 | 4.0-13.0 |
| (4) | Optimum temperature | 70-85° C. | 70-85° C. | 60-80° C. |
| (5) | Thermal stability | 85° C., 6 hr | 85° C., 6 hr | 80° C., 6 hr |
| | | 100% remained | 100% remained | 100% remained |
| (6) | Molecular weight | 61000 | 62000 | 64000 |
| | SDS-PAGE | | | |
| (7) | Isoelectric point | 4.8 | 4.3 | 5.4 |
| (8) | Inhibitor | 5 mM CuSO₄ | 5 mM CuSO₄ | 5 mM CuSO₄ |
| | | 100% inhibited | 100% inhibited | 100% inhibited |

Note 1: Time-course Change

When soluble starch was used as the substrate, the iodine-starch complex quickly disappeared in the early stage of the enzymatic reaction, and subsequently, the hydrolyzing reaction progressed so as to produce maltose and glucose as principal products, and maltotriose and maltotetraose in slight amounts.

Note 2: Enzymatic Action/Mode of Enzymatic Reaction

The present enzyme principally produces glucose and maltose, and produces small amounts of maltotriose and maltotetraose, when starch, starch hydrolysate and/or maltooligosaccharide are used as the substrate. As to the action mechanisms, the present enzyme has an amylase activity of endotype-hydrolyzing these substrates, and an activity of producing principally monosaccharide and/or disaccharide from the reducing end side.

In particular, the enzyme has a high reactivity to a saccharide composed of at least three sugar units wherein the linkage between the first and the second glucose residues from the reducing end side is α-1,α-1 while the linkage between the second and third glucose residues from the reducing end side is α-1,4 (for example, trehaloseoligosaccharide). When these saccharides are used as the substrate, the enzyme has an activity of hydrolyzing the α-1,4 linkage between the second and third glucose residues from the reducing end side, and specifically liberates α,α-trehalose in the early stage of the reaction.

Consequently, the present enzyme can be recognized as a novel amylase. The details are as practically described in Example II-5.

The characteristics of the present enzyme have been described above. However, as is obvious from Table 2 and the examples below, the characteristics of the present enzyme other than such enzymatic activities are found to slightly vary according to the difference in genus or species between the bacterial strains.

Transferase to be Used in Production of α,α-Trehalose

The transferase of the present invention which is described in detail in the above-described item “I. Novel Transferase” can be used for production of α,α-trehalose according to the present invention. Specifically, examples of such a transferase may include transferases derived from the Sulfolobus solfataricus strain ATCC 35091 (DSM 1616), the Sulfolobus solfataricus strain DSM 5833, the Sulfolobus solfataricus strain KM1, the Sulfolobus acidocaldarius strain ATCC 33909 (DSM 639), and the Acidianus brierleyi strain DSM 1651.

These transferases can be produced according to, for example, the processes described in Examples I-2-I-5 below. The transferases thus obtained have various characteristics shown in Example I-6 below.

Production of α,α-Trehalose

The present invention provides a process for producing α,α-trehalose by using the novel amylase and transferase of the present invention. The process according to the present invention will be illustrated below with the most typical example, namely, with a process for producing α,α-trehalose from a glucide raw material such as starch, starch hydrolysate and/or maltooligosaccharide. Incidentally, the probable reaction-mechanisms of the above two enzymes are considered as follows: At first, the novel amylase of the present invention acts on starch, starch hydrolysate or maltooligosaccharide by its endotype-hydrolyzing activity to produce amylose or maltooligosaccharide; subsequently, the first α-1, 4 linkage from the reducing end of the resultant amylose or maltooligosaccharide is transferred into an α-1,α-1 linkage by the activity of the transferase; further, the novel amylase acts again to produce α,α-trehalose, and amylose or maltooligosaccharide which is deprived of the polymerization degree by two; and the amylase or maltooligosaccharide thus derived undergoes the above reactions repeatedly, so that α,α-trehalose would be produced in a high yield.

Such reaction mechanisms may be attributed to the specific reaction-mode as follows, which is possessed by the novel amylase of the present invention: The enzyme has a higher reactivity to a saccharide composed of at least three sugar units wherein the linkage between the first and the second glucose residues from the reducing end side is α-1,α-1 while the linkage between the second and third glucose residues from the reducing end side is an α-1,4 (for example, trehaloseoligosac-charide), as compared with the reactivity to each of the corresponding maltooligosaccharide; and the enzyme specifically hydrolyzes the α-1,4 linkage between the second and third glucose residues from the reducing end side of the above saccharide, and liberates α,α-trehalose.

As far as Inventors know, there is no formerly-known amylase which can act on maltooligosyltrehalose derived from maltooligosaccharide by modifying the reducing end with an α-1,α-1 linkage, and-which has an activity of specifically hydrolyzing the α-1,4 linkage next to the α-1,α-1 linkage to liberate α,α-trehalose in a high yield. Accordingly, it has been almost impossible to produce α,α-trehalose in a high yield.

In the process for producing α,α-trehalose according to the present invention, the mode of contact between the present amylase and transferase, and starch, starch hydrolysate and/or maltooligosaccharides is not specifically limited as long as the amylase of the present invention (the present enzyme) produced by archaebacteria can act on the starch, starch hydrolysate and/or maltooligosaccharides in such mode. In practice, the following procedure may ordinarily be performed: A crude enzyme is obtained from the bacterial bodies or crushed bacterial bodies of an archaebacterium; and the purified enzyme obtained in each of the various purification steps, or the enzyme isolated and purified through various purification means, is made to act directly on glucide such as starch, starch hydrolysate and maltooligosaccharide. Alternatively, the enzyme thus obtained may be put into contact with glucide such as starch, starch hydrolysate and maltooligosaccharide in a form of a immobilized enzyme which is immobilized to a carrier. Additionally, two or more of the present enzymes derived from two or more species of archaebacteria may coexist and be put into contact with glucide such as starch, starch hydrolysate and maltooligosaccharide.

In the process for producing α,α-trehalose according to the present invention, the above-described amylase and transferase should be used in amounts within the optimum ranges. An excess amount of amylase will act on the starch, starch hydrolysate or maltooligosaccharide on which the transferase have not acted to modify its reducing end, while an excess amount of transferase will, in the side reaction, hydrolyze the trehaloseoligo-saccharide such as maltooligosyltrehalose which has been produced by the transferase itself, and produce glucose.

The practical concentrations of the amylase and transferase relative to the amount of substrate are 1.5 U/ml or higher, and 0.1 U/ml or higher, respectively. Preferably, the concentrations should be 1.5 U/ml or higher, and 1.0 U/ml or higher, respectively, and more preferably, 15 U/ml or higher, and 1.0 U/ml or higher, respectively. Meanwhile, the ratio of amylase concentration to transferase concentration should be 100-0.075, and preferably, 40-3.

The concentration of glucide such as starch, starch hydrolysate and maltooligosaccharide should be suitably selected within the range in which the glucide to be used is dissolved, considering the specific activity of each enzyme to be used, the reaction temperature, and others. A range of 0.5-70% is ordinary, and a range of 5-40% is preferable. The reaction temperature and pH condition in the reaction of the glucide with the enzymes should be optimum for the amylase and the transferase. Accordingly, the reaction is performed ordinarily at 50-85° C. and pH 3.5-8, approximately, and more preferably, at 60-75° C. and pH 4.5-6.0.

Additionally, when the glucide raw material to be used is starch, starch hydrolysate or the like having a high polymerization degree, the production of α,α-trehalose can be further promoted by using another endotype liquefying amylase together as a supplement. Such a debranching enzyme as pullulanase and isoamylase can also be used herein. The endotype amylase, pullulanase, isoamylase or the like may not be such an enzyme as derived from archaebacteria, and therefore, it is not specifically limited. For example, amylase derived from bacteria belonging to the genus Bacillus, fungi belonging to the genus Aspergillus and plants such as malt, and others can be used. The debranching enzyme may be pullulanase (including thermostable pullulanase) derived from bacteria belonging to the genus Bacillus, Klebsiella or the like, or isoamylase derived from bacteria belonging to the genus Pseudomonas. Further, these enzymes may be used in combination.

However, the addition of an excess amount of amylase will possibly cause production of glucose and maltose which the transferase will not act on. Similarly, the addition of an excess amount of a debranching enzyme will cause a decrease in solubility of the substrate due to cleavage of the 1,6-linkage, and lead to production of a highly-viscous and insoluble substance (amylose). For that reason, the amounts of amylase and the debranching enzyme should carefully be controlled so as not to produce excessive glucose, maltose, or an insoluble substance. As to debranching enzymes, the concentration should be properly selected within a range in which an insoluble substance is not produced, considering the specific activity of the present amylase, the reaction temperature, and the like. Specifically, when the treatment is performed at 40° C. for one hour, the ordinary concentration relative to the substrate is within a range of 0.01-100 U/ml, and preferably, within a range of 0.1-25 U/ml. (As to definition of the activity of debranching enzymes, please refer to Examples II-6, II-13 and II-14.) The procedure for treatment with a debranching enzyme may be either of the following: The substrate is pre-treated with the debranching enzyme before the α,α-trehalose-producing reaction; or the debranching enzyme is allowed to coexist with the amylase and transferase at any one of the stages during the α,α-trehalose-producing reaction. Preferably, debranching enzymes should be used one or more times at any of the stages, and particularly, should be used one or more times at any of earlier stages. Incidentally, when a thermostable debranching enzyme is used, similar effects can be exhibited by only one time of addition at any one of the stages or earlier stages during the α,α-trehalose-producing reaction.

The produced reaction mixture which contains α,α-trehalose can be purified according to a publicly-known process. For example, the obtained reaction mixture is desalted with an ion-exchange resin; the objective saccharide fraction is-then isolated and crystallized by chromatography using activated charcoal, an ion-exchange resin (HS03 type), cation-exchange resin (Ca type) or the like as a separating material, and by a subsequent condensation to be optionally performed; and finally, α,α-trehalose is yielded within a high purity.

A Gene Coding for the Novel Amylase

The present invention further provides a gene coding for the above novel amylase.

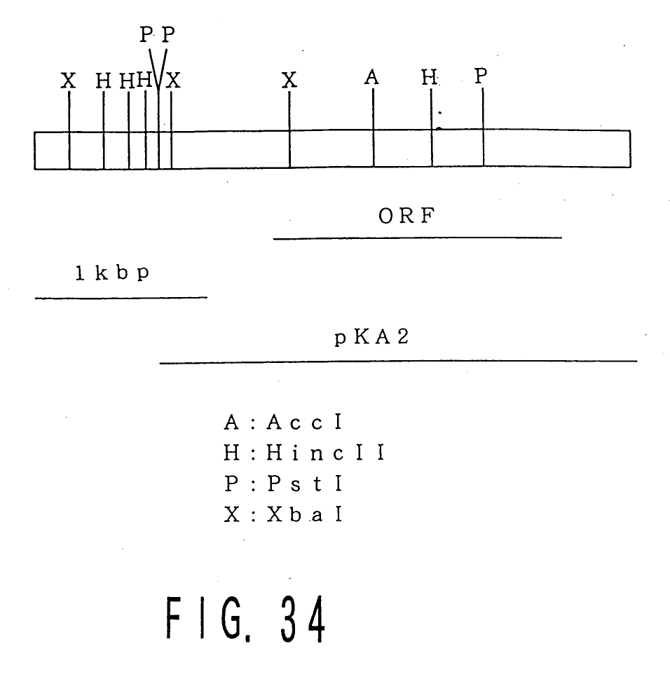

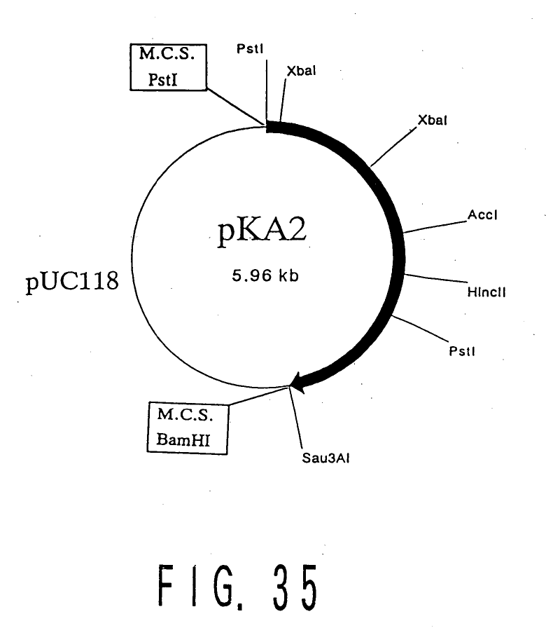

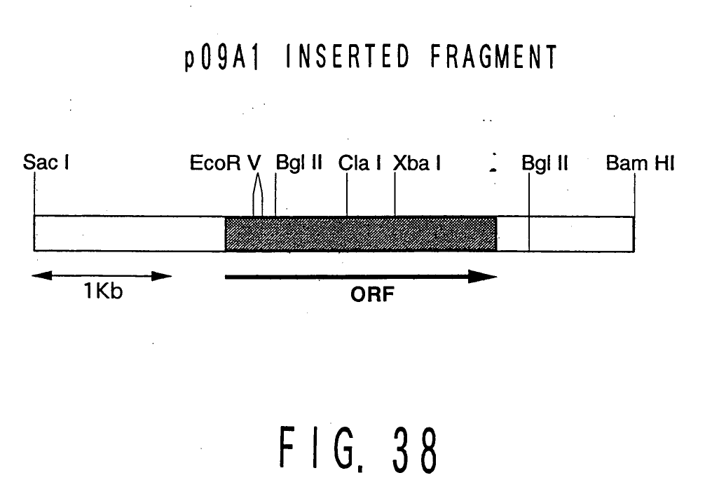

The practical examples of the gene coding for the novel amylase according to the present invention may include the DNA fragments illustrated with restriction maps shown in FIGS. 34 and 38.

These DNA fragments can be derived from archaebacteria belonging to the order Sulfolobales, and preferably, can be isolated from the Sulfolobus solfataricus strain KM1 or the Sulfolobus acidocaldarius strain ATCC 33909 described below. The suitable process for isolation from the Sulfolobus solfataricus strain KM1 or the Sulfolobus acidocaldarius strain ATCC 33909 is illustrated in detail in the examples below.

Examples of the origin from which such a DNA fragments can be obtained may also include the Sulfolobus solfataricus strains DSM 5354, DSM 5833, ATCC 35091 and ATCC 35092; the Sulfolobus acidocaldarius strain ATCC 49426; the Sulfolobus shibatae strain DSM 5389; and the Acidianus brierleyi strain DSM 1651. It is obvious from the following facts that these archaebacteria can be the origins of the DNA fragments according to the present invention: The novel amylase gene derived from the Sulfolobus solfataricus strain KM1 or the Sulfolobus acidocaldarius strain ATCC 33909 forms a hybrid with the chromosome DNA derived from each of those archaebacteria in the below-described hybridization test performed in Example II-24; and further, the characteristics of the enzymes themselves very closely resemble each other as described above. Moreover, the results in the same example suggestively indicate that the novel amylase gene according to the present invention is highly conserved, specifically in archaebacteria belonging to the order Sulfolobales.

The preferable mode for carrying out the present invention provides a DNA fragment comprising a DNA sequence coding for the amino acid sequence shown in Sequence No. 6 or 8 as a suitable example of the gene coding for the novel amylase of the present invention. Further, the base sequence from 642nd base to 2315th base among the base sequence shown in Sequence No. 5 can be listed as a suitable example of the DNA sequence coding for the amino acid sequence shown in Sequence No. 6. The sequence from 1176th base to 2843rd base among the base sequence shown in Sequence No. 7 can be listed as a suitable example of the DNA sequence coding for the amino acid sequence shown in Sequence No. 8.

In general, when given the amino acid sequence of a protein, the base sequence coding therefor can be easily determined by referring to what is called the Codon Table. Therefore, several base sequences which code for the amino acid sequence shown in Sequence No. 6 or 8 can be suitably selected. Accordingly, in the present invention, “the DNA sequence coding for the amino acid shown in Sequence No. 6” implies the DNA sequence comprising the sequence from 642nd base to 2315th base of the base sequence shown in Sequence No. 5; and also, the DNA sequences which comprise the same base sequence as above except that one or more codons are replaced with the codons having a relationship of degeneracy therewith, and which still code for the amino acid shown in Sequence No. 6. Similarly, “the DNA sequence coding for the amino acid shown in Sequence No. 8” implies the DNA sequence comprising the sequence from 1176th base to 2843rd base of the base sequence shown in Sequence No. 7; and also, the DNA sequences which comprise the same base sequence as above except that one or more codons are replaced with the codons having a relationship of degeneracy therewith, and which still code for the amino acid shown in Sequence No. 8.

Further, as described below, the scope of the novel amylase according to the present invention also includes the sequences equivalent to the amino acid sequence shown in Sequence No. 6 or 8. The scope of the DNA fragment according to the present invention, therefore, further includes the base sequences which code for such equivalent sequences.

Moreover, the scope of the novel amylase according to the present invention includes a sequence comprising the amino acid sequence shown in Sequence No. 6 and a Met residue added to the N terminus of this amino acid sequence. Accordingly, the scope of the DNA fragment containing the gene coding for the novel amylase of the present invention also includes the sequence from 639th base to 2315th base of the base sequence shown in Sequence No. 5.

Incidentally, Inventors surveyed the existence of a base sequence homologous to the base sequence shown in Sequence No. 5 or 7 through a data bank on base sequences (EMBL) by using sequence-analyzing software, GENETYX (by Software Development Co.). As a result, Inventors have confirmed that such a base sequence does not exist.

Since the base sequence of the DNA fragment comprising the sequence from 639th or 642nd base to 2315th base of the base sequence shown in Sequence No. 5, and the base sequence of the DNA fragment comprising the sequence from 1176th base to 2843rd base of-the base sequence shown in Sequence No. 7 have been determined, a means for obtaining these DNA fragments is producing them based on a process for polynucleotide synthesis.

Further, these sequences can be obtained by using a process of gene engineering from the above-described archaebacteria belonging to the order Sulfolobales, and preferably, from the Sulfolobus solfataricus strain KM1 or the Sulfolobus acidocaldarius strain ATCC 33909. For example, they can be suitably obtained by a process described in Molecular Cloning: A Laboratory Manual [Sambrook, Mainiatis, et al., published by Cold Spring Harbour Laboratory Press (1989)], and others. The practical method is illustrated in detail in the below-described examples.

Recombinant Novel Amylase

Since the gene coding for the novel amylase is provided as described above, the expressed product from this gene, a recombinant novel amylase, can be obtained according to the present invention.

Suitable examples of the recombinant novel amylase according to the present invention may include an expressed product from the DNA fragment illustrated with the restriction map shown in FIG. 34 or 38.

Also, the suitable examples may include a polypeptide comprising the amino acid sequence shown in Sequence No. 6 or 8 of the Sequence Table, or the equivalent sequence thereof. Here, the term “equivalent sequence” stands for the amino acid sequence which basically has the amino acid sequence shown in Sequence No. 6 or 8; but has undergone insertion, replacement or deletion of some amino acids, or addition of some amino acids to each terminus; and still keeps the activity of the above novel amylase. The state in which the equivalent sequence keeps the activity of the novel amylase means that it keeps an activity sufficient for similar use in similar conditions as compared to the polypeptide having the complete sequence shown in Sequence No. 6 or 8, when the activity is applied in a practical mode for use. Obviously, persons skilled in the art can select and produce such an “equivalent sequence” by referring to the sequences shown in Sequence Nos. 6 and 8 without any special difficulty, since it is revealed in Example II-23 that the same activity is kept in the enzymes derived from the Sulfolobus solfataricus strain KM1 and the Sulfolobus acidocaldarius strain ATCC 33909 though the homology between the amino acid sequences of the novel amylases from these 2 strains is 59% when calculated considering gaps.

Further, the amino acid sequence which comprises the amino acid sequence shown in Sequence No. 6 and a Met residue added to the N terminus of this amino acid sequence is provided according to another mode for carrying out the present invention. The novel amylase of the natural type according to the present invention has the sequence shown in Sequence No. 6. However, as described below, when the novel amylase is obtained from the genetic information of the isolated gene by a recombinant technology using said sequence, the obtained sequence will be found to further have a Met residue in addition to the amino acid sequence shown in Sequence No. 6. Additionally, it is obvious that the obtained sequence has an activity of the novel amylase. Accordingly, the amino acid sequence to which a Met residue is added is also included within the scope of the present invention.

As clarified in Example II-24 below, the DNA fragment having the sequence from 1393th base to 2116th base of the sequence shown in Sequence No. 7 can hybridize with each of the DNA fragments derived from some bacterial strains other than the Sulfolobus acidocaldarius strain ATCC 33909 and the Sulfolobus solfataricus strain KM1 which are the origins of said DNA fragment. Meanwhile, as described above, Inventors have now confirmed the existence of a novel amylase having very close characteristics in those bacterial strains. Further, as revealed in Example II-23 below, the homology between the amino acid sequences of the novel amylases derived from the Sulfolobus solfataricus strain KM1 and the Sulfolobus acidocaldarius strain ATCC 33909 is 59% when calculated considering gaps. It is, therefore, obvious to persons skilled in the art that the activity of the novel amylase can be kept in a sequence which is homologous, to some extent, with the amino acid sequence shown in Sequence No. 6 or 8.

Incidentally, Inventors surveyed the existence of a sequence homologous to the amino acid sequence shown in Sequence No. 6 or 8 through a data bank on amino acid sequences (Swiss prot and NBRF-PFB) by using sequence-analyzing software, GENETYX (by Software Development Co.). As a result, Inventors have confirmed that such a sequence does not exist.

Expression of a Gene Coding for the Novel Amylase

The recombinant novel amylase according to the present invention can be produced in a host cell by transforming the host cell with a DNA molecule, and especially with an expression vector, which can replicate in the host cell, and contains the DNA fragment coding for the novel amylase according to the present invention so as to express the amylase gene.

The present invention, therefore, further provides a DNA molecule, and particularly, an expression vector, which contains a gene coding for the novel amylase according to the present invention. Such a DNA molecule can be obtained by integrating the DNA fragment coding for the novel amylase of the present invention into a vector molecule. According to the preferable mode for carrying out the present invention, the vector is a plasmid.

The DNA molecule according to the present invention can be prepared on the basis of the process described in the aforementioned Molecular Cloning: A Laboratory Manual.

The vector to be used in the present invention can suitably be selected from viruses, plasmids, cosmid vectors, and others considering the type of the host cell to be used. For example, a bacteriophage of λ phage type, a plasmid of pBR or pUC type can be used when the host cell is Escherichia coli; a plasmid of pUB type can be used when the host cell is Bacillus subtilis; and a vector of YEp or YCp type can be used when the host cell is yeast.

The plasmid should preferably contain a selective marker for detection of the transformant, and a drug-resistance marker and an auxotrophy marker can be used as such a selective marker.

Further, the DNA molecule as an expression vector according to the present invention should preferably contain DNA sequences necessary for expression of the novel amylase gene, for example, a transcription-controlling signal, a translation-controlling signal and/or the like such as a promoter, a transcription-initiating signal, a ribosome-binding site, a translation-stopping signal, and a transcription-finishing signal.

Examples of the promoter to be suitably used may include, as well as a promoter functional in the host which contains the insertional fragment, a promoter such as a lactose operon (lac) and a tryptophan operon (trp) for Escherichia coli, a promoter such as an alcohol dehydrogenase gene (ADH), an acid phosphatase gene (PHO), a galactose gene (GAL), and a glyceraldehyde 3-phosphate dehydrogenase gene (GPD) for yeast.

Here, the base sequence comprising the sequence from 1st base to 2691th base of the base sequence shown in Sequence No. 5, and the base sequence comprising the sequence from 1st base to 3600th base of the base sequence shown in Sequence No. 7 are expressed in Escherichia coli to efficiently produce the novel amylase. Accordingly, the DNA sequences shown in Sequence Nos. 5 and 7 are recognized as containing at least sequences necessary for expression in Escherichia coli. It is, therefore, also suitable to use these sequences as they are.

Moreover, when the host cell is Bacillus subtilis or yeast, it will be advantageous to use a secretory vector so as to excrete the recombinant novel amylase outside of the host's body.

In addition to Escherichia coli, Bacillus subtilis, yeast, and advanced eukaryotes, can be used as a host cell. Microorganisms belonging to the genus Bacillus such as Bacillus subtilis are suitably used. Some strains belonging to this genus are known to excrete a protein outside of the bacterial body in a large amount. Therefore, a large amount of the recombinant novel amylase can be excreted in the culture medium by using a secretory vector. This is preferable because the purification from the supernatant of the culture will be easy. Further, some strains belonging to the genus Bacillus are known to excrete a very little amount of protease outside of the bacterial body. It is preferable to use such strains because the recombinant novel amylase can be efficiently produced thereby. Moreover, it will be very advantageous to select a microorganism which does not produce glucoamylase and to use it as a host cell, because the recombinant novel amylase of the present invention which is obtained as a cell extract or a simply-purified crude enzyme can be directly used for the below-described production of α,α-trehalose.

The recombinant novel amylase produced by the aforementioned transformant can be obtained as follows: At first, the above-described host cell is cultivated under proper conditions; the bacterial bodies are collected from the resultant culture by a publicly-known method, for example, by centrifugation, and suspended in a proper buffer solution; the bacterial bodies are then crushed by freeze thawing, an ultrasonic treatment, grinding and/or the like; and the resultant is centrifuged or filtrated to obtain a cell extract containing the recombinant novel amylase.

Purification of the recombinant novel amylase existing in the cell extract can be performed by a proper combination of publicly-known processes for isolation and purification. Examples of the processes may include a process utilizing a difference in thermostability, such as a heat treatment; a process utilizing a difference in solubility, such as salt precipitation and solvent precipitation, a process utilizing a difference in molecular weight, such as dialysis, ultrafiltration, gel filtration and SDS-Polyacrylamide gel electrophoresis; a process utilizing a difference in electric charge, such as ion exchange chromatography; a process utilizing specific affinity, such as affinity chromatography; a process utilizing a difference in hydrophobicity, such as hydrophobic chromatography and reversed phase chromatography; and further, a process utilizing a difference in isoelectric point, such as isoelectric focusing. Since the recombinant novel amylase is thermostable, the purification can be very easily performed using heat treatment, by which proteins in the host can be denatured and made into precipitation suitable for removal.

Production of α,α-Trehalose Using the Recombinants

The present invention further provides a process for producing α,α-trehalose by using the above recombinant novel amylase and the aforementioned recombinant novel transferase.

According to the preferable mode for producing α,α-trehalose, the recombinant novel amylase and the recombinant transferase of the present invention may be mixed and put into contact at the same time with glucide such as starch, starch hydrolysate and maltooligosaccharide. Also, it is preferable to substitute either of the recombinant transferase and the recombinant novel amylase with a corresponding enzyme derived from nature.

The concentration of glucide such as starch, starch hydrolysate and maltooligosaccharide should be suitably selected within the range in which the glucide to be used is dissolved, considering the specific activities of the present enzymes, the reaction temperature and others. A range of 0.5-70% is ordinary, and a range of 5-40% is preferable. The reaction temperature and pH condition in the reaction of the glucide with the enzymes should be optimum for the recombinant novel amylase and the recombinant novel transferase. Accordingly, the reaction is performed ordinarily at 50-85° C. and pH 3.5-8, approximately, and more preferably, at 60-75° C. and pH 4.5-6.0.

Additionally, when the glucide to be used is starch, starch hydrolysate, or the like having a high polymerization degree, the production of α,α-trehalose can be further promoted by using another endotype liquefying amylase together as a supplement. For example, enzymes derived from bacteria belonging to the genus Bacillus, fungi belonging to the genus Aspergillus, and plants such as malt, and others can be used as such an endotype liquefying amylase. The debranching enzyme to be used may be pullulanase derived from bacteria belonging to the genus Bacillus, Klebsiella or the like, isoamylase derived from bacteria belonging to the genus Pseudomonas, or the like. Further, these enzymes may be used in combination.

However, the addition of an excess amount of an endotype liquefying amylase will cause production of glucose and maltose which the novel transferase will not act on. Similarly, the addition of an excess amount of pullulanase will cause a decrease in solubility of the substrate due to cleavage of the 1,6-linkage, and lead to production of a highly-viscous and insoluble substance which can not be utilized. For that reason, the amounts of endotype liquefying amylase and pullulanase should be controlled so as not to produce excessive glucose, maltose, or an insoluble substance.

Any of the procedures may be employed when pullulanase is used, for example, pre-treating the substrate with pullulanase, or putting pullulanase into coexistence together with the recombinant novel amylase and the recombinant novel transferase at any one of the stages during the α,α-trehalose-producing reaction.

The produced reaction mixture which contains α,α-trehalose can be purified according to a publicly-known process. For example, the obtained reaction mixture is desalted with an ion-exchange resin; the objective saccharide fraction is then isolated and crystallized by chromatography using activated charcoal, an ion-exchange resin (HS0₃ type), cation-exchange resin (Ca type) or the like as a separating material, and by a subsequent condensation to be optionally performed; and finally, α,α-trehalose is yielded within a high purity.

The present invention will be further illustrated in detail with practical examples below, though, needless to say, the scope of the present invention is not limited to within those examples.

### EXAMPLE I-1

### Glucosyltrehalose-Producing Activities of Archaebacteria

The bacterial strains listed in Table 3 below were examined for glucosyltrehalose-producing activity. The examination was performed as follows: The cultivated bacterial bodies of each strain was crushed by an ultrasonic treatment and centrifuged; the substrate, maltotriose, was added to the supernatant so that the final concentration would be 10%; the mixture was then put into a reaction at 60° C. for 24 hours; after that, the reaction was stopped by a heat-treatment at 100° C. for 5 min.; and the glucosyltrehalose thus produced was subjected to a measurement according to the HPLC analysis under the below-described conditions.

| | |
|---|---|
| Column: | TOSOH TSK-gel Amide-80 (4.6 × 250 mm) |
| Solvent: | 75% acetonitrile |
| Flow rate: | 1.0 ml/min. |
| Temperature: | Room temperature |
| Detector: | Refractive Index Detector |

The enzyme activities were expressed with such a unit as 1 Unit equals the activity of converting maltotriose into 1 μmol of glucosyltrehalose per hour. Incidentally, in Table 3, the activity was expressed in terms of units per one gram of bacterial cell (Units/g-cell).

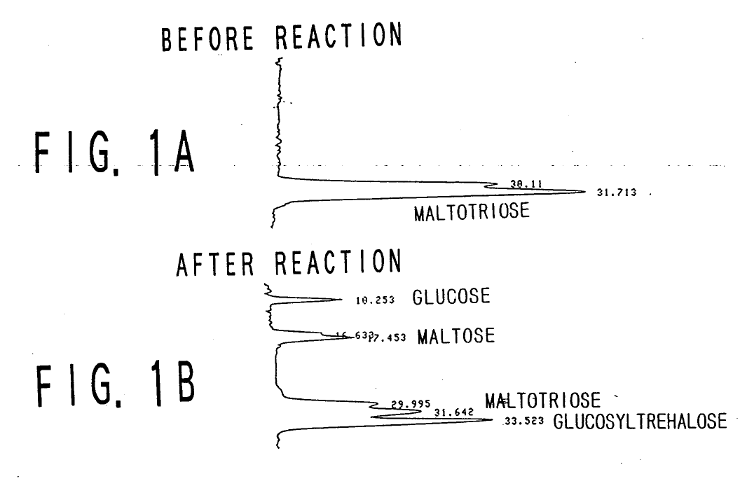

FIG. 1(B) is the HPLC chart obtained herein. As is recognized from the figure, the principal reaction product appeared slightly behind the non-reacted substrate in the HPLC chart as one peak without any anomer. The aliquot of this principal reaction product through TSK-gel Amide-80 HPLC column was subjected to ¹H-NMR analysis and ¹³C-NMR analysis, and was confirmed to be glucosyltrehalose. The chemical formula is as follows.

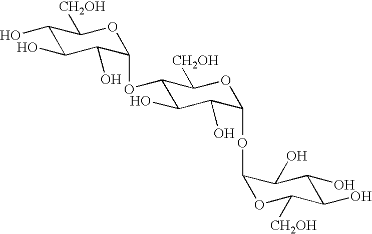

Consequently, each of the cell extracts from the bacterial strains belonging to the order Sulfolobales has a glucosyltrehalose-producing activity, namely, the transferase activity as the enzyme of the present invention.

TABLE 3

| | | Enzyme activity |
|---|---|---|
| Strain | | (Uints/g-cell) |
| Sulfolobus solfataricus | ATCC 35091 | 6.8 |
| | ATCC 35092 | 6.0 |
| | DSM 5354 | 13.0 |
| | DSM 5833 | 5.6 |
| | KM1 | 13.5 |
| Sulfolobus acidocaldarius | ATCC 33909 | 13.0 |
| | ATCC 49426 | 2.4 |
| Sulfolobus shibatae | DSM 5389 | 12.0 |
| Acidianus brierleyi | DSM 1651 | 6.7 |

### EXAMPLE I-2

### Purification of the Present Transferase Derived from the Sulfolobus solfataricus Strain KM1

The Sulfolobus solfataricus strain KM1 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 3.3 g/liter.

Two hundred grams of the bacterial cells obtained above were suspended in 400 ml of a 50 mM sodium acetate buffer solution (pH 5.5) containing 5 mM of EDTA, and subjected to an ultrasonic treatment for bacteriolysis at 0° C. for 15 min. The resultant was then centrifuged to obtain a supernatant, and ammonium sulfate was added to the supernatant so as to be 60% saturation.

The precipitate obtained by centrifugation was dissolved in a 50 mM sodium acetate buffer solution (pH 5.5) containing 1 M of ammonium sulfate and 5 mM of EDTA, and applied to a hydrophobic chromatography using the TOSOH TSK-gel Phenyl-TOYOPEARL 650S column (volume: 800 ml) equilibrated with the same buffer solution as above. The column was then washed with the same buffer solution, and the objective transferase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM sodium acetate-buffer solution (pH 5.5).

Next, the resultant was subjected to ion-exchange chromatography using the TOSOH TSK-,gel DEAE-TOYOPEARL 650S column (volume: 300 ml) equilibrated with the same buffer solution. The column was then washed with the same buffer solution, and the objective transferase was eluted with 900 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Subsequent to that, the desalted and concentrated solution thus obtained was subjected to gel filtration chromatography using the Pharmacia HiLoad 16/60 Superdex 200pg column, and the objective transferase was eluted with the same buffer solution. The fractions exhibiting the activity were concentrated using an ultrafiltration membrane (critical molecular weight: 13,000), and subsequently, washed and desalted with a 50 mM sodium acetate buffer solution (pH 5.5).

Next, ammonium sulfate was dissolved in the desalted and concentrated solution thus obtained so that the concentration of ammonium sulfate would be 1 M. The resultant was then subjected to hydrophobic chromatography using TOSOH TSK-gel Phenyl-5PW HPLC column equilibrated with the same buffer solution. The column was then washed with the same buffer solution, and the objective transferase was eluted with 30 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM sodium acetate buffer solution (pH 5.0).

Further, the resultant was subjected to ion-exchange chromatography using the TOSOH TSK-gel DEAE 5PW HPLC column equilibrated with the same buffer solution. The column was then washed with the same buffer solution, and the objective transferase was eluted with 30 ml of sodium chloride solution at a linear concentration gradient from 0 M to 0.3 M. The fractions exhibiting the activity were concentrated using an ultrafiltration membrane (critical molecular weight: 13,000).

Finally, Native Polyacrylamide gel electrophoresis, SDS-Polyacrylamide gel electrophoresis and isoelectric focusing were performed to obtain the purified enzyme which appeared as single band.

Incidentally, the activity was measured in the same manner as in Example I-1.

Total enzyme activity, total protein and specific activity at each of the purification steps are shown in Table 4 below.

TABLE 4

| | Total enzyme | Total protein | Specific activity | Yield | Purity |
|---|---|---|---|---|---|
| Purified fraction | activity (units) | (mg) | (units/mg) | (%) | (fold) |
| Crude extract | 653 | 17000 | 0.038 | 100 | 1 |
| 60% saturated (NH₄)₂SO₄ | 625 | 15000 | 0.04 | 95.7 | 1.1 |
| precipitation | | | | | |
| Phenyl | 83 | 533 | 0.16 | 12.7 | 4.2 |
| DEAE | 150 | 31 | 4.90 | 23.0 | 129 |
| Gel-permeation | 111 | 2 | 55.7 | 17.0 | 1466 |
| Phenyl rechromatography | 48 | 0.17 | 277 | 7.4 | 7289 |
| DEAE rechromatography | 30 | 0.05 | 598 | 4.6 | 15737 |

### EXAMPLE I-3

### Purification of the Present Transferase Derived from Sulfolobus solfataricus strain DSM 5833

The Sulfolobus solfataricus strain DSM 5833 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 1.7 g/liter.

Fifty six grams of the bacterial cells obtained above were suspended in 100 ml of a 50 mM sodium acetate buffer solution (pH 5.5) containing 5 mM of EDTA, and subjected to an ultrasonic treatment for bacteriolysis at 0° C. for 15 min. The resultant was then centrifuged to obtain a supernatant.

Next, ammonium sulfate was dissolved in the supernatant so that the concentration of ammonium sulfate would be 1 M. The resultant was then subjected to hydrophobic chromatography using TOSOH TSK-gel Phenyl-TOYOPEARL 650S column (volume: 200 ml) equilibrated with a 50 mM sodium acetate bufier solution (pH 5.5.) containing.1 M of sodium. sulfate and 5 mM of EDTA. The column was then washed with the same buffer solution, and the objective transferase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM Tris-HCl buffer solution (pH 7.5).

Subsequent to that, the resultant was subjected to ion-exchange chromatography using the TOSOH TSK-gel DEAE-TOYOPEARL 650S column (volume: 300 ml) equilibrated with the same buffer solution. The column was then washed with the same buffer solution, and the objective transferase was eluted with 900 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Next, ammonium sulfate was dissolved in the desalted and concentrated solution thus obtained so that the concentration of ammonium sulfate would be 1 M. The resultant was then subjected to hydrophobic chromatography using TOSOH TSK-gel Phenyl-TOYOPEARL 650S column (volume: 200 ml) equilibrated with the same buffer solution. The column was then washed with the same buffer solution, and the objective transferase was eluted with 600 ml of ammonium sulfate solution at a linear concentration gradient from 1 M to 0 M. The fractions exhibiting the activity were concentrated using an ulttafiltration membrane (critical molecular weight: 13,000), and subsequently, washed and desalted with a 50 mM sodium acetate buffer solution (pH 5.5) containing 0.15 M of sodium chloride and 5 mM of EDTA.

Further, the desalted and concentrated solution thus obtained was subjected to gel filtration chromatography using the Pharmacia HiLoad 16/60 Superdex 200pg column, and the objective transferase was eluted with the same buffer solution. The fractions exhibiting the activity were concentrated using an ultrafiltration membrane (critical molecular weight: 13,000), and subsequently, dialyzed with a 25 mM Bis-Tris-HCl buffer solution (pH 6.7).

Next, the resultant was subjected to a chromatofocusing using the Pharmacia Mono P HR/5/20 column equilibrated with the same buffer solution. Immediately after the sample was injected, the objective transferase was eluted with 10% polybuf fer 74-HCl (pH 5.0; manufactured by Pharmacia Co.). The fractions exhibiting the activity were concentrated using an ultrafiltration membrane (critical molecular weight: 13,000), and subsequently, dialyzed with a 25 mM Bis-Tris-HCl buffer solution (pH 6.7).

Further, another chromatofocusing was performed under the same conditions, and the objective transferase was eluted. The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA. Finally, Native polyacrylamide gel electrophoresis, SDS-polyacrylamide gel electrophoresis and isoelectric focusing were performed to obtain the purified enzyme which appeared as single band.

Incidentally, the activity was measured in the same manner as in Example I-1.

Total enzyme activity, total protein and specific activity at each of the purification steps are shown in Table 5 below.

TABLE 5

| | Total | | | | |
|---|---|---|---|---|---|
| | enzyme | Total | Specific | | |
| | activity | protein | activity | | Purity |
| Purified fraction | (units) | (mg) | (units/mg) | Yield (%) | (fold) |
| Crude extract | 541 | 10000 | 0.06 | 100 | 1 |
| Phenyl | 1039 | 988 | 1.05 | 192 | 19 |
| DEAE | 383 | 147 | 2.60 | 70.7 | 47 |
| Pheny | 248 | 49.5 | 5.00 | 45.8 | 91 |
| rechromatography | | | | | |
| Gel-permeation | 196 | 3.69 | 53.0 | 36.1 | 964 |
| Mono P | 92 | 0.32 | 287 | 17.0 | 5218 |
| Mono P | 64 | 0.13 | 494 | 11.9 | 8982 |
| rechromatography | | | | | |

### EXAMPLE I-4

### Purification of the Present Transferase Derived from the Sulfolobus acidocaldarius Strain ATCC 33909

The Sulfolobus acidocaldarius strain ATCC 33909 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 2.9 g/liter.

Ninety two and a half grams of the bacterial cells obtained above were suspended in 200 ml of a 50 mM sodium acetate buffer solution (pH 5.5) containing 5 mM of EDTA, and subjected to an ultrasonic treatment for bacteriolysis at 0° C. for 15 min. The resultant was then centrifuged to obtain a supernatant.

Next, ammonium sulfate was dissolved in the supernatant so that the concentration of ammonium sulfate would be 1 M. The resultant was then subjected to hydrophobic chromatography using TOSOH TSK-gel Phenyl-TOYOPEARL 650S column (volume: 400 ml) equilibrated with a 50 mM sodium acetate buffer solution (pH 5.5) containing 1 M of sodium sulfate and 5 mM EDTA. The column was then washed with the same buffer solution, and the objective transferase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM Tris-HCl buffer solution (pH 7.5).

Subsequent to that, the resultant was subjected to ion-exchange chromatography using the TOSOH TSK-gel DEAE-TOYOPEARL 650S column (volume: 300 ml) equilibrated with the same buffer solution. The column was then washed with the same buffer solution, and the objective transferase was eluted with 900 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Next, ammonium sulfate was dissolved in the desalted and concentrated solution thus obtained so that the concentration of ammonium sulfate would be 1 M. The resultant was then subjected to hydrophobic chromatography using TOSOH TSK-gel Phenyl-TOYOPEARL 650S column (volume: 200 ml) equilibrated with the same buffer solution. The column was then washed with the same buffer solution, and the objective transferase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 50 mM. sodium acetate buffer solution (pH 5.5) containing 0.15 M of sodium chloride and 5 mM EDTA.

Further, the desalted and concentrated solution thus obtained was subjected to gel filtration chromatography using the Pharmacia HiLoad 16/60 Superdex 200pg column, and the objective transferase was eluted with the same buffer solution. The fractions exhibiting the activity were concentrated using an ultrafiltration membrane (critical molecular weight: 13,000), and subsequently, dialyzed with a 25 mM Bis-Tris-HCl buffer solution (pH 6.7).

Next, the resultant was subjected to a chromatofocusing using the Pharmacia Mono P HR/5/20 column equilibrated with the same buffer solution. Immediately after the sample was injected, the objective transferase was eluted with 10% polybuf fer 74-HCl (pH 5.0; manufactured by Pharmacia Co.). The fractions exhibiting the activity were concentrated using an ultrafiltration membrane (critical molecular weight: 13,000), and subsequently, dialyzed with a 25 mM Bis-Tris-HCl buffer solution (pH 6.7).

Further, another chromatofocusing was performed under the same conditions, and the objective transferase was eluted. The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Finally, Native polyacrylamide gel electrophoresis, SDS-polyacrylamide gel electrophoresis and isoelectric focusing were performed to obtain the purified enzyme which appeared as single band.

Incidentally, the activity was measured in the same manner as in Example I-1.

Total enzyme activity, total protein and specific activity at each of the purification steps are shown in Table 6 below.

TABLE 6

| | Total | | | | |
|---|---|---|---|---|---|
| | enzyme | Total | Specific | | |
| | activity | protein | activity | | Purity |
| Purified fraction | (units) | (mg) | (units/mg) | Yield (%) | (fold) |
| Crude extract | 912 | 38000 | 0.24 | 100 | 1 |
| Phenyl | 559 | 660 | 0.85 | 61.3 | 3.5 |
| DEAE | 806 | 150 | 5.40 | 88.4 | 23 |
| Phenyl | 636 | 35.1 | 18.1 | 69.7 | 75 |
| rechromatography | | | | | |
| Gel-permeation | 280 | 2.68 | 104 | 30.7 | 433 |
| Mono P | 129 | 0.35 | 411 | 13.8 | 1713 |
| Mono P | 86.9 | 0.24 | 362 | 9.5 | 1508 |
| rechromatography | | | | | |

### EXAMPLE I-5

### Purification of the Present Transferase Derived from the Acidianus brierleyi Strain DSM 1651

The Acidianus brierleyi strain DSM 1651 was cultivated at 70° C. for 3 days in the culture medium which is identified as No. 150 in Catalogue of Strains 5th edition (1993) published by Deutsche Sammlung von Mikroorganismen und Zellkulturen GmbH (DSM). The cultivated bacteria was collected by centrifugation and stored at −80° C. The yield of the bacterial cell was 0.6 g/liter.

Twelve grams of the bacterial cells obtained above were suspended in 120 ml of a 50 mM sodium acetate buffer solution (pH 5.5) containing 5 mM of EDTA, and subjected to an ultrasonic treatment for bacteriolysis at 0° C. for 1.5 min. The resultant was then centrifuged to obtain a supernatant.

Next, ammonium sulfate was dissolved in the supernatant so that the concentration of ammonium sulfate would be 1 M. The resultant was then subjected to hydrophobic chromatography using TOSOH TSK-gel Phenyl-TOYOPEARL 650S column (volume: 200 ml) equilibrated with a 50 mM sodium acetate buffer solution (pH 5.5) containing 1 M of sodium sulfate and 5 mM of EDTA. The column was then washed with the same buffer solution, and the objective transferase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and. desalted with a 10 mM Tris-HCl buffer solution (pH 7.5).

Subsequent to that, the resultant was subjected to ion-exchange chromatography using the TOSOH TSK-gel DEAE-TOYOPEARL 650S column (volume: 300 ml) equilibrated with the same buffer solution. The column was then washed with the same buffer solution, and the objective transferase was eluted with 900 ml of sodium chloride solution at a linear concentration gradient from 0 M to 0.3 M. . The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Further, the desalted and concentrated solution thus obtained was subjected to gel filtration chromatography using the Pharmacia HiLoad 16/60 Superdex 200pg column, and the objective transferase was eluted with the same buffer solution. The fractions exhibiting the activity were concentrated using an ultrafiltration membrane (critical molecular weight: 13,000), and subsequently, dialyzed with a 25 mM Bis-Tris-HCl buffer solution (pH 6.7).

Next, the resultant was subjected to a chromatofocusing using the Pharmacia Mono P HR/5/20 column equilibrated with the same buffer solution. Immediately after the sample. was injected, the objective transferase was eluted with 10% polybuffer 74-HCl (pH 5.0; manufactured by Pharmacia Co.). The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Finally, Native Polyacrylamide gel electrophoresis, SDS-Polyacrylamide gel electrophoresis and isoelectric focusing were performed to obtain the purified enzyme which appeared as single band.

Incidentally, the activity was measured in the same manner as in Example I-1.

Total enzyme activity, total protein and specific activity at each of the purification steps are shown in Table 7 below.

TABLE 7

| | Total enzyme | Total | Specific | | |
|---|---|---|---|---|---|
| | activity | protein | activity | Yield | Purity |
| Purified fraction | (units) | (mg) | (units/mg) | (%) | (fold) |
| Crude extract | 310 | 264 | 1.17 | 100 | 1 |
| Phenyl | 176 | 19.2 | 9.20 | 56.9 | 7.9 |
| DEAE | 70 | 5.02 | 13.8 | 22.5 | 12 |
| Gel-permeation | 54 | 0.18 | 298 | 17.3 | 255 |
| Mono P | 27 | 0.07 | 378 | 8.6 | 323 |

### EXAMPLE I-6

### Examination of the Present Transferase for Various Characteristics

The purified enzyme obtained in Example I-2 was examined for enzymatic characteristics.

(1) Molecular Weight

The molecular weight of the purified enzyme in its native state was measured by gel filtration chromatography using the Pharmacia HiLoad 16/60 Superdex 200pg column. Marker proteins having molecular weights of 200,000, 97,400, 68,000, 43,000, 29,000, 18,400 and 14,300, respectively, were used.

As a result, the molecular weight of the transferase was estimated at 54,000.

Meanwhile, the molecular weight was also measured by SDS-polyacrylamide gel electrophoresis (gel concentration; 6%). Marker proteins having molecular weights of 200,000, 116,300, 97,400, 66,300, 55,400, 36,500, 31,000, 21,500 and 14,400, respectively, were used.

As a result, the molecular weight of the transferase was estimated at 76,000.

The difference between molecular weight values measured by gel filtration chromatography and SDS-Polyacrylamide gel electrophoresis may be attributed to a certain interaction which may be generated between the packed material of the gel filtration column and proteins. Accordingly, the molecular weight value estimated by gel filtration does not necessarily represent the molecular weight of the present enzyme in its native state.

(2) Isoelectric Point

The isoelectric point was found to be pH 6.1 by agarose gel isoelectric focusing.

(3) Stability

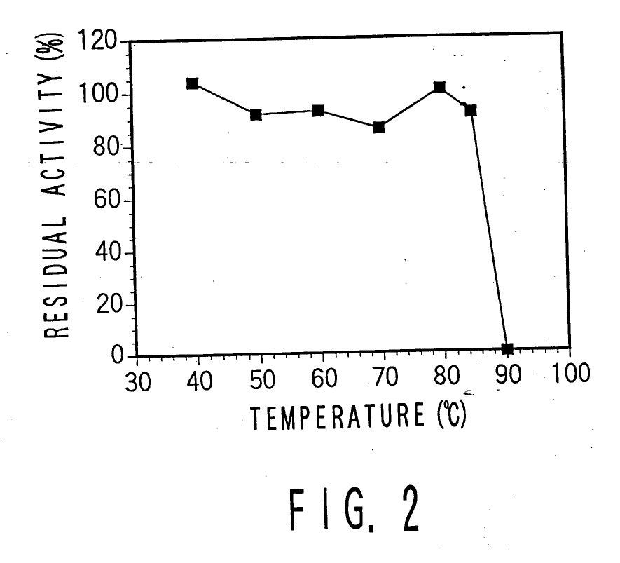

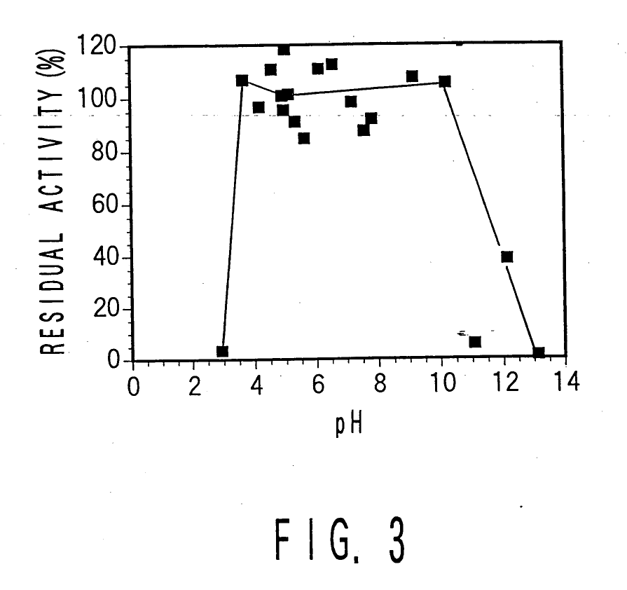

The stability changes of the obtained enzyme according to temperature and pH value are shown in FIGS. 2 and 3, respectively. In measurement, a glycine-HCl buffer solution was used in a pH range of 3-5, and similarly, a sodium acetate buffer solution in a pH range of 4-6, a sodium phosphate buffer solution in a pH range of 5-8, a Tris-HCl buffer solution in a pH range of 8-9, a sodium bicarbonate buffer solution in a pH range of 9-10, and a KCl-NaOH buffer solution in a pH range of 11-13, respectively, were also used.

The present enzyme was stable throughout the treatment at 85° C. for 6 hours, and also, was stable throughout the treatment at pH 4.0-10.0 and room temperature for 6 hours.

(4) Reactivity

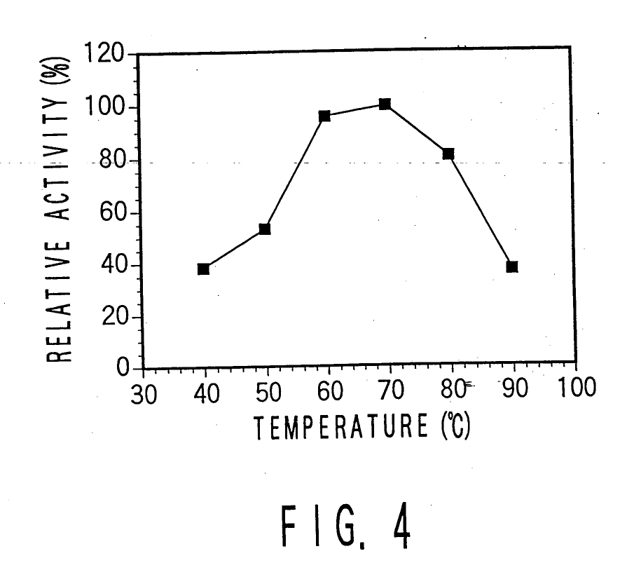

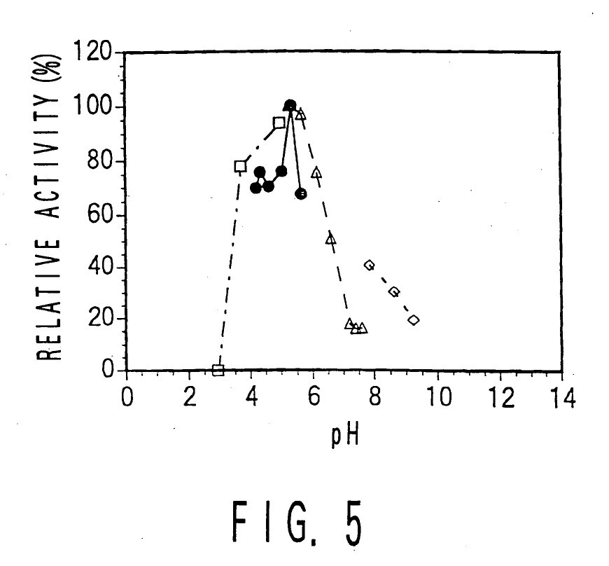

As to the obtained enzyme, reactivity of at various temperatures and reactivity at various pH are shown in FIGS. 4 and 5, respectively. In measurement, a glycine-HCl buffer solution was used in a pH range of 3-5 (□), similarly, a sodium acetate buffer solution in a pH range of 4-5.5 (●), a sodium phosphate buffer solution in a pH range of 5-7.5 (Δ), and a Tris-HCl buffer solution in a pH range of 8-9 (⋄), respectively, were also used.

The optimum reaction temperature of the present enzyme is within 60-80° C., approximately, and the optimum reaction pH of the present enzyme is within 5.0-6.0, approximately.

(5) Influence of Various Activators and Inhibitors

The influence of each substance listed in Table 8, such as an activating effect or inhibitory effect, was evaluated using similar activity-measuring method to that in Example I-1. Specifically, the listed substances were individually added together with the substrate to the same reaction system as that in the method for measuring glucosyltrehalose-producing activity employed in Example I-1. As a result, copper ion and SDS were found to have inhibitory effects. Though many glucide-relating enzymes have been found to be activated with calcium ion, the present enzyme would not be activated with calcium ion.

TABLE 8

| | Concen- | Residual |
|---|---|---|
| | tration | activity |
| Activator/Inhibitor | (mM) | (%) |
| Control (not added) | | 100.0 |
| CaCl₂ | 5 | 93.6 |
| MgCl₂ | 5 | 111.3 |
| MnCl₂ | 5 | 74.2 |
| CuSO₄ | 5 | 0.0 |
| CoCl₂ | 5 | 88.5 |
| FeSO₄ | 5 | 108.3 |
| FeCl₃ | 5 | 90.0 |
| AgNO₃ | 5 | 121.0 |
| EDTA | 5 | 96.8 |
| 2-Mercaptoethanol | 5 | 100.3 |
| Dithiothreitol | 5 | 84.5 |
| SDS | 5 | 0.0 |
| Glucose | 0.5 | 107.3 |
| Trehalose | 0.5 | 107.8 |
| Maltotetraose | 0.5 | 97.4 |
| Malatopentaose | 0.5 | 101.9 |
| Maltohexaose | 0.5 | 91.0 |
| Maltoheptaose | 0.5 | 93.5 |

(6) Substrate Specificity

It was investigated whether or not the present enzyme acts on each of the substrates listed in Table 9 below to produce its α-1,α-1-transferred isomer. Here, the activity measurement was performed in the same manner as in Example I-1.

TABLE 9

| Substrate | Reactivity |
|---|---|
| Glucose | − |
| Maltose | − |
| Maltotriose (G3) | + |
| Maltotetraose (G4) | ++ |
| Malotopentaose (G5) | ++ |
| Maltohexaose (G6) | ++ |
| Maltoheptaose (G7) | ++ |
| Isomaltotriose | − |
| Isomaltotetraose | − |
| Isomaltopentaose | − |
| Panose | − |

As a result, the present enzyme was found to produce trehaloseoligosaccharides from the substrates of maltotriose (G3) -maltoheptaose (G7). Meanwhile, the present enzyme did not act on any of isomaltotriose, isomaltotetraose, isomaltopentaose or panose, which have α-1,6 linkages at 1st to 4th linkages from the reducing end or have the α-1,6 linkage at 2nd linkage from the reducing end.

Incidentally, each of the purified enzymes which were obtained in Examples I-3-I-5 and derived from the Sulfolobus solfataricus strain DSM 5833, the Sulfolobus acidocaldarius strain ATCC 33909, and the Acidianus brierleyi strain DSM 1651, respectively, was examined for enzymatic characteristics by using similar manner. The results are shown in Table 1 above.

### EXAMPLE I-7

### Production of Glucosyltrehalose and Maltooligosyltrehalose from Maltooligosaccharides

As the substrates, maltotriose (G3)-maltoheptaose (G7) were used in a concentration of 100 mM. The purified enzyme obtained in Example I-2 was then allowed to act on each of the above substrates in an amount of 13.5 Units/ml (in terms of the enzyme activity when the substrate is maltotriose) to produce a corresponding α-1,α-1-transferred isomer. Each product was analyzed by the method in Example I-1, and investigated its yield and enzyme activity. The results was shown in Table 10 below. Incidentally, in Table 10, each enzymatic activity value was expressed with such a unit as 1 Unit equals the activity of converting the maltooligosaccharide into-i pmol of corresponding α-1,α-1-transferred isomer per hour.

TABLE 10

| | Enzyme activity | Yield |
|---|---|---|
| Substrate | (units/ml) | (%) |
| Maltotriose (G3) | 13.5 | 44.6 |
| Maltotetraose (G4) | 76.3 | 73.1 |
| Maltopentaose (G5) | 111.3 | 68.5 |
| Maltohexaose (G6) | 100.9 | 63.5 |
| Maltoheptaose (G7) | 70.5 | 68.7 |

As is shown in Table 10, the enzyme activity was highest when the substrate was G5, which exhibited approximately 8 times as much activity as G3. Further, the yield was 44.6% in G3, while 63.5-73.1% in G4 or larger.

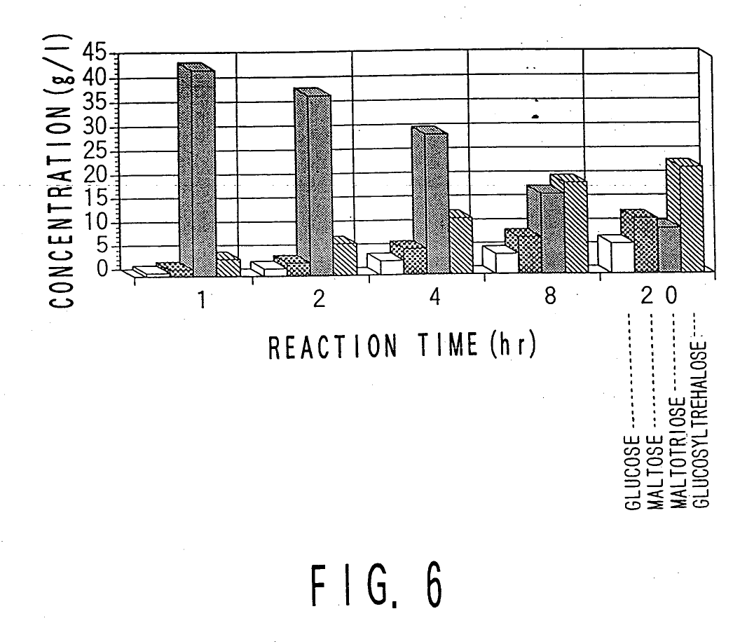

Additionally, the composition of each product which was obtained from G3, G4 or G5 assigned for a substrate was investigated. The results are shown in FIGS. 6-8, respectively.

Specifically, when maltotriose was used as a substrate, glucosyltrehalose was produced as a product in the principal reaction, and in addition, equal moles of maltose and glucose were produced as products in the side reaction.

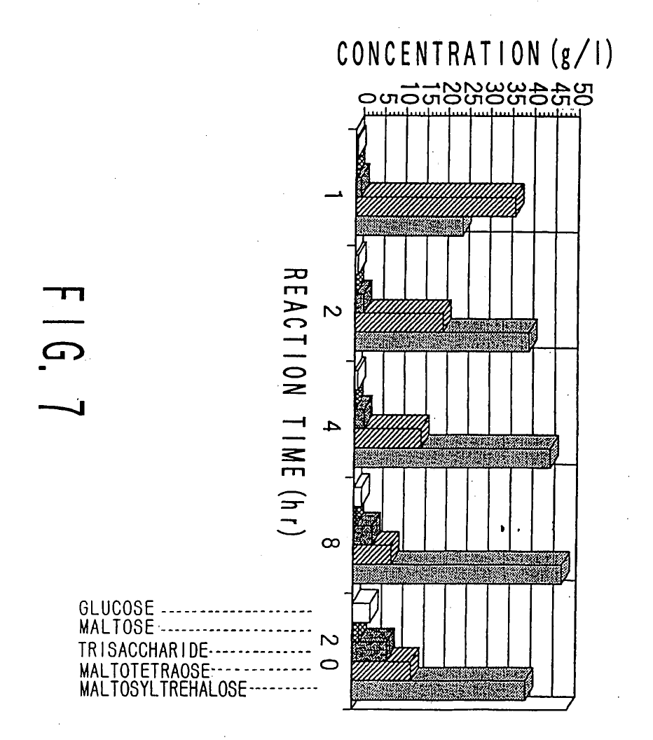

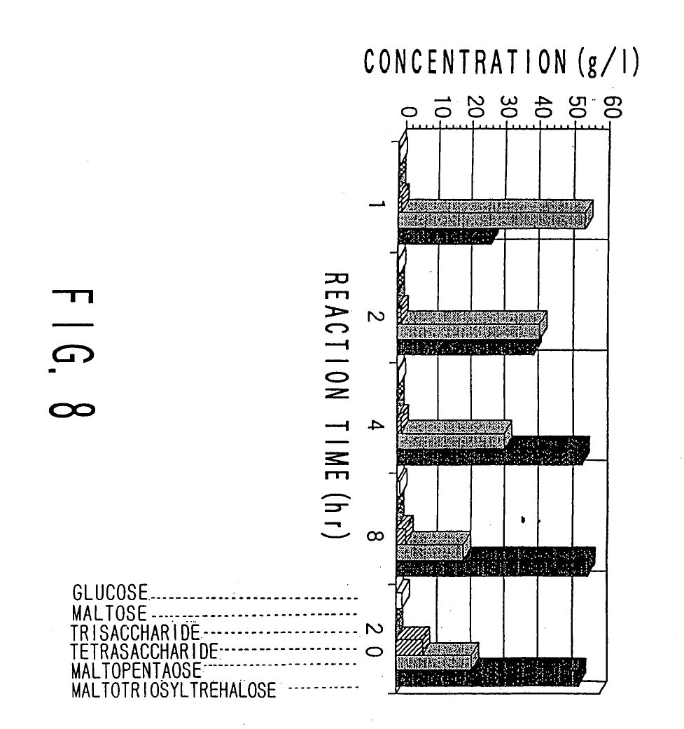

When the substrate was a saccharide having a polymerization degree, n, which is equal to or higher than that of maltotetraose, the product in the principal reaction was a saccharide, of which the polymerization degree is n, and the glucose residue at the reducing end is α-1,α-1-linked. And in addition, equal moles of glucose and a saccharide having a polymerization degree of n−1 were produced in the side reaction. Additionally, when the reaction further progressed in these saccharides, the saccharide having a polymerization degree of n−1 secondarily underwent the reactions similar to the above. (Incidentally, in FIGS. 7 and 8, saccharides indicated as trisaccharide and tetrasaccharide include non-reacted maltotriose and maltotetraose, respectively, and also include the saccharides, of which the linkage at an end is α-1,α-1, were produced when the reactions similar to the above progressed secondarily.) Meanwhile, the production of such a saccharide as having a polymerization degree of n+1 or higher, namely, an intermolecularly-transferred isomer, was not detected. Incidentally, hydrolysis as the side reaction occurred less frequently when the chain length was the same as or longer than that of G4.

The trisaccharide, the tetrasaccharide and the pentasaccharide which are the principal products from the substrates, G3, G4 and G5, respectively, were sampled by the TSK-gel Amide-80 HPLC column as examples of principal products in the above, and analyzed by ¹H-NMR and ¹³C-NMR. As a result, it was found that the glucose residue at the reducing end of each saccharide was α-1,α-1-linked, and those saccharides were recognized as glucosyltrehalose (α-D-maltosyl α-D-glucopyranoside), maltosyltrehalose (α-D-maltotriosyl α-D-glucopyranoside), and maltotriosyl-trehalose (α-D-maltotetraosyl α-D-glucopyranoside), respectively. The chemical formulae of these saccharides are as follows.

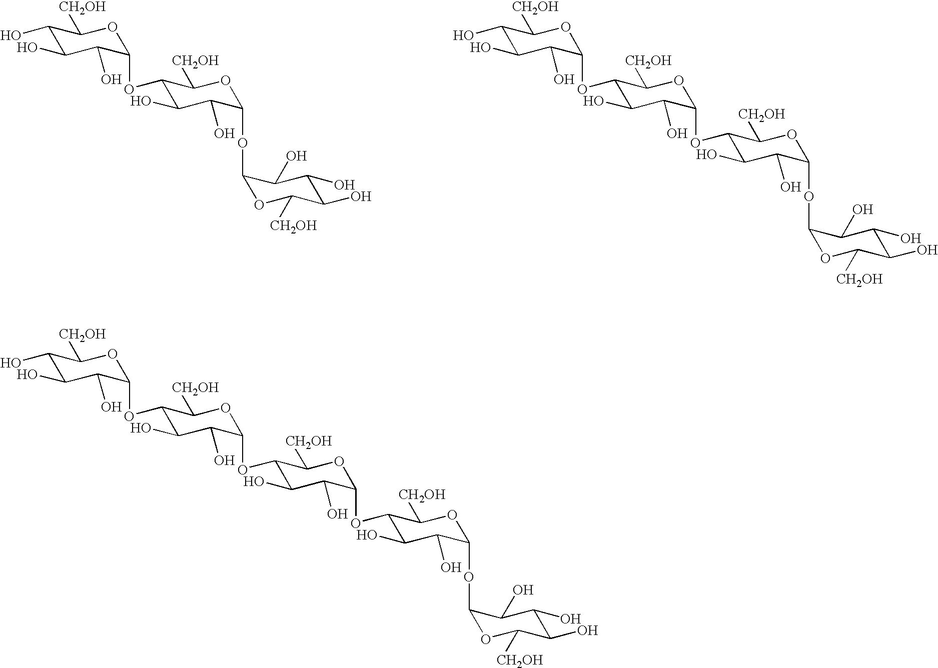

From the above results, it can be concluded that the enzyme of the present invention acts on maltotriose or a larger glucose polymers in which the glucose residues are α-1,4-linked, and transfers the first linkage from the reducing end into an α-1,α-1-linkage. Further, the enzyme of the present invention was found to hydrolyze the first linkage from the reducing end utilizing a H₂O molecule as the receptor to liberate a molecule of glucose, as is often observed in glycosyltransferases.

### EXAMPLE I-8

### Production of Glucosyltrehalose and Maltooligosyltrehalose from a Mixture of Maltooligosaccharides

Production of glucosyltrehalose and various maltooligosyltrehaloses was attempted by using 10 Units/ml of the purified enzyme obtained in Example I-2, and by using hydrolysate of a soluble starch product (manufactured by Nacalai tesque Co., special grade) with α-amylase as the substrate, wherein the soluble starch product had been hydrolyzed into oligosaccharides which did not exhibit the color of the iodo-starch reaction, by the α-amylase which was the A-0273 derived from Aspergillus oryzae manufactured by Sigma Co. The resultant reaction mixture was analyzed by an HPLC analysis method under the conditions below.

| | |
|---|---|
| Column: | BIORAD AMINEX HPX-42A (7.8 × 300 mm) |
| Solvent: | Water |
| Flow rate: | 0.6 ml/min. |
| Temperature: | 85° C. |
| Detector: | Refractive Index Detector |

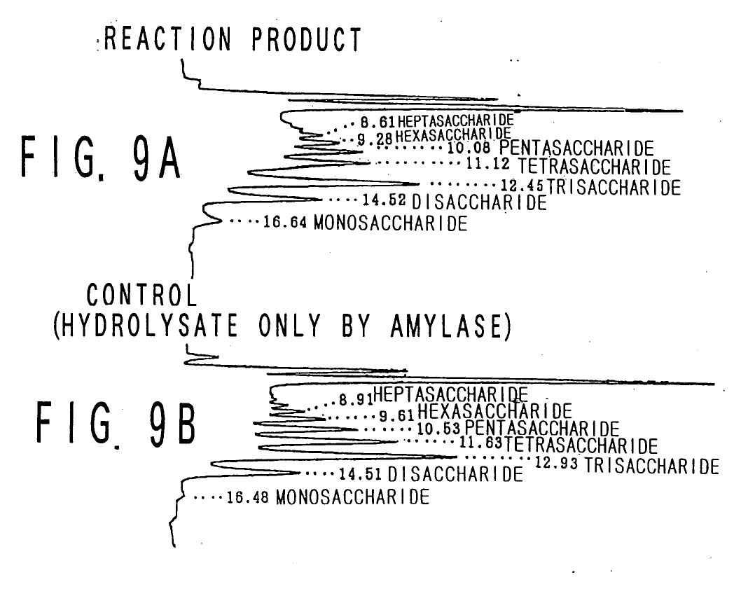

FIG. 9(A) is an HPLC analysis chart obtained herein. As a control, the HPLC chart of the case performed without the addition of the present transferase is shown in FIG. 9(B).

As a result, each of the oligosaccharides as the reaction products was found to have a retention time shorter than that of the control product which was produced using amylase only, wherein the shorter retention time is attributed to the α-1,α-1-transference of the reducing end of the oligosaccharides. Similar to Example I-7, the trisaccharide, the tetrasaccharide and the pentasaccharide were sampled and analyzed by ¹H-NMR and ¹³C-NMR. As a result, it was found that the glucose residue at the reducing end of each saccharide was α-1,α-1-linked, and those saccharides were recognized as glucosyltrehalose (α-D-maltosyl α-D-glucopyranoside), maltosyltrehalose (α-D-maltotriosyl α-D-glucopyranoside), and maltotriosyl-trehalose (α-D-maltotetraosyl α-D-glucopyranoside), respectively. The chemical formulae of these saccharides are as follows.

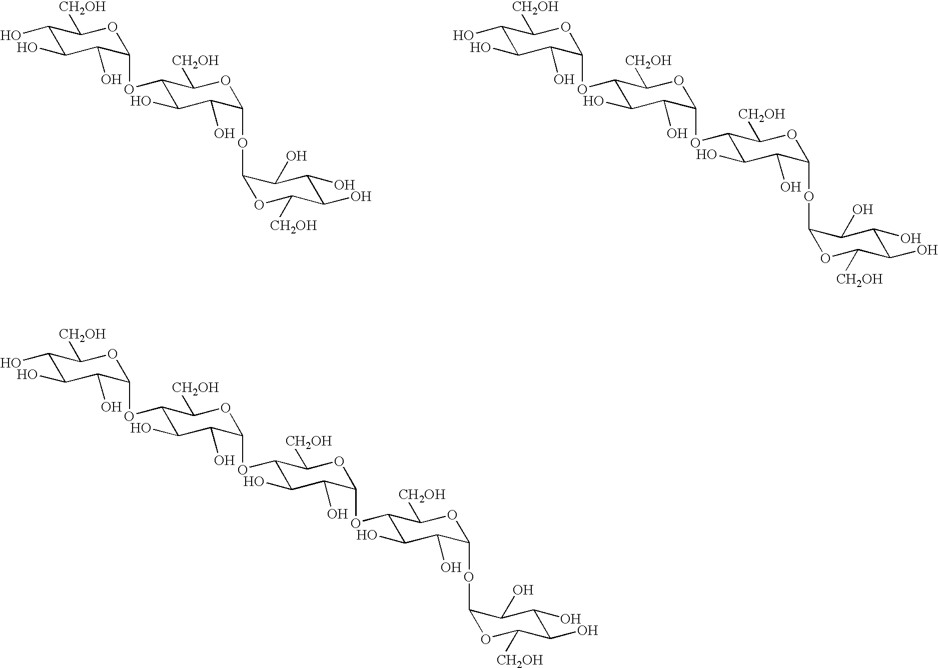

The reagents and materials described below, which were used in Examples II-1-II-14 (including Comparative Examples II-1 and II-2, and Referential Examples II-1-II-4), were obtained from the manufacturers described below, respectively.

α,α-trehalose: manufactured by Sigma Co.

Soluble starch: manufactured by Nacalai tesque Co., special grade

Pullulanase derived from Klebsiella pneumoniae: manufactured by Wako pure chemical Co., #165-15651

Pine-dex #1 and Pine-dex #3: manufactured by Matsutani Kagaku Co.

Maltose (G2): manufactured by Wako pure chemical Co.

Maltotriose (G3), Maltotetraose (G4), Maltopentaose (G5), Maltohexaose (G6), Maltoheptaose (G7), and Amylose DP-17: manufactured by Hayashibara Biochemical Co.

Amylopectin: manufactured by Nacalai tesque Co., special grade

Isomaltose: manufactured by Wako pure chemical Co.

Isomaltotriose: manufactured by Wako pure chemical Co.

Isomaltotetraose: manufactured by Seikagaku Kougyou Co.

Isomaltopentaose: manufactured by Seikagaku Kougyou Co.

Panose: manufactured by Tokyo Kasei Kougyou Co.

### EXAMPLE II-1

### Measurement of Trehaloseoligosaccharide-hydrolyzing Activity and Starch-liquefying Activity Possessed by Archaebacteria

The bacterial strains listed in Table 11 below were examined for enzymatic activity. The measurement was performed as follows: The cultivated cells of each bacterial strain were crushed by ultrasonic treatment and centrifuged; maltotriosyltrehalose as a substrate was added to the resultant supernatant, namely, a crude enzyme solution, so that the final concentration of maltotriosyltrehalose would be 10 mM; the mixture thus obtained was subjected to a reaction at 60° C. and pH 5.5 (50 mM sodium acetate buffer solution); the reaction was then stopped by heat-treatment at 100° C. for 5 min.; and the α,α-trehalose thus produced was analyzed by an HPLC method under the conditions below.

| | |
|---|---|
| Column: | TOSOH TSK-gel Amide-80 (4.6 × 250 mm) |
| Solvent: | 72.5% acetonitrile |
| Flow rate: | 1.0 ml/min. |
| Temperature: | Room temperature |
| Detector: | Refractive index detector |

The trehaloseoligosaccharide-hydrolyzing activity is expressed with such a unit as 1 Unit equals the activity of liberating 1 μmol of α,α-trehalose per hour from maltotriosyltrehalose. Incidentally, in Table 11, the activity is expressed in terms of units per one gram of bacterial cell. Here, maltotriosyltrehalose was prepared as follows: The purified transferase derived from the Sulfolobus solfataricus strain KM1 was added to a 10% maltopentaose solution containing 50 mM of acetic acid (pH 5.5) so that the concentration of the transferase would be 10 Units/ml; the mixture thus obtained was subjected to a reaction at 60° C. for 24 hours; and the resultant was subjected to the above TSK-gel Amide-80 HPLC column to obtain maltotriosyltrehalose. As to the activity of the purified transferase derived from the Sulfolobus solfataricus strain KM1, 1 Unit is defined as equalling the activity of producing 1 μmol of glucosyltrehalose per hour at 60° C. and pH 5.5 when maltotriose is used as the substrate.

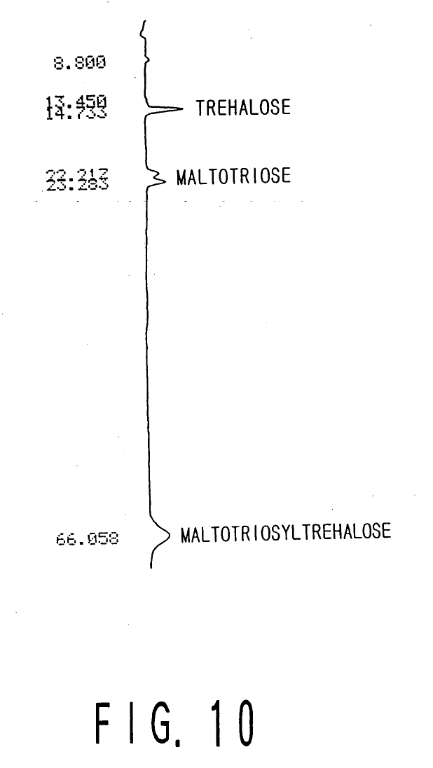

FIG. 10 is the HPLC chart obtained herein. As is recognized from the figure, a peak exhibiting the same retention time as that of α,α-trehalose without any anomer, and a peak exhibiting the same retention time as that of maltotriose appeared in the chart. Additionally, the product of the former peak was sampled by the TSK-gel Amide-80 HPLC column, and analyzed by ¹H-NMR and ¹³C-NMR. As a result, the product was confirmed to be α,α-trehalose.

Further, 2% soluble starch contained in a 100 mM sodium acetate buffer solution (pH 5.5) was subjected to a reaction with the above crude enzyme solution (the supernatant) at 60° C. by adding 0.5 ml of the supernatant to 0.5 ml of the starch solution. Time-course sampling was performed, and to each sample, twice volume of 1 N HCl was added for stopping the reaction. Subsequently, two-thirds volume of a 0.1% potassium iodide solution containing 0.01% of iodine was added, and further, 1.8-fold volume of water was added. Finally, absorptivity at 620 nm was measured, and the activity was estimated from the time-course change of the absorptivity.

The saccharides produced in the reaction were analyzed by an HPLC analysis method under the conditions shown below after the reaction was stopped by treatment at 100° C. for 5 min.

| | |
|---|---|
| Column: | BIORAD AMINEX HPX-42A (7.8 × 300 mm) |
| Solvent: | Water |
| Flow rate: | 0.6 ml/min. |
| Temperature: | 85° C. |
| Detector: | Refractive index detector |

As to starch-hydrolyzing activity, 1 Unit is defined as equalling the amount of the enzyme with which the absorptivity at 620 nm corresponding to the violet color of the starch-iodine complex decreases at a rate of 10% per 10 min. Incidentally, in Table 11, the activity was expressed in terms of units per one gram of bacterial cell.

TABLE 11

| | | Enzyme activity | |
|---|---|---|---|
| | | (uints/g-cell) | |
| | | Hydrolyzing | Hydrolyzing activity |
| | | activity | of trehalose |
| Strain | | of starch | oligosaccharide |
| Sulfolobus | ATCC 35091 | 13.3 | 118.0 |
| solfataricus | | | |
| | DSM 5354 | 13.3 | 116.8 |
| | DSM 5833 | 8.4 | 94.9 |
| | KM1 | 13.4 | 293.2 |
| Sulfolobus | ATCC 33909 | 12.5 | 161.8 |
| acidocaldarius | | | |
| Sulfolobus | DSM 5389 | 11.2 | 281.2 |
| shibatae | | | |

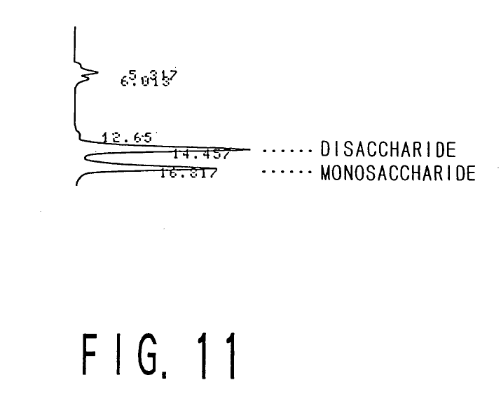

FIG. 11 shows the results of an analysis by AMINEX HPX-42A HPLC performed on the products by the reaction with the crude enzyme solution derived from the Sulfolobus solfataricus strain KM1.

From the above results, the cell extract of a bacterial strain belonging to the genus Sulfolobus was found to have an activity of hydrolyzing trehaloseoligosaccharides to liberate α,α-trehalose, and an activity of hydrolyzing starch to liberate principally monosaccharides or disaccharides.

### EXAMPLE II-2

### Purification of the Present Amylase Derived from the Sulfolobus solfataricus Strain KM1

The Sulfolobus solfataricus strain KM1 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 3.3 g/liter.

Two hundred grams of the bacterial cells obtained above were suspended in 400 ml of a 50 mM sodium acetate buffer solution (pH 5.5) containing 5 mM of EDTA, and subjected to ultrasonic treatment for bacteriolysis at 0° C. for 15 min. The resultant was then centrifuged to obtain a supernatant, and ammonium sulfate was added to the supernatant so as to be 60% saturation.

The precipitate obtained by centrifugation was dissolved in a 50 mM sodium acetate buffer solution (pH 5.5) containing 1 M of ammonium sulfate and 5 mM of EDTA, and subjected to hydrophobic chromatography using the TOSOH TSK-gel Phenyl-TOYOPEARL 650S column (volume: 800 ml) equilibrated with the same buffer solution as above. The column was then washed with the same buffer solution, and the objective amylase was eluted with 600 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM Tris-HCl buffer solution (pH 7.5).

Next, the resultant was subjected to ion-exchange chromatography using the TOSOH TSK-gel DEAE-TOYOPEARL 650S column (volume: 300 ml) equilibrated with the same buffer solution. The column was then washed with the same buffer solution, and the objective amylase was eluted with 900 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Subsequent to that, the desalted and concentrated solution thus obtained was subjected to gel filtration chromatography using the Pharmacia HiLoad 16/60 Superdex 200pg column, and the objective amylase was eluted with the same buffer solution. The fractions exhibiting the activity were concentrated using an ultrafiltration membrane (critical molecular weight: 13,000), and subsequently, washed and desalted with a 25 mM Bis-Tris-HCl buffer solution (pH 6.3).

Next, the desalted and concentrated solution thus obtained was subjected to a chromatofocusing using the Pharmacia Mono P HR/5/20 column equilibrated with the same buffer solution. The objective amylase was then eluted with 10% polybuffer 74 (manufactured by Pharmacia Co., and adjusted at pH 4.0 with HCl). The fractions exhibiting the activity were concentrated using an ultrafiltration membrane (critical molecular weight: 13,000), and subsequently, washed and desalted with a 10 mM sodium acetate buffer solution (pH 6.8).

Further, to this desalted and concentrated solution, a quarter volume of a sample buffer [62.5 mM Tris-HCl buffer solution (pH 6.8), 10% glycerol, 2% SDS, and 0.0125% Bromophenolblue] was added, and subjected to 10% SDS-Polyacrylamide gel electrophoresis (SDS-PAGE) (apparatus: BIO-RAD Prep Cell Model 491) to elute the objective amylase. The fractions exhibiting the activity were separated and concentrated using an ultrafiltration membrane (critical molecular weight: 13,000), and subsequently, washed and desalted with a 10 mM sodium acetate buffer solution (pH 5.5).

Finally, Native polyacrylamide gel electrophoresis, SDS-polyacrylamide gel electrophoresis and isoelectric focusing were performed to obtain the purified enzyme which appeared as single band.

Incidentally, for the activity measurement, in this purification procedure, maltotriosyltrehalose was used as the substrate, and the same manner as in the TSK-gel Amide-80 HPLC analysis method shown in Example II-1 was employed.

Total enzyme activity, total protein and specific activity at each of the purification steps are shown in Table 12 below.

TABLE 12

| | Total | | | | |
|---|---|---|---|---|---|
| | enzyme | Total | Specific | | |
| | activity | protein | activity | | Purity |
| Purified fraction | (units) | (mg) | (units/mg) | Yield (%) | (fold) |
| 60% saturated | 58640 | 17000 | 3.45 | 100 | 1 |
| (NH₄)₂SO₄ | | | | | |
| precipitation | | | | | |
| Phenyl | 52251 | 1311 | 39.9 | 89 | 12 |
| DEAE | 49284 | 195 | 253 | 84 | 73 |
| Gel-permeation | 2197 | 26.7 | 82.2 | 3.7 | 24 |
| Mono P | 1048 | 0.40 | 2640 | 1.8 | 765 |
| SDS-PAGE | 401 | 0.08 | 5053 | 0.7 | 1465 |

### EXAMPLE II-3

### Purification of the Present Amylase Derived from the Sulfolobus solfataricus Strain DSM 5833

The Sulfolobus solfataricus strain DSM 5833 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 1.2 g/liter.

Twenty five grams of the bacterial cells obtained above were suspended in 50 ml of a 50 mM sodium acetate buffer solution (pH 5.5) containing 5 mM of EDTA, and subjected to ultrasonic treatment for bacteriolysis at 0° C. for 15 min. The resultant was then centrifuged to obtain a supernatant.

To this supernatant, ammonium sulfate was added so as to be 1 M. The resultant was then subjected to hydrophobic chromatography using TOSOH TSK-gel Phenyl-TOYOPEARL 650S column (volume: 100 ml) equilibrated with a 50 mM sodium acetate buffer solution (pH 5.5) containing 1 M of sodium sulfate and 5 mM of EDTA. The column was then washed with the same buffer solution, and the objective amylase was eluted with 300 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM Tris-HCl buffer solution (pH 7.5).

Next, the resultant was subjected to ion-exchange chromatography using the TOSOH TSK-gel DEAE-TOYOPEARL 650S column (volume: 100 ml) equilibrated with the same buffer solution. The column was then washed with the same buffer solution, and the objective amylase was eluted with 300 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Subsequent to that, the desalted and concentrated solution thus obtained was subjected to gel filtration chromatography using the Pharmacia HiLoad 16/60 Superdex 200pg column, and the objective amylase was eluted with the same buffer solution. The fractions exhibiting the activity were concentrated using an ultrafiltration membrane (critical molecular weight: 13,000), and subsequently, washed and desalted with a 25 mM Bis-Tris-iminodiacetic acid buffer solution (pH 7.1).

Next, the desalted and concentrated solution thus obtained was subjected to a chromatofocusing using the Pharmacia Mono P HR5/20 column equilibrated with the same buffer solution. The objective amylase was then eluted with 10% Polybuffer 74 (manufactured by Pharmacia, and adjusted at pH 4.0 with iminodiacetic acid). The fractions exhibiting the activity were concentrated using an ultrafiltration membrane (critical molecular weight: 13,000), and subsequently, washed and desalted with a 25 mM bis-Tris-iminodiacetic acid buffer solution (pH 7.1).

Further, the desalted and concentrated solution thus obtained was subjected to a chromatofocusing using the Pharmacia Mono P HR5/20 column equilibrated with the same buffer solution. The objective amylase was then eluted with 10% Polybuffer 74 (manufactured by Pharmacia, and adjusted at pH 4.0 with iminodiacetic acid). The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Moreover, the desalted and concentrated solution thus obtained was subjected to gel filtration chromatography using the TSK-gel G3000SW HPLC column, and the objective amylase was then eluted with the same buffer solution. The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Finally, Native Polyacrylamide gel electrophoresis, SDS-Polyacrylamide gel electrophoresis and isoelectric focusing were performed to obtain the purified enzyme which appeared as single band.

Incidentally, for the activity measurement, in this purification procedure, maltotriosyltrehalose was used as the substrate, and the same manner as in the TSK-gel Amide-80 HPLC analysis method shown in Example II-1 was employed.

Total enzyme activity, total protein and specific activity at each of the purification steps are shown in Table 13 below.

TABLE 13

| | Total enzyme | Total | Specific | | |
|---|---|---|---|---|---|
| | activity | protein | activity | Yield | Purity |
| Purified fraction | (units) | (mg) | (units/mg) | (%) | (fold) |
| Crude extract | 3345 | 1394 | 2.40 | 100 | 1 |
| Phenyl | 2112 | 266 | 7.9 | 63 | 3.3 |
| DEAE | 1365 | 129 | 10.6 | 41 | 4.4 |
| Gel-permeation | 651 | 7.8 | 83.5 | 19 | 35 |
| Mono P | 467 | 0.76 | 612 | 14 | 255 |
| Mono P | 156 | 0.12 | 1301 | 4.7 | 542 |
| rechromatography | | | | | |
| Gel-permeation | 98 | 0.01 | 13652 | 2.9 | 5687 |
| rechromatography | | | | | |

### EXAMPLE II-4

### Purification of the Present Amylase Derived from the Sulfolobus acidocaldarius strain ATCC 33909

The Sulfolobus acidocaldarius strain ATCC 33909 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 2.7 g/liter.

Twenty five grams of the bacterial cells obtained above were suspended in 50 ml of a 50 mM sodium acetate buffer solution (pH 5.5) containing 5 mM of EDTA, and subjected to ultrasonic treatment for bacteriolysis at 0° C. for 15 min. The resultant was then centrifuged to obtain a supernatant.

To this supernatant, ammonium sulfate was added so as to be 1 M. The resultant was then subjected to hydrophobic chromatography using TOSOH TSK-gel Phenyl-TOYOPEARL 650S column (volume: 100 ml) equilibrated with a 50 mM sodium acetate buffer solution (pH 5.5) containing 1 M of sodium sulfate and 5 mM of EDTA. The column was then washed with the same buffer solution, and the objective amylase was eluted with 300 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 10 mM Tris-HCl buffer solution (pH 7.5).

Next, the resultant was subjected to ion-exchange chromatography using the TOSOH TSK-gel DEAE-TOYOPEARL 650S column (volume: 100 ml) equilibrated with-the same buffer solution. The column was then washed with the same buffer solution, and the objective amylase was eluted with 300 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Subsequent to that, the desalted and concentrated solution thus obtained was subjected to gel filtration chromatography using the Pharmacia HiLoad 16/60 Superdex 200pg column, and the objective amylase was eluted with the same buffer solution. The fractions exhibiting the activity were concentrated using an ultrafiltration membrane (critical molecular weight: 13,000), and subsequently, washed and desalted with a 50 mM sodium acetate buffer solution (pH 5.5).

Next, ammonium sulfate was dissolved in the desalted and concentrated solution so that the concentration of ammonium sulfate would be 1 M. The resultant was then subjected to hydrophobic chromatography using TOSOH TSK-gel Phenyl-5PW HPLC column equilibrated with the same buffer solution. The column was then washed with the same buffer solution, and the objective amylase was eluted with 30 ml of ammonium sulfate solution at a linear concentration gradient from 1 M to 0 M. The fractions exhibiting the activity were concentrated using an ultrafiltration membrane (critical molecular weight: 13,000), and subsequently, washed and desalted with a 25 mM bis-Tris-iminodiacetic acid buffer solution (pH 7.1).

Further, the desalted and concentrated solution thus obtained was subjected to a chromatofocusing using the Pharmacia Mono P HR5/20 column equilibrated with the same buffer solution. The objective amylase was then eluted with 10% Polybuffer 74 (manufactured by Pharmacia, and adjusted at pH 4.0 with iminodiacetic acid). The fractions exhibiting the activity were concentrated using an ultrafiltration membrane (critical molecular weight: 13,000), and subsequently, washed and desalted with a 50 mM sodium acetate buffer solution (pH 5.5) containing 5 mM of EDTA.

Finally, Native Polyacrylamide gel electrophoresis, SDS-Polyacrylamide gel electrophoresis and isoelectric focusing were performed to obtain the purified enzyme which appeared as single band.

Incidentally, for the activity measurement, in this purification procedure, maltotriosyltrehalose was used as the substrate, and the same manner as in the TSK-gel Amide-80 HPLC analysis method shown in Example II-1 was employed.

Total enzyme activity, total protein and specific activity at each of the purification steps are shown in Table 14 below.

TABLE 14

| | Total enzyme | Total | Specific | | |
|---|---|---|---|---|---|
| | activity | protein | activity | Yield | Purity |
| Purified fraction | (units) | (mg) | (units/mg) | (%) | (fold) |
| Crude extract | 4534 | 760 | 5.97 | 100 | 1 |
| Phenyl | 2428 | 88.0 | 27.6 | 54 | 4.6 |
| DEAE | 927 | 9.20 | 101 | 20 | 17 |
| Gel-permeation | 600 | 1.10 | 546 | 13 | 92 |
| Phenyl | 392 | 0.16 | 2449 | 9.1 | 411 |
| rechromatography | | | | | |
| Mono P | 120 | 0.04 | 3195 | 2.6 | 558 |

### EXAMPLE II-5

### Examination of the Present Amylase for Various Characteristics

The purified enzyme obtained in Example II-2 was examined for enzymatic characteristics.

(1) Molecular Weight

The molecular weight was measured by SDS-polyacrylamide gel electrophoresis (gel concentration; 6%). Marker proteins having molecular weights of 200,000, 116,300, 97,400, 66,300, 55,400, 36,500, 31,000, 21,500 and 14,400, respectively, were used.

As a result, the molecular weight of the amylase was estimated at 61,000.

(2) Isoelectric Point

The isoelectric point was found to be pH 4.8 by agarose gel isoelectric focusing.

(3) Stability

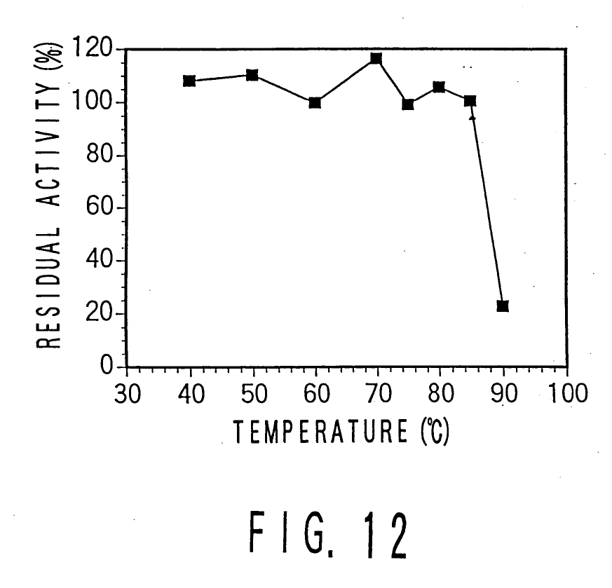

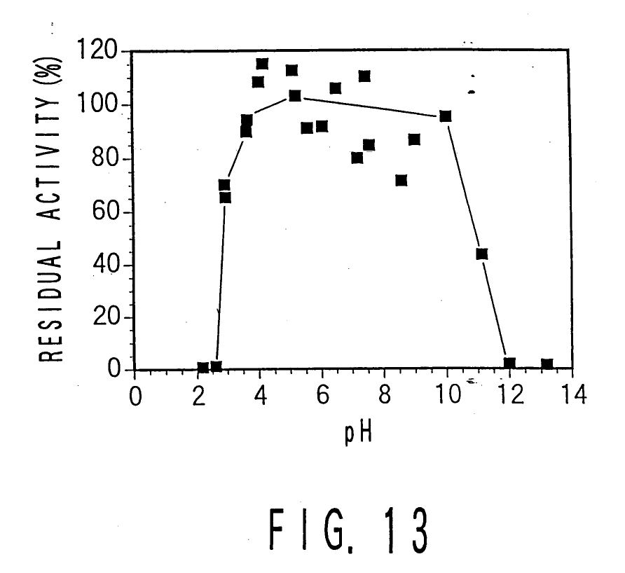

The stability changes of the obtained enzyme according to temperature and pH value are shown in FIGS. 12 and 13, respectively. The measurement of enzymatic activity was carried out according to the measurement method in Example II-1 using maltotriosyltrehalose, and a glycine-HCl buffer solution was used in a pH range of 3-5, and similarly, a sodium, acetate buffer solution in a pH range of 4-6, a sodium phosphate buffer solution in a pH range of 5-8, a Tris-HCl buffer solution in a pH range of 8-9, a sodium bicarbonate buffer solution in a pH range of 9-10, and a KCl-NaOH buffer solution in a pH range of 11-13.5, respectively, were also used.

The present enzyme was stable throughout the treatment at 85° C. for 6 hours, and also, was stable throughout the treatment at pH 3.5-10.0 and room temperature for 6 hours.

(4) Reactivity

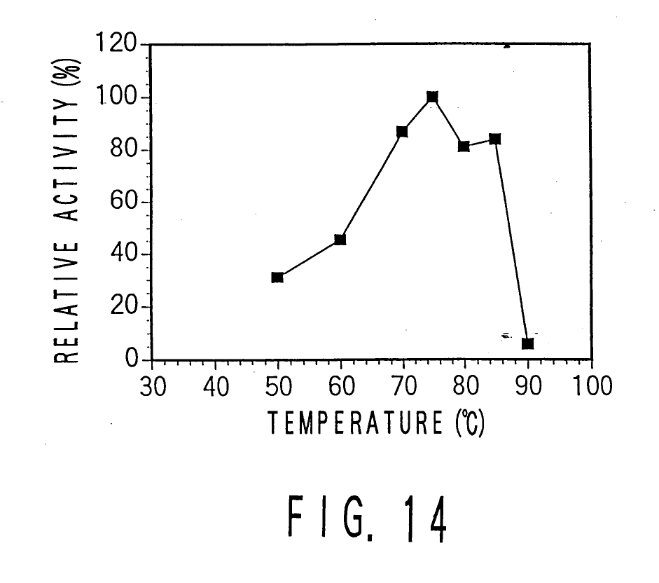

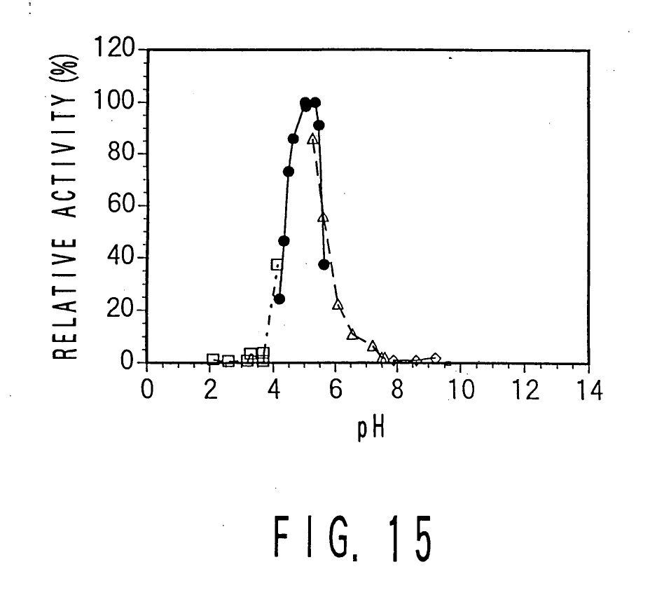

As to the obtained enzyme, reactivity at various temperatures and reactivity at various pH are shown in FIGS. 14 and 15, respectively. The measurement of enzymatic activity was carried out according to the measurement method in Example II-1 using maltotriosyltrehalose, and a sodium citrate buffer solution was used in a pH range of 2-4 (□), and similarly, a sodium acetate buffer solution in a pH range of 4-5.5 (●), a sodium phosphate buffer solution in a pH range of 5-7.5 (Δ), and a Tris-HCl buffer solution in a pH range of 8-9 (⋄), respectively, were also used.

The optimum reaction temperature of the present enzyme is within 70-85° C., approximately, and the optimum reaction pH of the present enzyme is within 4.5-5.5, approximately.

(5) Influence of Various Activators and Inhibitors

The influence of each substance listed in Table 15, such as an activating effect or inhibitory effect, was evaluated using similar activity-measuring method to that in Example II-1. Specifically, the listed substances were individually added together with the substrate to the same reaction system as that in the method for measuring maltotriosyltrehalose-hydrolyzing activity employed in Example II-1. As a result, copper ion and sodium dodecyl sulfate (SDS) were found to have inhibitory effects. As to the inhibitory effect by SDS, however, the enzymatic activity revived after SDS was removed by dialysis, ultrafiltration or the like. Though many glucide-relating enzymes have been found to be activated with calcium ion, the present enzyme would not be activated with calcium ion.

TABLE 15

| | Concentration | Residual activity |
|---|---|---|
| Activator/Inhibitor | (mM) | (%) |
| Control (not added) | | 100.0 |
| CaCl₂ | 5 | 97.1 |
| MgCl₂ | 5 | 93.5 |
| MnCl₂ | 5 | 101.8 |
| CuSO₄ | 5 | 0 |
| CoCl₂ | 5 | 97.1 |
| FeSO₄ | 5 | 73.5 |
| FeCl₃ | 5 | 38.0 |
| AgNO₃ | 5 | 105.7 |
| EDTA | 5 | 106.3 |
| 2-Mercaptoethanol | 5 | 141.7 |
| Dithiothreitol | 5 | 116.2 |
| SDS | 5 | 0 |
| Glucose | 0.5 | 109.4 |
| α,α-Trehalose | 0.5 | 98.2 |
| Maltotetraose | 0.5 | 108.5 |
| Malatopentaose | 0.5 | 105.8 |
| Maltohexaose | 0.5 | 123.8 |
| Maltoheptaose | 0.5 | 129.2 |

(6) Substrate Specificity

The hydrolyzing properties were analyzed by allowing 25.0 Units/ml (in terms of the enzymatic activity when maltotriosyltrehalose is used as the substrate) of the present purified enzyme to act on the various 10 mM substrates (except amylopectin and soluble starch were used as 2.8% solutions) listed in Table. 16 below, and the hydrolyzed products were also analyzed. The analysis was performed by TSK-gel Amide-80 HPLC described in Example II-1, wherein the index was the activity of producing both monosaccharide and disaccharide when the substrate was each of the various maltooligosaccharides, Amylose DP-17, amylopectin, soluble starch, various isomaltooligosaccharides, and panose; the activity of producing α,α-trehalose when the substrate was each of the various trehaloseoligosaccharides, and α-1,α-1-transferred isomer of Amylose DP-17 (the oligosaccharide derived from Amylose DP-17 by transferring the linkage between the first and second glucose residues from the reducing end into an α-1,α-1 linkage); and the activity of producing glucose when the substrate was maltose or α,α-trehalose.

Incidentally, each enzymatic.activity in Table 16 is expressed with such a unit as 1 Unit equals the activity of liberating 1 μmol of each of the monosaccharide and disaccharide per hour.

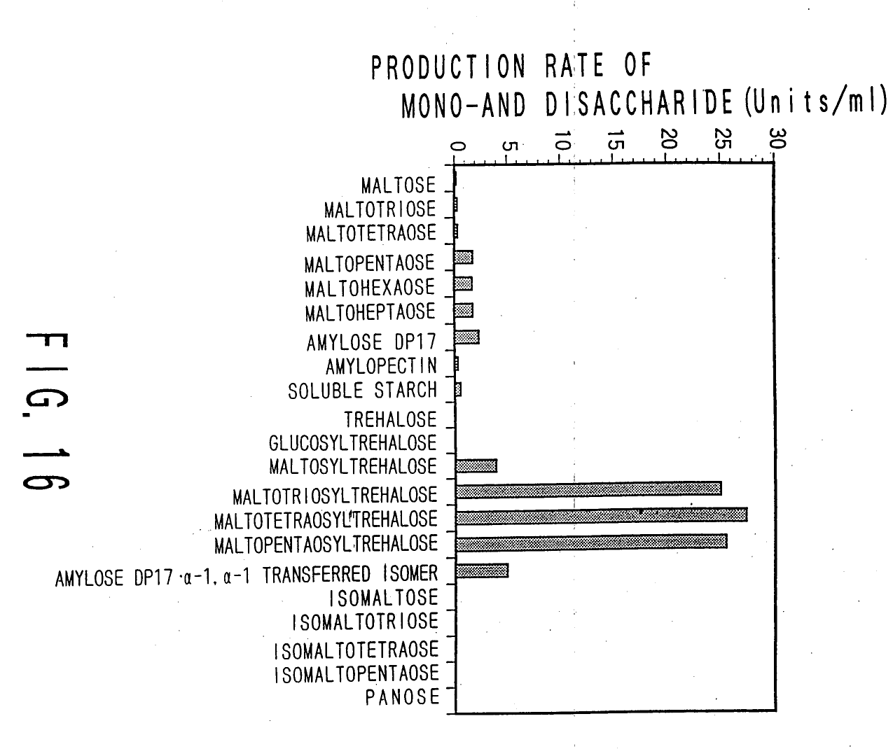

The results are as shown in Table 16 below and in FIGS. 16-19.

TABLE 16

| | | Production rate |
|---|---|---|
| | | of mono- and |
| | Liberated | disaccharides |
| Substrate | oligosaccharide | (units/ml) |
| Maltose (G2) | Glucose | 0.19 |
| Maltotriose (G3) | Glucose + G2 | 0.30 |
| Maltotetraose (G4) | Glucose + G2 + G3 | 0.31 |
| Maltopentaose (G5) | Glucose + G2 + G3 + G4 | 1.79 |
| Maltohexaose (G6) | Glucose + G2 + G4 + G5 | 1.74 |
| Maltoheptaose (G7) | Glucose + G2 + G5 + G6 | 1.80 |
| Amylose DP-17 | Glucose + G2 | 2.35 |
| Amylopectin | Glucose + G2 | 0.33 |
| Soluble starch | Glucose + G2 | 0.55 |
| α, α-Trehalose | not decomposed | 0 |
| Glucosyltrehalose | Glucose + Trehalose | 0.04 |
| Maltosyltrehalose | G2 + Trehalose | 3.93 |
| Maltotriosyltrehalose | G3 + Trehalose | 25.0 |
| Maltotetraosyltrehalose | G4 + Trehalose | 27.3 |
| Maltopentaosyltrehalose | G5 + Trehalose | 25.5 |
| Amylose DP-17, α-1, | Trehalose | 4.98 |
| α-1-transferred isomer | | |
| Isomaltose | not decomposed | 0 |
| Isomaltotriose | not decomposed | 0 |
| Isomaltotetraose | not decomposed | 0 |
| Isomaltopentaose | not decomposed | 0 |
| Panose | not decomposed | 0 |

Notes: Each of glucosyltrehalose, maltosyltrehalose, maltotetraosyltrehalose, maltopentaosyltrehalose, and α-1,α-1-transferred isomer of Amylose DP-17 was prepared according to the method for preparing maltotriosyltrehalose in Example II-1.

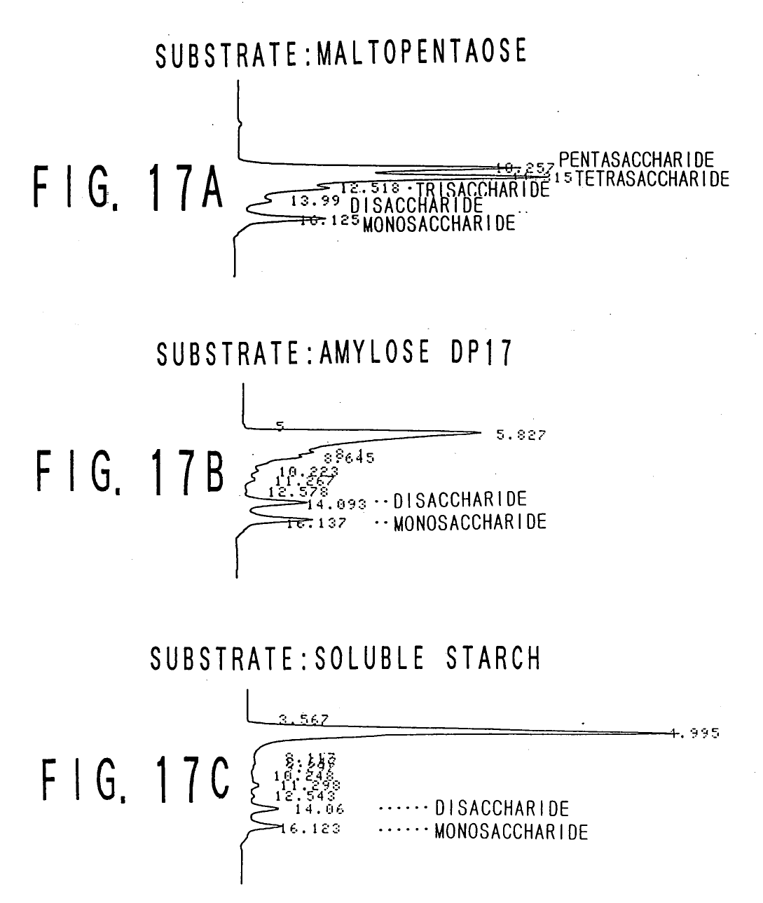

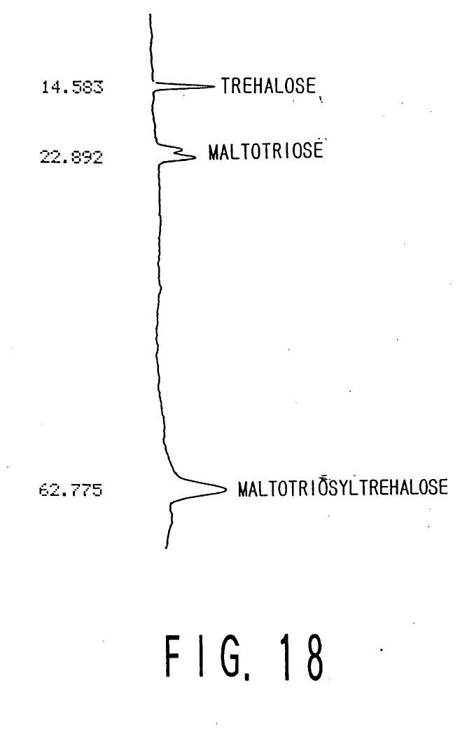

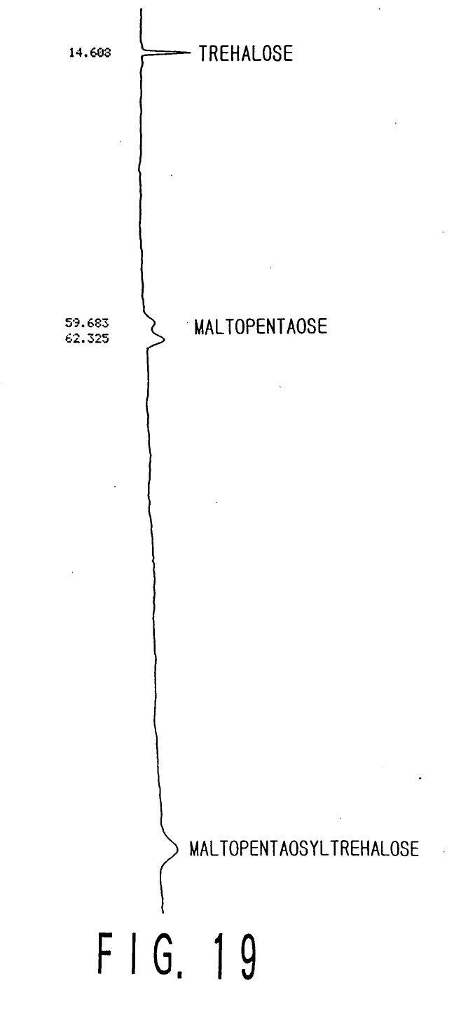

The results of the analyses by AMINEX HPX-42A HPLC performed on reaction products from maltopentaose, Amylose DP-17 and soluble starch are shown in A, B and C of FIG. 17, respectively. Further, the results of the analyses by TSK-gel Amide-80 HPLC performed on reaction products from maltotriosyltrehalose and maltopentaosyltrehalose are shown in FIGS. 18 and 19, respectively.

From the results, the present purified enzyme was confirmed to markedly effectively act on a trehaloseoligo-saccharide, of which the glucose residue at the reducing end side is α-1,α-1-linked, such as maltotoriosyltrehalose, to liberate α,α-trehalose and a corresponding maltooligosac-charide which has a polymerization degree reduced by two. Further, the present purified enzyme was confirmed to liberate principally glucose or maltose from maltose (G2)-maltoheptaose (G7), amylose, and soluble starch. The present purified enzyme, however, did not act on α,α-trehalose, which has an α-1,α-1 linkage; isomaltose, isomaltotriose, isomaltotetraose and isomaltopentaose, of which all the sugar units are α-1,6-linked; and panose, of which the second linkage from the reducing end is α-1,6.

(7) Endotype Amylase Activity

Two hundred Units/ml (in terms of the enzymatic activity when maltotriosyltrehalose is used as the substrate) of the present purified enzyme was allowed to act on soluble starch, and the time-lapse changes in the coloring degree by the iodo-starch reaction, and the starch-hydrolyzing rate estimated from the produced amounts of monosaccharide and disaccharide were analyzed using the method for measuring starch-hydrolyzing activity described in Example II-1, and the AMINEX HPX-42A HPLC analyzing method.

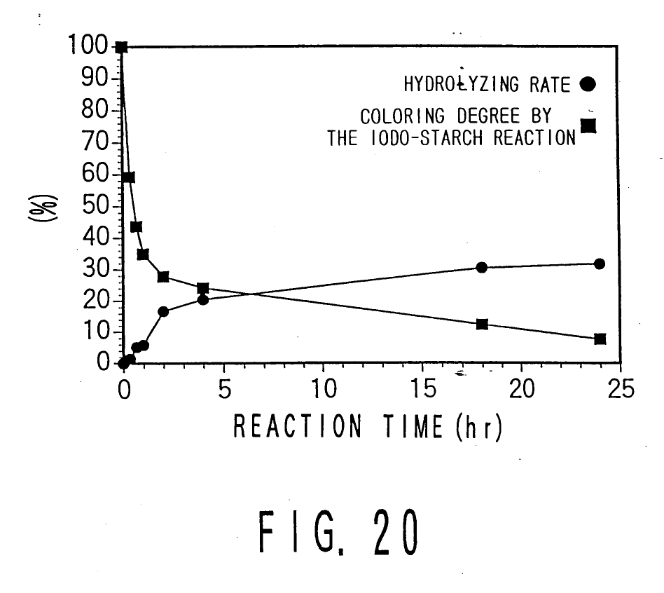

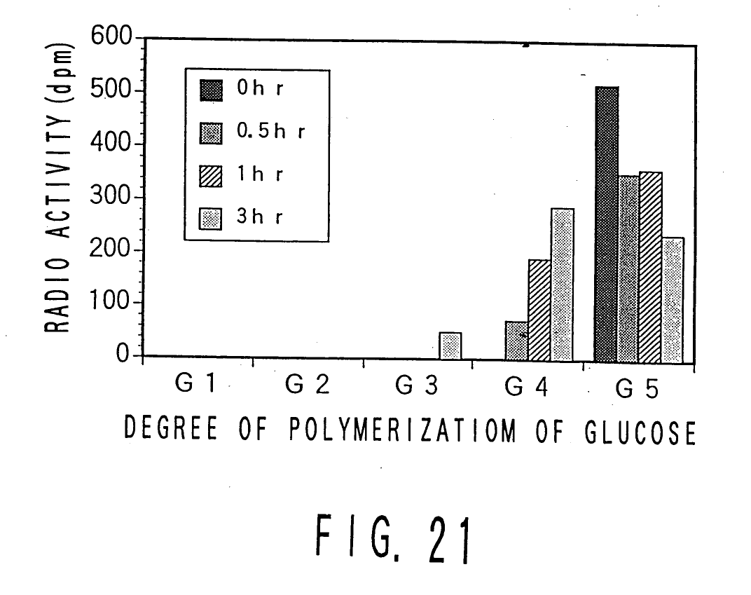

As shown in FIG. 20, the hydrolyzing rate of the present purified enzyme at the point where the coloring degree by the iodo-starch reaction decreased to 50% was as low as 3.7%. Accordingly, the present purified enzyme was confirmed to have a property of an endotype amylase.

(8) Investigation of the Action Mechanism

Uridinediphosphoglucose [glucose-6-³H] and maltotetraose were put into a reaction with glycogen synthase (derived from rabbit skeletal muscle, G-2259 manufactured by Sigma Co.) to synthesize maltopentaose, of which the glucose residue of the non-reducing end was radiolabeled with ³H, and the maltopentaose was isolated and purified. To 10 mM of this maltopentaose radiolabeled with ³H as a substrate, 10 Units/ml (in terms of the enzymatic activity when maltotriose is used as the substrate) of the purified transferase derived from the Sulfolobus solfataricus strain KM1 was added and put into a reaction at 60° C. for 3 hours. Maltotriosyltrehalose, of which the glucose residue of the non-reducing end was radiolabeled with ³H, was synthesized thereby, and the product was isolated and purified. [Incidentally, it was confirmed by the following procedure that the glucose residue of the non-reducing end had been radiolabeled: The above product was completely decomposed into glucose and α,α-trehalose by glucoamylase (derived from Rhizopus, manufactured by Seikagaku Kou.gyou Co.); the resultants were sampled by thin-layer chromatography, and their radioactivities were measured by a liquid scintillation counter; as a result, radioactivity was not observed in the α,α-trehalose fraction but in the glucose fraction.]

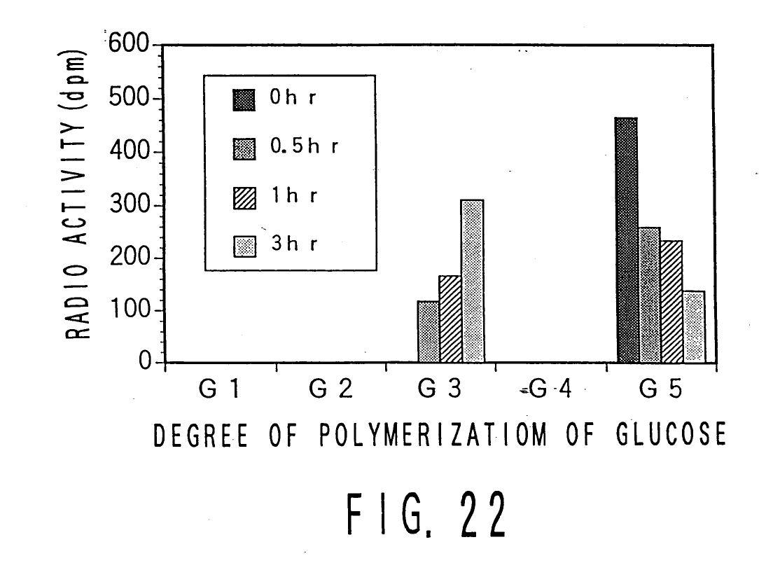

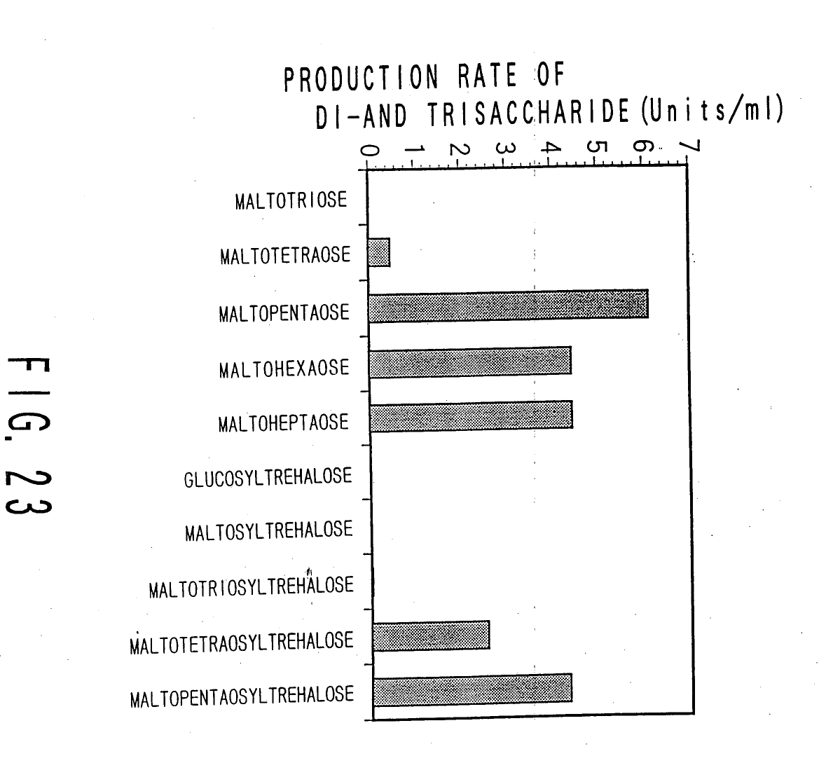

The above-prepared maltopentaose and maltotriosyltrehalose, of which the glucose residues of the non-reducing ends were radiolabeled with ³H, were used as substrates, and were put into reactions with 50 Units/ml and 5 Units/ml of purified enzyme obtained. in Example II-2, respectively. Sampling was performed before the reaction; and 0.5, 1 and 3 hours after the start of the reaction performed at 60° C. The reaction products were subjected to development by thin-layer chromatography (Kieselgel 60 manufactured by Merck Co.; solvent: butanol/ethanol/water=5/5/3). Each spot thus obtained and corresponding to each saccharide was collected, and its radiation was measured with a liquid scintillation counter. The results are shown in FIGS. 21 and 22, respectively.

As is obvious from FIGS. 21 and 22, when maltopentaose was used as a substrate, radioactivity was not detected in the fractions of the hydrolysates, i.e. glucose and maltose, but in the fractions of maltotetraose and maltotriose. On the other hand, when maltotriosyltrehalose was used as a substrate, radioactivity was not detected in the fraction of the hydrolysate, i.e. α,α-trehalose, but in the fraction of maltotriose.

Consequently, as to the action mechanism, the present purified enzyme was found to have an amylase activity of. the endotype function, and in addition, an activity of principally producing monosaccharide and disaccharide from the reducing end side.

Additionally, each of the purified enzymes obtained in Examples II-3 and II-4, i.e. derived from the Sulfolobus solfataricus strain DSM 5833 and the Sulfolobus acidocaldarius strain ATCC 33909, respectively, was also examined for the enzymatic characteristics in a similar manner. The results are shown in Table 2 above.

### Comparative Example II-1

### Properties of Pancreatic α-Amylase in Hydrolyzing Various Oligosaccharides, and Analysis of the Hydrolysates

Swine pancreatic α-amylase is known to hydrolyze maltooligosaccharide from the reducing end by two or three sugar units [“Denpun-Kanren Toushitsu Kouso Jikken-hou” (“Experimental methods in enzymes for starch and relating saccharides”), p 135, written by Michinori Nakamura and Keiji Kainuma, published by Gakkai-Shuppan-Sentah]. Upon this, a swine pancreatic α-amylase (manufactured by Sigma Co., A-6255) was analyzed the hydrolyzing properties and the hydrolysates as a comparative example for the novel amylase of the present invention. Specifically, 1 Unit/ml of the swine pancreatic α-amylase was allowed to act on 10 mM of each substrate listed in below-described Table 17 at pH 6.9 and 20° C., wherein 1 Unit is defined as equalling the amount of the enzyme with which 1 mg per 3 min. of a reducing saccharide corresponding to maltose is produced at pH 6.9 and 20° C. from starch assigned for the substrate. The activity of producing disaccharide and trisaccharide was employed as the index of the enzymatic activity, and the analysis was performed by the TSK-gel Amide-80 HPLC analyzing method described in Example II-1.

Incidentally, the enzymatic activity values in Table 17 were expressed with such a unit as 1 Unit equals the activity of liberating 1 μmol of each oligosaccharide per hour.

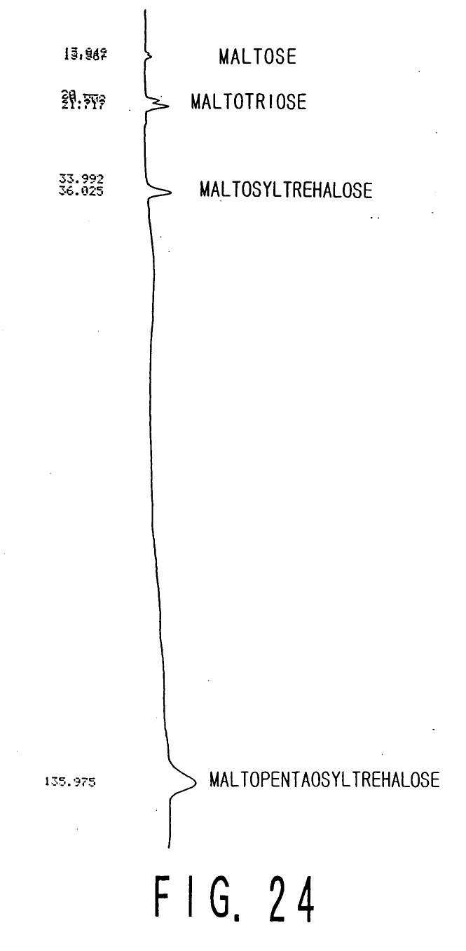

The results are shown in Table 17 below and in FIGS. 23 and 24.

TABLE 17

| | | Production rate |
|---|---|---|
| | | of di- and |
| | Liberated | trisaccharides |
| Substrate | oligosaccharide | (units/ml) |
| Maltotriose (G3) | not decomposed | 0 |
| Maltotetraose (G4) | Glucose + G2 + G3 | 0.49 |
| Maltopentaose (G5) | G2 + G3 | 6.12 |
| Maltohexaose (G6) | G2 + G3 + G4 | 4.44 |
| Maltoheptaose (G7) | G2 + G3 + G4 + G5 | 4.45 |
| Glucosyltrehalose | not decomposed | 0 |
| Maltosyltrehalose | not decomposed | 0 |
| Maltotriosyltrehalose | G2 + Glucosyltrehalose | 0.03 |
| Maltotetraosyltrehalose | G3 + Glucosyltrehalose | 2.57 |
| Maltopentaosyltrehalose | G3 + Maltosyltrehalose | 4.36 |

Notes: Each of glucosyltrehalose, maltosyltrehalose, maltotetraosyltrehalose, and maltopentaosyltrehalose was prepared according to the method for preparing maltotriosyltrehalose in Example II-1.

The results of analyses by TSK-gel Amide-80 HPLC performed on reaction products from maltopentaosyltrehalose are shown in FIG. 24.

From the results, the pancreatic amylase was confirmed to produce, from each of maltotetraose (G4)-maltoheptaose (G7), maltose or maltotriose, and a corresponding maltooligosaccharide which had a polymerization degree reduced by two or three; but not to liberate α,α-trehalose from trehaloseoligosaccharides such as glucosyltrehalose and maltooligosyltrehalose, of which the glucose-residue at the reducing end side is α-1,α-1-linked; and in addition, to have small reactivity to such trehaloseoligosaccharides.

### EXAMPLE II-6

### Production of α,α-Trehalose from Soluble Starch and Various Starch Hydrolysates

Production of α,α-trehalose utilizing the synergism between enzymes was attempted as follows:

The enzymes used were 150 Units/ml of the present purified enzyme obtained in Example II-2, and 10 Units/ml of the purified transferase derived from the Sulfolobus solfataricus strain KM1;

substrates were a soluble starch (manufactured by Nacalai tesque Co., special grade), as a starch hydrolysate, a soluble starch which had been subjected to hydrolysis of the α-1,6 linkages beforehand under the conditions of 40° C. for 1 hour with 25 Units/ml of pullulanase (manufactured by Wako pure. chemical Co.) derived from Klebsiella pneumoniae, as another starch hydrolysate, a soluble starch which had been subjected to partial hydrolysis beforehand under the conditions of 30° C. for 2.5 hours with 12.5 Units/ml of α-amylase (manufactured by Boehringer Mannheim Co.) derived from Bacillus amylolichefaciens, Pine-dex #1 and Pine-dex #3 (both manufactured by Matsutani Kagaku Co.), each maltooligosaccharide of G3-G7 (manufactured by Hayashibara Biochemical Co.), and Amylose DP-17 (manufactured by Hayashibara Biochemical Co.);

the final concentration of each substrate was 10%; and

each reaction was performed under the conditions of 60° C. at pH 5.5 for 100 hours, approximately.

Each reaction mixture was analyzed by the AMINEX HPX-42A HPLC method described in Example II-1, according to the case in which soluble starch was used as the substrate.

After the non-reacted substrate was hydrolyzed with glucoamylase, the yield of α,α-trehalose was analyzed by the TSK-gel Amide-80 HPLC analyzing method described in Example II-1.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotriose per minute at pH 6.0 and 30° C. from pullulan assigned for the substrate.

The results are shown in Table 18 below.

TABLE 18

| | Yield of |
|---|---|
| Substrate | α,α-trehalose (%) |
| Soluble starch | 37.0 |
| Pullulanase-treated starch | 62.1 |
| Amylase-treated starch | 42.2 |
| Pinedex #1 | 49.9 |
| Pinedex #3 | 40.4 |
| Maltotriose (G3) | 36.4 |
| Maltotetraose (G4) | 47.8 |
| Maltopentaose (G5) | 60.0 |
| Maltohexaose (G6) | 61.8 |
| Maltoheptaose (G7) | 67.1 |
| Amylose DP-17 | 83.5 |

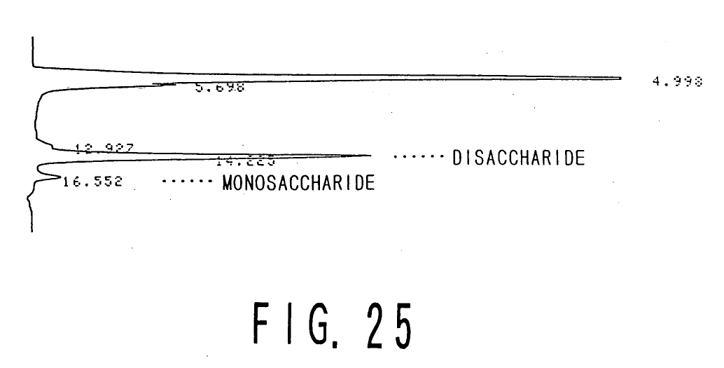

The results of the analysis by AMINEX HPX-42A HPLC performed on the reaction product from the soluble starch are shown in FIG. 25.

Specifically, when soluble starch was used as the substrate, α,α-trehalose was produced in a yield of 37.0%. As to the various starch hydrolysates, the yield was 62.1% when soluble starch which had been subjected to hydrolysis of the α-1,4 linkages was used as the substrate. Further, in the various maltooligosaccharides and Amylose DP-17, in which all of the linkages are α-1,4 linkages, the yields were 36.4-67.1%, and 83.5%, respectively. These results suggest that the yield of the final product, i.e. α,α-trehalose, becomes higher when such a substrate as having a longer α-1,4-linked straight-chain is used.

### EXAMPLE II-7

### Production of α,α-Trehalose from Soluble Starch in Various Enzyme-Concentrations

Production of α,α-trehalose utilizing the synergism between enzymes was attempted by adding the enzymes having concentrations listed in Table 19, respectively, to a substrate (final concentration: 10%). Specifically, the enzymes were the present purified enzyme obtained in Example II-2, and the purified transferase derived from the Sulfolobus solfataricus strain KM1; the substrate was a soluble starch which had been pre-treated under the conditions of 40° C. for 1 hour with 25 Units/ml of pullulanase (manufactured by Wako pure chemical Co.) derived from Klebsiella pneumoniae; and the reaction was performed under the conditions of 60° C. at pH 5.5 for 100 hours, approximately. After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotridse per minute at pH 6.0 and 30° C. from pullulan assigned for the substrate.

The results are shown in Table 19 below.

TABLE 19
Yield of α,α-trehalose (%)

| Concentration of | | | | | |
|---|---|---|---|---|---|
| amylase | Concentration of transferase (units/ml) | | | | |
| (units/ml) | 0.1 | 1 | 5 | 10 | 20 |
| 1.5 | 7.8 | 28.0 | 9.6 | 8.8 | 9.7 |
| 15 | 10.0 | 45.3 | 34.3 | 33.6 | 35.2 |
| 150 | 8.6 | 51.8 | 59.3 | 62.1 | 65.1 |
| 450 | 1.6 | 45.1 | 58.9 | 61.7 | 64.2 |
| 700 | 1.3 | 19.0 | 39.3 | 44.5 | 46.8 |
| 2000 | 1.7 | 12.9 | 31.5 | 40.3 | 42.7 |

As is obvious from the results shown in the table, the yield of α,α-trehalose reached its maximum, i.e. 65.1%, in such a case with 20 Units/ml of the transferase and 150 Units/ml of the amylase.

### Comparative Example II-2

### Production of α,α-Trehalose Using Amylases Derived from the Other Organisms

Production of α,α-trehalose utilizing the synergism between enzymes was attempted as follows:

Amylases derived from Bacillus subtilis, Bacillus licheniformis and Aspergillus oryzae (100200 manufactured by Seikagaku Kougyou Co, A-3403 and A-0273 manufactured by Sigma Co., respectively; all of them are active at 60° C.) were used as comparative substitutions for the novel amylase of the present invention;

the purified transferase used together was derived from the Sulfolobus solfataricus strain KM1;

the substrate was a soluble starch (final concentration: 10%) which had been pre-treated under the conditions of 40° C. and 1 hour with 25 Units/ml of pullulanase (manufactured by Wako pure chemical Co.) derived from Klebsiella pneumoniae;

the enzymes having concentrations listed in Table 20, respectively, was added to the substrate; and

the reaction was performed under the conditions of 60° C. at pH 5.5 for 100 hours, approximately. After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to enzymatic activity of each amylase, 1 Unit is defined as equalling the amount of the enzyme with which the absorptivity at 620 nm corresponding to the violet color of the starch-iodine complex decreases at a rate of 10% per 10 min. under the same reaction conditions as in Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotriose per minute at pH 6.0 and 30° C. from pullulan assigned for the substrate.

The results are shown in Table 20 below.

TABLE 20
Yield of α,α-trehalose (%)

| Concentration | | Concentration | Yield of |
|---|---|---|---|
| of transferase | | of α-amylase | α,α-trehalose |
| (units/ml) | Origin of α-amylase | (units/ml) | (%) |
| 10 | Bacillus subtilis | 1.0 | 28.9 |
| 10 | | 10.0 | 27.7 |
| 5 | Bacillus licheniformis | 10.0 | 26.4 |
| 10 | | 10.0 | 26.8 |
| 5 | Aspergillus oryzae | 1.0 | 23.2 |
| 10 | | 1.0 | 23.1 |

As is obvious from the results shown in the table, though α,α-trehalose can be produced by using amylases derived from the other organisms, the yield in each case is lower than that in the case using the novel enzyme of the present invention.

### EXAMPLE II-8

### Production of α,α-Trehalose from Amylose DP-17 in Various Enzyme-Concentrations

Production of α,α-trehalose utilizing the synergism between enzymes was attempted by adding the enzymes having concentrations listed in Table 21, respectively, to a substrate (final concentration: 10%). Specifically, the enzymes were the present purified enzyme obtained in Example II-2, and the purified transferase derived from the Sulfolobus solfataricus strain KM1; the substrate was Amylose DP-17 (manufactured by Hayashibara Biochemical Co.); and the reaction was performed under the conditions of 60C at pH 5.5 for 100 hours, approximately. After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

The results are shown in Table 21 below.

TABLE 21
Yield of α,α-trehalose (%)

| Concentration | | | | | |
|---|---|---|---|---|---|
| of amylase | Concentration of transferase (units/ml) | | | | |
| (units/ml) | 0.1 | 1 | 5 | 10 | 20 |
| 1.5 | 11.9 | 46.8 | 52.1 | 48.4 | 40.4 |
| 15 | 25.6 | 77.9 | 79.7 | 81.8 | 77.4 |
| 150 | 10.7 | 62.1 | 76.9 | 83.4 | 81.9 |
| 200 | 2.8 | 47.9 | 73.2 | 76.1 | 79.2 |
| 700 | 1.2 | 17.0 | 49.1 | 61.8 | 68.4 |
| 2000 | 0.6 | 9.2 | 27.5 | 36.7 | 48.7 |

As is obvious from the results shown in the table, when Amylose DP-17, which consists of a straight-chain constructed with α-1,4-linkages, was used as the substrate, the yield of α,α-trehalose reached its maximum, i.e. 83.4%, in such a case with 10 Units/ml of the transferase and 150 Units/ml of the amylase.

### EXAMPLE II-9

### Production of α,α-Trehalose in Various Concentrations of Soluble Starch

Production of α,α-trehalose utilizing the synergism between enzymes was attempted by adding the enzymes having concentrations listed in Table 22, respectively, to a substrate, the final concentration of which would be adjusted at 5%, 10%, 20% or 30%. Specifically, the enzymes were the present purified enzyme obtained in Example II-2, and the purified transferase derived from the Sulfolobus solfataricus strain KM1; the substrate was soluble starch; and the reaction was performed under the conditions of 60° C. at pH 5.5 for 100 hours, approximately. During the reaction, from 0 hours to 96 hours after the start, a treatment at 40° C. for 1 hour with the addition of pullulanase (a product derived from Klebsiella pneumoniae, manufactured by Wako pure chemical Co.) so as to be 5 Units/ml was performed every 12 hours, namely, totaling 9 times inclusive of the treatment at 0 hours.

After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotriose per minute at pH 6.0 and 30° C. from pullulan assigned for the substrate.

The results are shown in Table 22 below.

TABLE 22

| Concentration | Concentration | Concentration | Yield of |
|---|---|---|---|
| of soluble | of transferase | of amylase | α,α-trehalose |
| starch (%) | (units/ml) | (units/ml) | (%) |
| 5 | 2 | 50 | 76.6 |
| | 5 | 150 | 74.4 |
| 10 | 10 | 150 | 77.4 |
| | 20 | 150 | 78.2 |
| 20 | 10 | 150 | 75.7 |
| | 20 | 150 | 75.0 |
| 30 | 10 | 150 | 71.4 |
| | 20 | 150 | 71.9 |

As is obvious from the results shown in the table, the yield of α,α-trehalose can be 70% or more even when the concentration of soluble starch as a substrate was varied in a range of 5-30%, provided that the concentrations of the amylase and transferase are adjusted to the optimum conditions.

### EXAMPLE II-10

### Production of α,α-Trehalose from Soluble Starch with Various Pullulanase Treatments

Production of α,α-trehalose utilizing the synergism between enzymes was attempted as follows:

The enzymes were the present purified enzyme obtained in Example II-2, and the purified transferase derived from the Sulfolobus solfataricus strain KM1;

the substrate was soluble starch (final concentration: 10%);

the enzymes having concentrations listed in Table 23, respectively, was added to the substrate; and

the reaction was performed under the conditions of 60° C. at pH 5.5 for 120 hours, approximately. During the reaction, one or more of pullulanase treatments were performed under either of the following schedules: 1 time at 24 hours after the start (a) (hereinafter, “after the start” will be omitted); 1 time at 48 hours (b); 1 time at 72 hours (c); 1 time at 96 hours (d); every 24 hours from 24 hours to 96 hours, totaling 4 times (e); every 12 hours from 0 hours to 96 hours, totaling 9 times inclusive of the treatment at 0 hours (f); and every 3 hours in the early stage of the reaction, i.e. from 0 hours to 12 hours, totaling 5 times inclusive of the treatment at 0 hours, and in addition, every 12 hours from 24 hours to 96 hours, totaling 7 times (g). Any of the pullulanase treatments were performed under the conditions of 40° C. for 1 hour after the addition of pullulanase (a product derived from Klebsiella pneumoniae) so as to be the concentrations shown in Table 23, respectively.

After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotriose per minute at pH 6.0 and 30° C. from pullulan assigned for the substrate.

The results are shown in Table 23 below.

TABLE 23
Yield of α,α-trehalose (%)

| Method of | Concentration | Concentration | Concentration of pullulanase | | | | | |
|---|---|---|---|---|---|---|---|---|
| Pullulanase | of amylase | of transferase | (units/ml) | | | | | |
| treatment | (units/ml) | (units/ml) | 0.1 | 1 | 2 | 5 | 10 | 25 |
| (a) | 150 | 10 | 48.0 | 59.7 | 62.9 | 67.6 | | 71.7 |
| (b) | 150 | 10 | 49.4 | 60.0 | 62.2 | 66.0 | | 71.0 |
| (c) | 150 | 10 | 49.6 | 59.7 | 63.2 | 66.4 | | 70.0 |
| (d) | 150 | 10 | 49.2 | 59.3 | 62.9 | 67.0 | | 70.0 |
| (e) | 150 | 10 | 57.8 | 69.9 | 72.6 | 74.1 | | |
| (f) | 150 | 10 | | 74.0 | 76.6 | 77.4 | | 67.6 |
| | 150 | 20 | | 74.4 | 74.0 | 78.2 | | 67.0 |
| (g) | 150 | 10 | | 75.7 | 76.5 | 80.9 | 61.9 | |
| | 150 | 20 | | 75.9 | 77.9 | 77.0 | 71.5 | |

As is obvious from the results shown in the table, the yield can be improved by introducing a pullulanase treatment during the reaction. Particularly, the yield of α,α-trehalose can be further improved by a method in which a plurality of pullulanase treatments are carried out, or a method in which a plurality of pullulanase treatments are carried out in the early stage of the reaction. The yield of α,α-trehalose reached its maximum, i.e. 80.9%, under the conditions with 10 Units/ml of the transferase, 150 Units/ml of the amylase, the pullulanase treatment schedule (g), and 5 Units/ml of the pullulanase.

### EXAMPLE II-11

### Production of α,α-Trehalose in Various Concentrations of Amylose DP-17 and Various Reaction Temperatures

Production of α,α-trehalose utilizing the synergism between enzymes was attempted as follows:

The present purified enzyme obtained in Example II-2, and the purified transferase derived from the Sulfolobus solfataricus strain KM1 were added so as to be 320 Units/g-substrate and 20 Units/g-substrate, respectively;

the substrate was Amylose DP-17; and

the reaction was performed for 100 hours, approximately, with the substrate concentration and reaction temperature shown in Table 24 or 25.

After the non-reacted substrate was hydrolyzed with glucoamylase, the reaction mixture was analyzed by the TSK-gel Amide-80 HPLC analyzing method described in Example II-1 to examine the yield of the produced α,α-trehalose and the reaction rate.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

The results are shown in Tables 24 and 25 below.

Incidentally, as to the reaction rate shown in Table 24, 1 Unit is defined as the rate of liberating 1 μmol of α,α-trehalose per hour.

TABLE 24
Reaction rate (units/ml)

| | |
|---|---|
| Reaction | Substrate concentration (%) |

| temperature (° C.) | 10 | 20 | 30 | 40 |
|---|---|---|---|---|
| 40 | 1.1 | 1.8 | 4.8 | 6.2 |
| 50 | 3.2 | 8.1 | 7.7 | 12.3 |
| 60 | 6.8. | 16.2 | 23.8 | 23.1 |
| 70 | 12.0 | 29.3 | 32.3 | 55.6 |
| 80 | 13.3 | 30.8 | 66.9 | 88.0 |

TABLE 25
Reaction yield (%)

| | |
|---|---|
| Reaction | Substrate concentration (%) |

| temperature (° C.) | 10 | 20 | 30 | 40 |
|---|---|---|---|---|
| 40 | 42.7 | 50.3 | 42.6 | 28.8 |
| 50 | 71.0 | 70.2 | 64.6 | 35.2 |
| 60 | 74.6 | 72.5 | 66.2 | 65.8 |
| 70 | 75.1 | 75.0 | 65.4 | 70.7 |
| 80 | 69.3 | 68.2 | 68.4 | 70.9 |

As is obvious from the results shown in the tables, when the reaction temperature is raised to a range of 40-80° C., the reaction rate increases depending on the temperature. Further, with a high substrate concentration (30-40%), the substrate becomes insoluble and the yield markedly decreases when the temperature is low (40-50° C.), while the substrate becomes soluble and the yield can remain high when the temperature is high. The yield reached to 75.1%.

From the results of this example, it can be understood that a preparation at a high temperature in a high concentration will be possible by using the highly thermostable amylase of the present invention, and therefore, a process for producing α,α-trehalose advantageous in view of cost and easy handling can be provided.

### EXAMPLE II-12

### Production of α,α-Trehalose Using Thermostable Pullulanase in Various Concentrations of Soluble Starch and Various Reaction Temperatures

Production of α,α-trehalose utilizing the synergism between enzymes was attempted as follows:

The present purified enzyme obtained in Example II-2, the purified transferase derived from the Sulfolobus solfataricus strain KM1, and a commercially available thermostable pullulanase were added so as to be 1280 Units/g-substrate, 80 Units/g-substrate and 32 Units/g-substrate, respectively, wherein the pullulanase (Debranching Enzyme Amano, a product derived from Bacillus sp. manufactured by Amano Pharmaceutical Co.) had been subjected to TOSHO TSK-gel Phenyl-TOYOPEARL 650S hydrophobic chromatography to remove coexisting glucoamylase activity and α-amylase activity;

the substrate was soluble starch; and

the reaction was performed for 100 hours, approximately, with the substrate concentration and reaction temperature shown in Table 26 or 27.

After the non-reacted substrate was hydrolyzed with glucoamylase, the reaction mixture was analyzed by the TSK-gel Amide-80 HPLC analyzing method described in Example II-1 to examine the yield of the produced α,α-trehalose and the reaction rate.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

As to activity of pullulanase, 1 Unit is defined as the enzymatic activity of producing 1 μmol of maltotriose per minute at pH 5.5 and 60° C. from pullulan assigned for the substrate.

The results are shown in Tables 26 and 27 below.

Incidentally, as to the reaction rate shown in Table 26, 1 Unit is defined as the rate of liberating 1 μmol of α,α-trehalose per hour.

TABLE 26
Reaction rate (units/ml)

| Reaction | Substrate concentration (%) | | |
|---|---|---|---|
| temperature (° C.) | 10 | 20 | 30 |
| 40 | 15.8 | 22.8 | 22.2 |
| 50 | 26.0 | 50.8 | 57.5 |
| 60 | 36.5 | 58.4 | 96.4 |

TABLE 27
Reaction yield (%)

| Reaction | Substrate concentration (%) | | |
|---|---|---|---|
| temperature (° C.) | 10 | 20 | 30 |
| 40 | 53.1 | 8.9 | 6.2 |
| 50 | 70.9 | 56.1 | 58.6 |
| 60 | 74.1 | 72.6 | 71.7 |

Incidentally, when the reaction was performed with a substrate concentration of 10% and a reaction temperature of 60° C. under the same conditions as above except that the thermostable pullulanase was not added, the yield was 35.0%.

From the result shown in the tables, it was found that only one addition of the thermostable pullulanase during the reaction brings about a yield-improving effect, and that the reaction rate increases depending on the temperature when the reaction temperature is raised to a range of 40-60° C. Further, with a high substrate concentration (20-30%), the substrate becomes insoluble and the yield markedly decreases when the temperature is low (40-50° C.), while the substrate becomes soluble and the yield can remain high when the temperature is high (60° C.). The yield reached to 74.1%.

### EXAMPLE II-13

### Production of α,α-Trehalose from Soluble Starch with Isoamylase Treatments

Production of α,α-trehalose utilizing the synergism between enzymes was attempted as follows:

The present purified enzyme obtained in Example II-2, and the purified transferase derived from the Sulfolobus solfataricus strain KM1 were added so as to be 1,280 Units/g-substrate and 80 Units/g-substrate, respectively;

the substrate was soluble starch (final concentration: 10%); and

the reaction was performed at 60° C. and pH 5.0 for 100 hours, approximately. During the reaction, an isoamylase treatment was performed every 3 hours in the early stage of the reaction, i.e. from 0 hours to 12 hours after the start (hereinafter, “after the start” is omitted), totaling 5 times inclusive of the treatment at 0 hours, and in addition, every 24 hours from 24 hours to 96 hours, totaling 3 times. Each isoamylase treatment was performed under the conditions of 40° C. for 1 hour after the addition of isoamylase (a product derived from Pseudomonas amyloderamosa, manufactured by Seikagaku Kougyou Co.) so as to be the concentration shown in Table 28.

After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour. from maltotriosyltrehalose, similar to Example II-1.

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

The activity of isoamylase was measured as follows: A half milliliter of 1% soluble starch derived from glutinous rice was mixed with 0.1 ml of a 0.5 M acetic acid buffer solution (pH 3.5) and 0.1 ml of an enzyme solution, and subjected to reaction at 40° C.; the absorptivity at 610 nm corresponding to the violet color of the amylose-iodine complex is measured with a cuvette having a width of 1 cm [“Denpun-Kanren Toushitsu Kouso Jikken-hou” (“Experimental methods in enzymes for starch and relating saccharides”), written by Michinori Nakamura and Keiji Kainuma, published by Gakkai-Shuppan-Sentah, 1989]; and 1 Unit is defined as the amount of the enzyme with which the absorptivity increases by 0.1 per hour.

The results are shown in Table 28 below.

TABLE 28

| Concentration of | Reaction yield |
|---|---|
| isoamylase (units/ml) | (%) |
| 0 | 35.0 |
| 500 | 75.7 |
| 1000 | 73.7 |
| 2000 | 71.0 |

As is obvious from the results shown in the tables, the yield can be improved by introducing isoamylase treatments during the reaction, similar to pullulanase (a product derived from Klebsiella pneumoniae). The yield of α,α-trehalose reached to 75.7%.

### EXAMPLE II-14

### Production of α,α-Trehalose from Soluble Starch with a Treatment Using a Debranching Enzyme Derived from the Sulfolobus solfataricus strain KM1

Production of α,α-trehalose utilizing the synergism between enzymes was attempted as follows:

The present purified enzyme obtained in Example II-2, the purified transferase derived from the Sulfolobus solfataricus strain KM1, and a debranching enzyme derived from the Sulfolobus solfataricus strain KM1 (the enzyme isolated and purified from the cell extract according to the method in Referential Example II-3) were added so as to be 1,280 Units/g-substrate, 80 Units/g-substrate, and the concentration shown in the below-described table, respectively;

the substrate was soluble starch (final concentration: 10%); and

the reaction was performed at 60° C. and pH 5.0 for 100 hours, approximately.

After the non-reacted substrate was hydrolyzed with glucoamylase, the reaction mixture was analyzed by the TSK-gel Amide-80 HPLC analyzing method described in Example II-1 to examine the yield of the produced α,α-trehalose.

As to activity of the novel amylase of the present invention, 1 Unit is defined as the enzymatic activity of liberating 1 μmol of α,α-trehalose per hour from maltotriosyltrehalose, similar to Example II-1.

As to activity of the purified transferase derived from the Sulfolobus solfataricus strain KM1, 1 Unit is defined as the enzymatic activity of producing. 1 μmol of glucosyltrehalose per hour at pH 5.5 and 60° C. from maltotriose assigned for the substrate.

The activity of the debranching enzyme derived from the Sulfolobus solfataricus strain KM1 was measured as follows: A half milliliter of 1% soluble starch derived from glutinous rice was mixed with 0.1 ml of a 0.5 M acetic acid buffer solution (pH 5.0) and 0.1 ml of an enzyme solution, and subjected to reaction at 60° C.; the absorptivity at 610 nm corresponding to the violet color of the amylose-iodine complex is measured with a cuvette having a width of 1 cm; and 1 Unit is defined as the amount of the enzyme with which the absorptivity increases by 0.1 per hour.

The results are shown in Table 29 below.

TABLE 29

| Concentration of | |
|---|---|
| debranching enzyme | Reaction yield |
| (units/ml) | (%) |
| 0 | 35.0 |
| 3 | 69.8 |
| 6 | 69.5 |
| 12 | 68.0 |
| 24 | 67.8 |

As is obvious from the results shown in the tables, the yield can be improved by only one addition of the debranching enzyme derived from the Sulfolobus solfataricus strain KM1. during the reaction, similar to pullulanase (Debranching Enzyme Amano, a product derived from Bacillus sp.). The yield of α,α-trehalose reached to 69.8%.

### Referential Example II-1

### Production of Transferred Oligosaccharide by Transferase in Various Concentrations of Amylose DP-17 and Various Reaction Temperatures

Using Amylose DP-17 as a substrate, the corresponding trehaloseoligosaccharide, of which the glucose residue at the reducing end side is α-1,α-1-linked, was produced by adding the purified transferase derived from the Sulfolobus solfataricus strain KM1 so as to be 20 Units/g-substrate, and by performing the reaction in the substrate concentration and reaction temperature shown in Table 30 or 31 for 100 hours, approximately.

As to the corresponding trehaloseoligosaccharide, of which the glucose residue at the reducing end is α-1,α-1-linked, the yield and the reaction rate were estimated from the decrement in the amount of reducing ends which was measured by the dinitrosalicylate method [“Denpun-Kanren Toushitsu Kouso Jikken-hou” (“Experimental methods in enzymes for starch and relating saccharides”), written by Michinbri Nakamura and Keiji Kainuma, published by Gakkai-Shuppan-Sentah, 1989].

As to activity of the purified transferase derived from the Sulfolobus solfataricus strain KM1, 1 Unit is defined as the enzymatic activity of producing 1 μmol of glucosyltrehalose per hour at pH 5.5 and 60° C. from maltotriose assigned for the substrate.

The results are shown in Tables 30 and 31 below.

Incidentally, as to the reaction rate shown in Table 30, 1 Unit is defined as the rate of liberating 1 μmol of α,α-trehalose per hour.

TABLE 30
Reaction rate (units/ml)

| | |
|---|---|
| Reaction | Substrate concentration (%) |

| temperature (° C.) | 10 | 20 | 30 | 40 |
|---|---|---|---|---|
| 40 | 0.8 | 2.9 | 3.5 | 4.3 |
| 50 | 3.0 | 5.5 | 8.6 | 8.1 |
| 60 | 1.7 | 6.5 | 10.3 | 16.7 |
| 70 | 4.0 | 7.0 | 12.0 | 19.8 |
| 80 | 3.6 | 9.4 | 15.8 | 20.4 |

TABLE 31
Reaction yield (%)

| | |
|---|---|
| Reaction | Substrate concentration (%) |

| temperature (° C.) | 10 | 20 | 30 | 40 |
|---|---|---|---|---|
| 40 | 70.7 | 74.5 | 63.4 | 37.6 |
| 50 | 76.0 | 72.8 | 70.5 | 46.7 |
| 60 | 71.6 | 75.1 | 75.3 | 55.1 |
| 70 | 71.6 | 70.4 | 76.6 | 72.6 |
| 80 | 65.6 | 64.8 | 72.7 | 72.5 |

From the result shown in the tables, it was found that the reaction rate increases depending on the temperature when the reaction temperature is raised to a range of 40-80° C. Further, with a high substrate concentration (especially 40%), the substrate becomes insoluble and the yield markedly decreases when the temperature is low (40-50° C., while the substrate becomes soluble and the yield can remain high when the temperature is high. The yield reached to 76.6%.

### Referential Example II-2

### Measuring Solubility of Amylose DP-17 in Water

Solubility of Amylose DP-17 was measured as follows: By heat dissolution, 5, 10, 20, 30 and 40% Amylose DP-17 solutions were prepared, and kept in thermostat baths adjusted at 35, 40, 50, 70 and 80° C., respectively; time-lapse sampling was performed and the insoluble matters generated in the samples were filtered; each of the supernatants thus obtained was examined for the concentration of Amylose DP-17; and the solubility at each temperature was determined according to the saturation point where the concentration had been reached to equilibrium.

The results are shown in Table 32 below.

TABLE 32

| Temperature | Solubility |
|---|---|
| (° C.) | (% (w/vol)) |
| 35 | 11.3 |
| 40 | 13.0 |
| 50 | 18.9 |
| 60 | 27.6 |
| 70 | 32.3 |
| 80 | 35.3 |

### Referential Example II-3

### Purification of the Debranching Enzyme Derived from the Sulfolobus solfataricus strain KM1

The Sulfolobus solfataricus strain KM1 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 3.3 g/liter. Eighty two grams of the bacterial cells obtained above were suspended in 400 ml of a 50 mM sodium acetate buffer solution (pH 5.5) containing 5 mM of EDTA, and subjected to ultrasonic treatment for bacteriolysis at 0° C. for 15 min. The resultant was then centrifuged to obtain a supernatant.

To this supernatant, ammonium sulfate was added so as to be 1 M. The resultant was then subjected to hydrophobic chromatography using TOSOH TSK-gel Phenyl-TOYOPEARL 650S column (volume: 800 ml) equilibrated with a 50 mM sodium acetate buffer solution (pH 5.5) containing 1 M of sodium sulfate and 5 mM of EDTA. The column was then washed with the same buffer solution, and the debranching enzyme was recovered in the fraction passing through the column. Since amylase, transferase and glucoamylase contained in the supernatant were retained and adsorbed in the packed material of the column, Phenyl-TOYOPEARL 650S, the objective debranching enzyme could be separated therefrom. The fraction exhibiting the activity was concentrated using an ultrafiltration membrane (critical molecular weight: 13,000), and subsequently, washed and desalted with a 10 mM Tris-HCl buffer solution (pH 7.5).

Next, the resultant was subjected to ion-exchange chromatography using the TOSOH TSK-gel DEAE-TOYOPEARL 650S column (volume: 300 ml) equilibrated with the same buffer solution. The column was then washed with the same buffer solution, and the objective debranching enzyme was then eluted with 900 ml of sodium chloride solution at a linear concentration gradient from 0 M to 0.3 M. The fractions exhibiting the activity were concentrated using an ultrafiltration membrane (critical molecular weight: 13,000), and subsequently, washed and desalted with a 50 mM sodium acetate buffer solution (pH 5.5) containing 0.15 M of sodium chloride and 5 mM of EDTA.

Subsequent to that, the desalted and concentrated solution thus obtained was subjected to gel filtration chromatography using the Pharmacia HiLoad 16/60 Superdex 200pg column, and the objective debranching enzyme was eluted with the same buffer solution. The fractions exhibiting the activity were concentrated using an ultrafiltration membrane (critical molecular weight: 13,000), and subsequently, washed and desalted with a 25 mM bis-Tris-iminodiacetic acid buffer solution (pH 7.1).

Next, the desalted and concentrated solution thus obtained was subjected to a chromatofocusing using the Pharmacia Mono P HR5/20 column equilibrated with the same buffer solution. The objective debranching enzyme was then eluted with 10% Polybuffer 74 (manufactured by Pharmacia, and adjusted at pH 4.0 with iminodiacetic acid). The fractions exhibiting the activity were concentrated using an ultrafiltration membrane (critical molecular weight: 13,000), and subsequently, washed and desalted with a 10 mM Tris-HCl buffer solution (pH 7.5).

Further, the desalted and concentrated solution thus obtained was subjected to ion-exchange chromatography using the TOSOH TSK-gel DATE 5PW HPLC column equilibrated with the same buffer solution. The column was then washed with the same buffer solution, and the objective debranching enzyme was then eluted with 30 ml of sodium chloride solution at a linear concentration gradient from 0 M to 0.3 M. The fractions exhibiting the activity were concentrated using an ultrafiltration membrane (critical molecular weight: 13,000) to obtain the partially purified product (liquid product) of the objective debranching enzyme.

Incidentally, in this purification procedure, detection of the objective debranching enzyme was performed by mixing the sample solution with 2 Units/ml of the purified amylase and 32 Units/ml of the purified transferase derived from the Sulfolobus solfataricus strain KM1, and by putting the mixture into a reaction at 60° C. and pH 5.5, wherein the index was the activity of achieving a higher yield of α,α-trehalose in comparison with the reaction without the sample solution.

The activity of the partially purified debranching enzyme, obtained by the above-described purification process and derived from the Sulfolobus solfataricus strain KM1, was measured as follows: A half milliliter of 1% soluble starch derived from glutinous rice was mixed with 0.1 ml of a 0.5 M acetic acid buffer solution (pH 5.0) and 0.1 ml of an enzyme solution, and subjected to reaction at 60° C.; the absorptivity at 610 nm corresponding to the violet color of the amylose-iodine complex is measured with a cuvette having a width of 1 cm; and 1 Unit is defined as the amount of the enzyme with which the absorptivity increases by 0.1 per hour.

The specific activity of the partially purified debranching enzyme obtained by the above purification procedure was found to be 495 Units/mg.

### Referential Example II-4

### Examination of the Debranching Enzyme Derived from the Sulfolobus solfataricus strain KM1 for Various Characteristics

The partially purified debranching enzyme obtained in Referential. Example II-3 was examined for enzymatic characteristics.

(1) Action and Substrate Specificity

The reactivity and action on each substrate were examined using each the substrate and activity-measuring methods shown in Table 33 below.

The dinitrosalicylate method [“Denpun-Kanren Toushitsu Kouso Jikken-hou” (“Experimental methods in enzymes for starch and relating saccharides”), written by Michinori Nakamura and Keiji Kainuma, published by Gakkai-Shuppan-Sentah, 1989] is a method for quantifying the increased amount of reducing ends generated by hydrolysis of α-1,6 linkages.

The iodine-coloring method is carried out in the same way as described in Referential Example II-3. Specifically, this is the method for quantifying the increased amount of straight-chain amylose generated by hydrolysis of α-1,6 linkages on the basis of increased absorptivity at 610 nm corresponding to the violet color of the amylose-iodine complex.

Analysis of the hydrolyzed products by liquid chromatography (HPLC method) was performed for examination of the produced oligosaccharides by employing the Bio-Rad AMINEX HPX-42A HPLC analyzing method described in Example II-1.

TABLE 33

| | Method of enzyme assay | | |
|---|---|---|---|
| | Dinitrosalicylate | Iodine-coloring | HPLC |
| Substrate | method | method | method |
| Pullulan | +++ | − | Maltotriose |
| Soluble starch | + | + | − |
| Amylopectin | + | + | − |
| Glutinous rice | + | + | − |
| starch | | | |

As is obvious from the above results, the present debranching enzyme can 1) generate reducing ends in pullulan and various kinds of starch; 2) increase the coloring degree in the iodo-starch reaction; 3) produce maltotriose from pullulan; and further, 4) as shown in Example II-14, markedly increase the yield of α,α-trehalose from soluble starch used as a substrate when the present debranching enzyme is put into the reaction with the purified amylase and transferase derived from the Sulfolobus solfataricus strain KM1, as compared with the reaction without the addition of the present debranching enzyme. As a consequence of these facts, the present enzyme is recognized as hydrolyzing α-1,6 linkages in starch or pullulan.

(2) Stability

The stability of the obtained partially purified enzyme when treated at various temperatures for 3 hours is shown in Table 34.

TABLE 34

| Temperature | Residual activity |
|---|---|
| (° C.) | (%) |
| 50 | 109.1 |
| 60 | 73.3 |
| 65 | 6.1 |
| 70 | 0 |

The present enzyme treated at 60° C. for 3 hours still retains 73.3% of the initial activity.

(3) Reactivity

As to the obtained partially purified enzyme, reactivity at various temperatures and reactivity at various pH values are shown in Tables. 35 and 36, respectively. In the measurement of enzymatic activity, a glycine-HCl buffer solution was used in a pH range of 3-5, and similarly, a sodium acetate.buffer solution in a pH range of 4-5.5, and a sodium phosphate buffer solution in a pH range of 5-7.5, respectively, were also used.

TABLE 35

| | Relative enzyme |
|---|---|
| Reaction pH | activity (%) |
| 2.7 | 1.8 |
| 3.1 | 21.7 |
| 3.7 | 33.1 |
| 4.1 | 74.0 |
| 5.1 | 100.0 |
| 5.5 | 53.7 |
| 5.6 | 37.5 |
| 6.0 | 22.2 |
| 6.9 | 16.1 |
| 7.4 | 11.5 |
| 7.7 | 10.2 |

TABLE 36

| Reaction temperature | Relative enzyme |
|---|---|
| (° C.) | activity (%) |
| 40 | 53.8 |
| 50 | 87.0 |
| 60 | 97.6 |
| 65 | 100.0 |
| 70 | 51.4 |

The optimum reaction temperature of the present enzyme is within 60-65° C., approximately, and the optimum reaction pH of the present enzyme is within 4.0-5.5, approximately.

(4) Isoelectric Point

The isoelectric point was found to be pH 4.4 from the result of pH measurement performed on the debranching enzyme fraction isolated by chromatofocusing.

(5) Influence of Various Activators and Inhibitors

The influence of each substance listed in Table 37, such as an activating effect or an inhibitory effect, was evaluated by adding the substance together with the substrate, and by measuring the activity in the same manner as that in Referential Example II-3. As a result, copper ion was found to have inhibitory effects. Though many glucide-relating enzymes have been found to be activated with calcium ion, the present enzyme would not be activated with calcium ion.

TABLE 37

| | Concentration | Residual activity |
|---|---|---|
| Activator/Inhibitor | (mM) | (%) |
| Control (not added) | 5 | 100.0 |
| CaCl₂ | 5 | 105.7 |
| MgCl₂ | 5 | 82.9 |
| MnCl₂ | 5 | 91.2 |
| CuSO₄ | 5 | 0.0 |
| CoCl₂ | 5 | 87.2 |
| FeSO₄ | 5 | 74.1 |
| FeCl₃ | 5 | 39.0 |
| 2-Mercaptoethanol | 5 | 104.1 |
| Dithiothreitol | 5 | 106.0 |

### EXAMPLE I-9

### Determination of the Partial Amino Acid Sequences of the Novel Transferase Derived from the Sulfolobus solfataricus strain KM1

The partial amino acid sequences of the purified enzyme obtained in Example I-2 were determined by the method disclosed in Iwamatsu, et al. [Seikagaku (Biochemistry) 63, 139 (1991)]. Specifically, the purified novel transferase was suspended in a buffer solution for electrophoresis [10% glycerol, 2.5% SDS, 2% 2-mercaptoethanol, 62 mM Tris-HCl buffer solution (pH 6.8)], and subjected to SDS-polyacrylamide gel electrophoresis. After the electrophoresis, the enzyme was transferred from the gel to a polyvinylidene diflorido (PVDF) membrane (ProBlot, manufactured by Applied Biosystems Co.) by electroblotting (SartoBlot type IIs, manufactured by Sartorius Co.) with 160 mA for 1 hour.

After the transfer, the portion to which the enzyme had been transferred was cut out from the membrane, and soaked in about 300 μl of a buffer solution for reduction [6 M guanidine-HCl, 0.5 M Tris-HCl buffer solution (pH 3.5) containing 0.3% of EDTA and 2% of acetonitrile]. One milligram of dithiothreitol was added to this, and reduction was carried out under an argon atmosphere at 60° C. for 1 hour, approximately. To the resultant, 2.4 mg of monoiodoacetic acid dissolved in 10 μl of 0.5 N sodium hydroxide was added and stirred for 20 min. in the dark. The PVDF membrane was then taken out and washed sufficiently with a 2% acetonitrile solution, and subsequently, stirred in a 0.1% SDS solution for 5 min. After being briefly washed with water, the PVDF membrane was then soaked in 0.5% Polyvinylpyrrolidone-40 dissolved in 100 mM acetic acid, and was left standing for 30 min. Next, the PVDF membrane was briefly washed with water and cut into pieces of 1 square mm, approximately. These pieces were then soaked in a buffer solution for digestion [8% acetonitrile, 90 mM Tris-HCl buffer solution (pH 9.0)], and after the addition of 1 μmol of the Achromobacter Protease I (manufactured by Wako pure chemical Co.), digested at room temperature for 15 hours. The digested products were separated by reversed phase chromatography using a C8 column (μ-Bondashere 5C8, 300A, 2.1×150 mm, manufactured by Millipore Ltd. Japan) to obtain a dozen or more kinds of peptide fragments. Using A solvent (0.05% trifluoroacetic acid) and B solvent (2-propanol:acetonitrile=7:3, containing 0.02% of trifluoroacetic acid) as elution solvents, the peptides were eluted with a linear concentration gradient from 2 to 50% relative to B solution and at a flow rate of 0.25 ml/min. for 40 min. As to the peptide fragments thus obtained, the amino acid sequences were determined by the automatic Edman degradation method using a gas-phase peptide sequencer (Model 470 type, manufactured by Applied Biosystems Co.).

Further, the peptide fragments digested with the Achromobacter Protease I were subjected to second digestion with Asp-N, and the resultant peptide fragments were isolated under the same conditions as above to determine their amino acid sequences.

From the results, the partial amino acid sequences were found to be as follows.

Peptide Fragments Digested with Achromobacter
Protease

| | | |
|---|---|---|
| AP-1: | Val Ile Arg Glu Ala Lys | (Sequence No. 9) |
| AP-2: | Ile Ser Ile Arg Gln Lys | (Sequence No. 10) |
| AP-3: | Ile Ile Tyr Val Glu | (Sequence No. 11) |
| AP-4: | Met Leu Tyr Val Lys | (Sequence No. 12) |
| AP-5: | Ile Leu Ser Ile Asn Glu | (Sequence No. 13) |
| | Lys | |
| AP-6: | Val Val Ile Leu Thr Glu | (Sequence No. 14) |
| | Lys | |
| AP-7: | Asn Leu Glu Leu Ser Asp | (Sequence No. 15) |
| | Pro Arg Val Lys | |
| AP-8: | Met Ile Ile Gly Thr Tyr | (Sequence No. 16) |
| | Arg Leu Gln Leu Asn Lys | |
| AP-9: | Val Ala Val Leu Phe Ser | (Sequence No. 17) |
| | Pro Ile Val | |
| AP-10: | Ile Asn Ile Asp Glu Leu | (Sequence No. 18) |
| | Ile Ile Gln Ser Lys | |
| AP-11: | Glu Leu Gly Val Ser His | (Sequence NO. 19) |
| | Leu Tyr Leu Ser Pro Ile | |

Peptide Fragments Digested with Asp-N

| | | |
|---|---|---|
| DN-1: | Asp Glu Val Phe Arg Glu | (Sequence No. 20) |
| | Ser | |
| DN-2: | Asp Tyr Phe Lys | (Sequence No. 21) |
| DN-3: | Asp Gly Leu Tyr Asn Pro | (Sequence No. 22) |
| | Lys | |
| DN-4: | Asp Ile Asn Gly Ile Arg Glu | (Sequence No. 23) |
| | Cys | |
| DN-5: | Asp Phe Glu Asn Phe Glu | (Sequence No. 24) |
| | Lys | |
| DN-6: | Asp Leu Leu Arg Pro Asn | (Sequence No. 25) |
| | Ile | |
| DN-7: | Asp Ile Ile Glu Asn | (Sequence No. 26) |
| DN-8: | Asp Asn Ile Glu Tyr Arg | (Sequence No. 27) |
| | Gly | |

### EXAMPLE I-10

### Preparation of Chromosome DNA of the Sulfolobus solfataricus strain KM1

Bacterial cells of the Sulfolobus solfataricus strain KM1 were obtained according to the process described in Example I-2.

To 1 g of the bacterial cells, 10 ml of a 50 mM Tris-HCl buffer solution (pH 8.0) containing 25% of sucrose, 1 mg/ml of lysozyme, 1 mM of EDTA, and 150 mM of NaCl was added for making a suspension, and the suspension was left standing for 30 min. To this suspension, 0.5 ml of 10% SDS and 0.2 ml of 10 mg/ml Proteinase K (manufactured by Wako pure chemical Co.) were added, and the mixture was left standing at 50° C. for 2 hours. Next, the mixture was subjected to extraction with a phenol/chloroform solution. The resultant aqueous phase was then separated and precipitated with ethanol. The precipitated DNA was twisted around a sterilized glass stick and vacuum-dried after being washed with a 70% ethanol solution. As the final product, 1.5 mg of the chromosome DNA was obtained.

### EXAMPLE I-11

### Preparation of DNA Probes Based on the Partial Amino Acid Sequences and Evaluation of the Probes by PCR Method

According to information about the partial amino acid sequences of the novel transferase derived from the Sulfolobus solfataricus strain KM1, which is determined in Example I-9, oligonucleotide DNA primers are prepared by using a DNA synthesizer (Model 381 manufactured by Applied Biosystems Co.). Their sequence were as follows.

DN-1

Amino Acid Sequence

| | | |
|---|---|---|
| N terminus | AspGluPheArgGluSer | C terminus |
| DNA Primer | 5′ TTCACGAAAAACCTCATC 3′ | (Sequence No. 28) |
| Base Sequence | C T TG T T | |

DN-8

Amino Acid Sequence

| | | |
|---|---|---|
| N terminus | AspAsnIleGluTyrArgGly | C terminus |
| DNA Primer | 5′ GATAACATAGAATACAGAGG 3′ | (Sequence No. 29) |
| Base Sequence | T T G T G | |

PCR was performed using 100 pmol of each primer and 100 ng of the chromosome DNA prepared in Example I-10 and derived from the Sulfolobus solfataricus strain KM1. The PCR apparatus used herein was the GeneAmp PCR system Model 9600, manufactured by Perkin Elmer Co. In the reaction, 30 cycles of steps were carried out with 100 μl of the total reaction mixture, wherein the 1 cycle was composed of steps at 94° C. for 30 sec., at 50° C. for 1 min., and at 72° C. for 2 min.

Ten microliters of the resultant reaction mixture was analyzed by 1% agarose electrophoresis. As a result, it was found that a DNA fragment having a length of-about 1.2 kb was specifically amplified.

The product obtained by the above PCR were blunt-ended, and subcloned into pUC118 at the Hinc II site. The DNA sequence of the insertional fragment in this plasmid was determined using a DNA sequencer, GENESCAN Model 373A manufactured by Applied Biosystems Co. As a result, the DNA sequence was found to correspond to the amino acid sequence obtained in Example I-9.

### EXAMPLE I-12

### Cloning of a Gene Coding for the Novel Transferase Derived from the Sulfolobus solfataricus Strain KM1

One hundred micrograms of the chromosome DNA of the Sulfolobus solfataricus strain KM1, prepared in Example I-10, was partially digested with a restriction enzyme, Sau 3AI. The reaction mixture was ultracentrifuged with a density gradient of sucrose to isolate and purify DNA fragments of 5-10 kb. Then, using T4 DNA ligase, the above chromosome DNA fragments having lengths of 5-10 kb and derived from the Sulfolobus solfataricus strain KM1 were ligated with a modified vector which had been prepared from a plasmid vector, pUC118, by digestion with Bam HI and by dephosphorylation of the ends with alkaline phosphatase. Next, cells of the E. coli strain JM109 were transformed with a mixture containing the modified pUC118 plasmid vectors in which any of the fragments had been inserted. These cells were cultivated on LB agar plates containing 50 μg/ml of ampicillin to grow their colonies and make a DNA library.

As to this DNA library, screening of the, recombinant plasmids containing a gene coding for the novel transferase was performed employing a PCR method as follows.

At first, the colonies were scraped and suspended in a TE buffer solution. The suspension was then treated at 100° C. for 5 min. to crush the bacterial bodies and subjected to PCR in the same manner as described in Example I-11.

Next, 10 μl of the reaction mixture obtained in PCR was analyzed by 1% agarose electrophoresis, and the clones from which a DNA fragment having a length of about 1.2 kb can be amplified were assumed to be positive.

As a result, one positive clone was obtained from 600 of the transformants. According to analysis of the plasmid extracted from the clone, it had an insertional fragment of about 8 kb. This plasmid was named as pKT1.

Further, the insertional fragment was shortened by subjecting it to partial digestion with Sau 3AI and PCR in the same manner as above. As a result, such transformants as containing plasmids which have insertional fragments of about 3.-8 kb and about 4.5 kb were obtained. These plasmids were named as pKT21 and pKT11, respectively.

The restriction maps of insertional fragments of these plasmids are shown in FIG. 26.

Incidentally, all the restriction enzymes used in the above examples were commercially available (purchased from Takara Shuzou Co.).

### EXAMPLE I-13

### Determination of the Gene Coding for the Novel Transferase Derived from the Sulfolobus solfataricus strain KM1

The base sequence of the partial DNA which is common both in the insertional fragments, the plasmids pKT11 and pKT21 obtained in Example I-12, was determined.

At first, deletion plasmids were prepared from these plasmid DNAs by using a deletion kit for kilo-sequencing which was manufactured by Takara Shuzou Co. After that, the DNA sequences of the insertional fragments in these plasmids were determined by using a sequenase dye primer sequencing kit, PRISM, a terminator cycle sequencing kit, Tag Dye Deoxy™, both manufactured by Perkin Elmer Japan Co., and a DNA sequencer, GENESCAN Model 373A, manufactured by Applied Biosystems Co.

Among the common sequence, the base sequence from the Sph I site to an end of pKT21 (from A to B in FIG. 26), and the amino sequence anticipated therefrom are shown in Sequences No. 1 and No. 2, respectively.

Sequences corresponding to any of the partial amino acid sequences obtained in Example I-9, respectively, were recognized in the above amino acid sequence. This amino acid sequence was assumed to have 728 amino acid residues and code for a protein, the molecular weight of which estimated as 82 kDa. This molecular weight value almost equals the value obtained by SDS-PAGE analysis of the purified novel transferase derived from the Sulfolobus solfataricus strain KM1.

### EXAMPLE I-14

### Production of the Novel Transferase in a Transformant

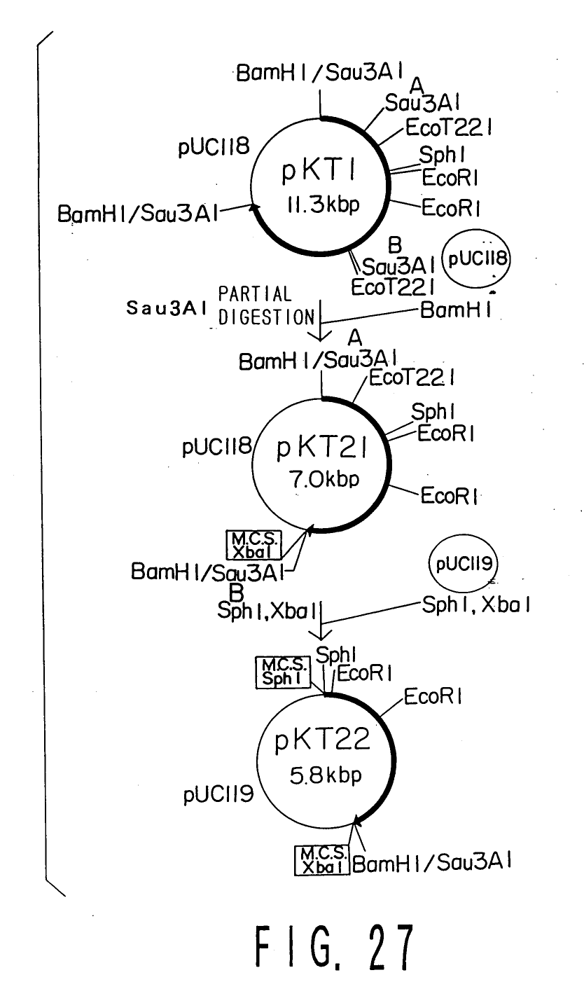

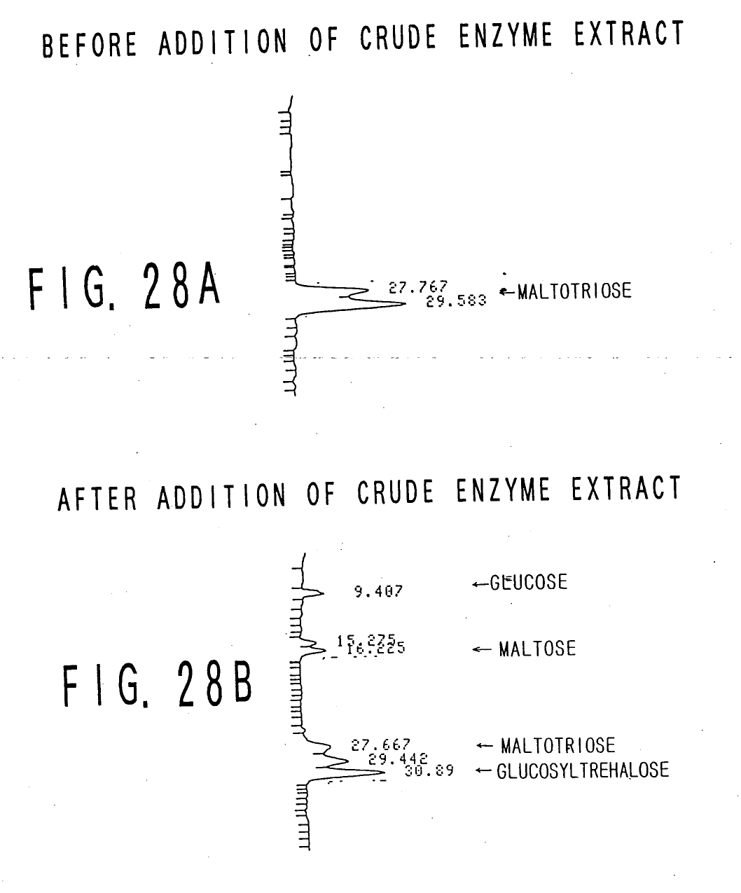

A plasmid named as pKT22 was obtained by restricting pKT21, which was obtained in Example I-12, with Sph I and Xba I, and by ligating the resultant with pUC119 (manufactured by Takara Shuzou Co.) which had been restricted with the same restriction enzymes(the methods are shown in FIG. 27). Except for the multi-cloning site, the base sequence of the fragment which was inserted into pKT22 and contains the novel transferase gene equaled the sequence from the 1st base to the 2578th base of Sequence No. 1.

The activity of the novel transferase in the transformant containing this plasmid was examined as follows. At first, the transformant was cultivated overnight in a LB broth containing 100 μg/ml of ampicillin at 37° C. The cells were collected by centrifugation and stored at −80° C. The yield of bacterial cells was 10 g/liter.

Ten grams of the bacterial cells obtained above were then suspended in 40 ml of a 50 mM sodium acetate buffer solution (pH 5.5) containing 5 mM of EDTA, subjected to bacteriolysis with an ultrasonic crushing-treatment at 0° C. for 3 min., and further, centrifuged to obtain a supernatant. This supernatant was heat-treated at 75° C. for 30 min., further centrifuged, and then concentrated with an ultrafiltration membrane (critical molecular weight: 13,000) to produce a crude enzyme solution (6 Units/ml). Maltotriose, as a substrate, was added so that the final concentration would be 10%. The reaction was carried out at pH 5.5 (50 mM sodium acetate) and at 60° C. for 24 hours, and stopped by heat-treatment at 100° C. for 5 min. The produced glucosyltrehalose was analyzed by the same HPLC analyzing method used in Example I-1.

The results of the HPLC analysis are shown in FIG. 28. The principal reaction-product appeared in the HPLC chart as a peak without any anomers, exhibiting such a retention time as slightly behind the non-reacted substrate. Further, the principal product was isolated using a TSK-gel Amide-80 HPLC column, and analyzed by ¹H-NMR and ¹³C-NMR to be confirmed as glucosyltrehalose.

Consequently, the transformant was found to have the activity of the novel transferase derived from the Sulfolobus solfataricus strain KM1. Incidentally, no activity of the novel transferase was detected in the transformant prepared by transforming the JM109 with pUC119 alone.

### EXAMPLE I-15

### Determination of Partial-Amino Acid Sequences of the Novel Transferase Derived from the Sulfolobus solfataricus strain KM1

Partial amino acid sequences of the novel transferase obtained in Example I-4 were determined according to the process described in Example I-9. The following are the determined partial amino acid sequences.

Peptide Fragments Digested with Achromobacter
Protease

| | | |
|---|---|---|
| AP-6: | Arg Asn Pro Glu Ala Tyr | (Sequence No. 30) |
| | Thr Lys | |
| AP-8: | Asp His Val Phe Gln Glu | (Sequence No. 31) |
| | Ser His Ser | |
| AP-10: | Ile Thr Leu Asn Ala Thr | (Sequence No. 32) |
| | Ser Thr | |
| AP-12: | Ile Ile Ile Val Glu Lys | (Sequence No. 33) |
| AP-13: | Leu Gln Gln Tyr Met Pro | (Sequence No. 34) |
| | Ala Val Tyr Ala Lys | |
| AP-14: | Asn Met Leu Glu Ser | (Sequence No. 35) |
| AP-16: | Lys Ile Ser Pro Asp Gln | (Sequence No. 36) |
| | Phe His Val Phe | |
| | Asn Gln Lys | |
| AP-18: | Gln Leu Ala Glu Asp Phe | (Sequence No. 37) |
| | Leu Lys | |
| AP-19: | Lys Ile Leu Gly Phe Gln | (Sequence No. 38) |
| | Glu Glu Leu Lys | |
| AP-20: | Ile Ser Val Leu Ser Glu | (Sequence No. 39) |
| | Phe Pro Glu Glu | |
| AP-23: | Leu Lys Leu Glu Glu Gly | (Sequence No. 40) |
| | Ala Ile Tyr | |
| AP-28: | Glu Val Gln Ile Asn Glu | (Sequence No. 41) |
| | Leu Pro | |

Peptide Fragments Digested with Asp-N

| | | |
|---|---|---|
| DN-1: | Asp His Ser Arg Ile | (Sequence No. 42) |
| DN-5: | Asp Leu Arg Tyr Tyr Lys | (Sequence No. 43) |
| DN-6: | Asp Val Tyr Arg Thr Tyr | (Sequence No. 44) |
| | Ala Asn Gln Ile Val Lys | |
| | Glu Cys | |

### EXAMPLE I-16

### Cloning of a Gene Coding for the Novel Transferase Derived from the Sulfolobus acidocaldarius strain ATCC 33909

The chromosome DNA of the Sulfolobus acidocaldarius strain ATCC 33909 was obtained according to the process described in Example I-10 from bacterial cells obtained according to the process described in Example I-4. The above chromosome DNA was partially digested with Sau 3AI and subsequently, ligated to a Bam HI-restricted arm of EMBL3 (manufactured by STRATAGENE Co.) by using T4 DNA ligase. Packaging was carried out using Gigapack II Gold, manufactured by STRATAGENE Co. With the library obtained above, the E. coli strain LE392 was infected at 37° C. for 15 min., inoculated on NZY agar plates, and incubated at 37° C. for 8-12 hours, approximately, to form plaques. After being stored at 4° C. for about 2 hours, DNA was adsorbed on a nylon membrane (Hybond N+, manufactured by Amersham Co. Baking was performed at 80° C. for 2 hours after brief washing with 2 ×SSPE. Using the Eco RI-Xba I fragment (corresponding to the sequence from the 824th base to the 2578th base of Sequence No. 1) of pKT22 obtained in Example I-14, the probe was labeled with ³²p employing Megaprime DNA labeling system manufactured by Amersham Co.

Hybridization was performed overnight under the conditions of 60° C. with 6×SSPE containing 0.5% of SDS. Washing was performed by treating twice with 2×SSPE containing 0.5% of SDS at room temperature for 10 min.

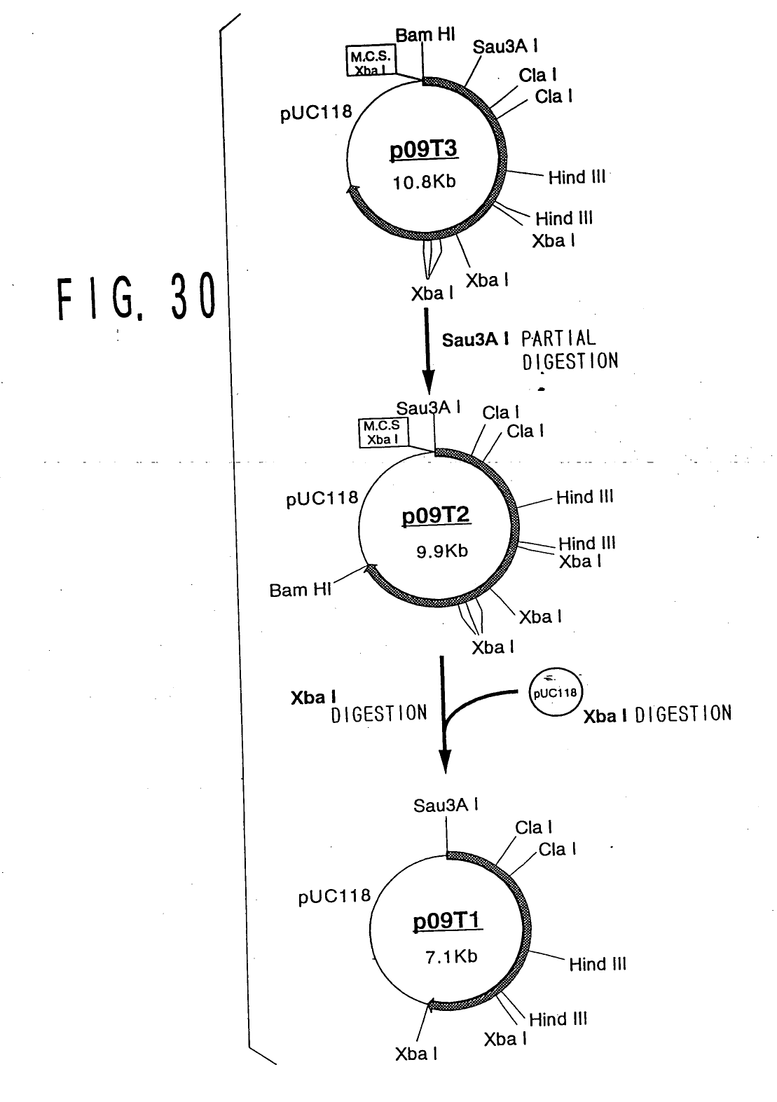

Screening was started with 5,000 clones, approximately, and 8 positive clones were obtained. From these clones, a Bam HI fragment of about 7.6 kbp was obtained and the fragment was inserted into pUC118 at the corresponding restriction site. The plasmid thus obtained was named as p09T3. Further, the insertional fragments of the above clones were partially digested with Sau 3AI and the obtained fragment of about 6.7 kbp was inserted into pUC118 at the Bam HI site. The plasmid thus obtained was named as pO9T2. The Xba I fragment which was derived from this plasmid and had about 3.8 kbp was inserted into pUC118 at the corresponding restriction site. The plasmid thus obtained was named as pO9T1. The restriction map of this plasmid is shown in FIG. 29, and the preparation procedure thereof is shown in FIG. 30. As to the above plasmid pO9T1, the base sequence, principally of the region coding for the novel transferase, was determined according to the process described in Example I-13. The base sequence thus determined and the amino acid sequence anticipated therefrom are shown in Sequences No. 3 and No. 4, respectively. Sequences corresponding to any of the partial amino acid sequences obtained in Example I-15, respectively, were recognized in this amino acid sequence. This amino acid sequence was assumed to have 680 amino acid residues and code for a protein, the molecular weight of which was estimated as 80.1 kDa. This molecular weight value almost equals the value obtained by SDS-PAGE analysis of the purified novel transferase derived from the Sulfolobus solfataricus strain ATCC 33909. Additionally, the existence of the activity of the novel transferase in a transformant containing the plasmid pO9T1 was confirmed according to the procedure described in Example I-14.

### EXAMPLE I-17

### Hybridization Tests Between the Gene Coding for the Novel Transferase Derived from the Sulfolobus solfataricus strain KM1 and Chromosome DNAs Derived from the Other Organisms

Chromosome DNAs were obtained from the Sulfolobus solfataricus strain DSM 5833, the Sulfolobus shibatae strain DSM 5389, and the E. coli strain JM109, and digested with restriction enzymes Pst I and Eco RI.

These digested products were separated by 1% agarose gel electrophoresis and transferred using the Southern blot technique to a Hybond-N membrane manufactured by Amersham Japan Co. The Sph I-Xba I fragment of about 2.6 kbp (corresponding to the sequence shown in Sequence No. 1, or corresponding to the region of A-B in FIG. 26), which derived from pKT21 obtained in Example I-12, was labeled using a DIG system kit manufactured by Boehringer Mannheim Co., and the resultant was subjected to a hybridization test with the above-prepared membrane.

The hybridization was performed under the conditions of 40° C. for 2 hours with 5×SSC, and washing was performed by treating twice with 2×SSC-containing 0.1% of SDS at 40° C. for 5 min., and twice with 0.1×SSC containing 0.1% of SDS at 40° C. for 5 min.

As a result, the Sph I-Xba I fragment could hybridize with a fragment of about 5.9. kbp derived from the Sulfolobus solfataricus strain DSM 5833, and with fragments, of about 5.0 kbp and about 0.8 kbp, respectively, derived from the Sulfolobus shibatae strain DSM 5389. On the other hand, no hybrid formation was observed in fragments derived from the E. coli strain JM109 which was used as a negative control.

Further, chromosome DNAs were obtained according to the procedure described in Example I-10 from the Sulfolobus solfataricus strains KM1, DSM 5354, DSM 5833, ATCC 35091, and ATCC 35092; the Sulfolobus acidocaldarius strains ATCC 33909, and ATCC 49426; the Sulfolobus shibatae strain DSM 5389; the Acidianus brierleyi strain DSM 1651; and the E. coli strain JM109, and digested with restriction enzymes, Hind II, Xba I, and Eco RV.

These digested products-were separated-by 1% agarose gel electrophoresis and transferred using the Southern blot technique to a Hybond-N+membrane manufactured by Amersham Japan Co. The region (378 bp) from the 1880th base to the 2257th base of Sequence No. 1 was amplified by PCR and labeled with ³²p according to the procedure described in Example I-16, and the resultant was subjected to a hybridization test with the above prepared membrane.

The hybridization was performed overnight under the conditions of 60° C. with 6×SSPE containing 0.5% of SDS, and washing was performed by treating twice with 2×SSPE containing 0.1% of SDS at room temperature for 10 min.

As a result, the following fragments were found to form hybrids: the fragments of about 4.4 kbp, about 3.7 kbp, about 3.7 kbp, about 0.8 kbp, and about 3.9 kbp derived from the Sulfolobus solfataricus strains KM1, DSM 5354, DSM 5833, ATCC 35091, and ATCC 35092, respectively; the fragments of about 0.8 kbp, and about 0.8 kbp derived from the Sulfolobus acidocaldarius strains ATCC 33909, and ATCC 49426, respectively; the fragment of about 4.4 kbp derived from the Sulfolobus shibatae strain DSM 5389; and the fragment of about 2.1 kbp derived from the Acidianus brierleyi strain DSM 1651. On the other hand, no hybrid formation was observed as to the genome DNA of the strain JM109.

Moreover, it was confirmed, through data banks of amino acid sequences (Swiss prot and NBRF-PDB) and a data bank of base sequences (EMBL) , and by using sequence-analyzing software, GENETYX (produced by Software Development Co.), that there is no sequence homologous to any of the amino acid sequences and base sequences within the scopes of Sequences No. 1, No. 2, No. 3, and No. 4. Consequently, the genes coding for the novel transferases were found to be highly conserved specifically in archaebacteria belonging to the order Sulfolobales.

### EXAMPLE I-18

### Comparisons Between the Base Sequences and Between the Amino Acid Sequences of the Novel Transferases Derived from the Sulfolobus solfataricus strain KM1 and the Sulfolubus acidocaidarius Strain ATCC 33909

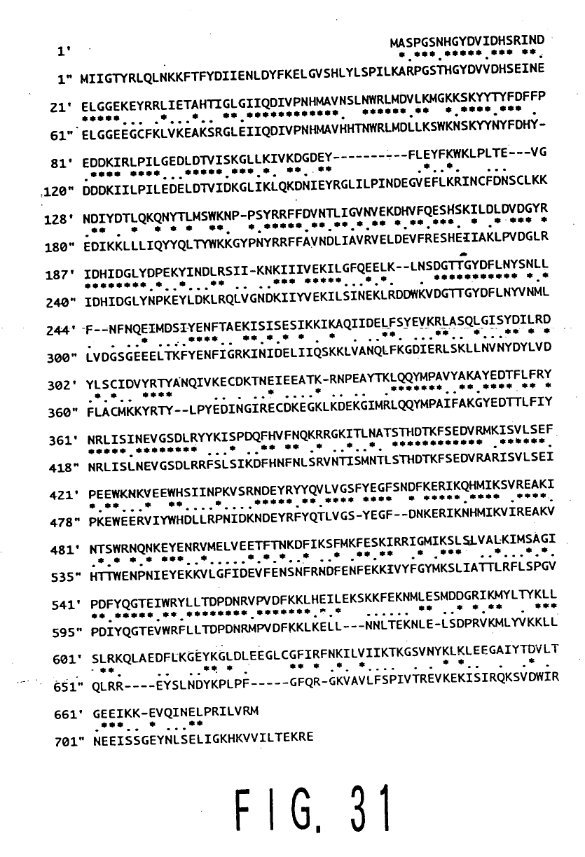

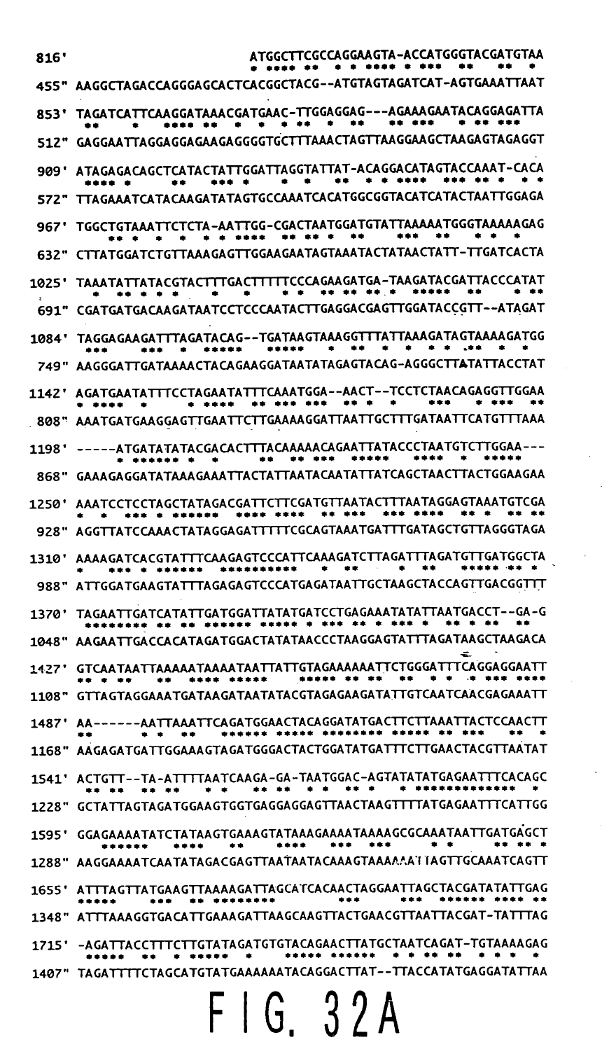

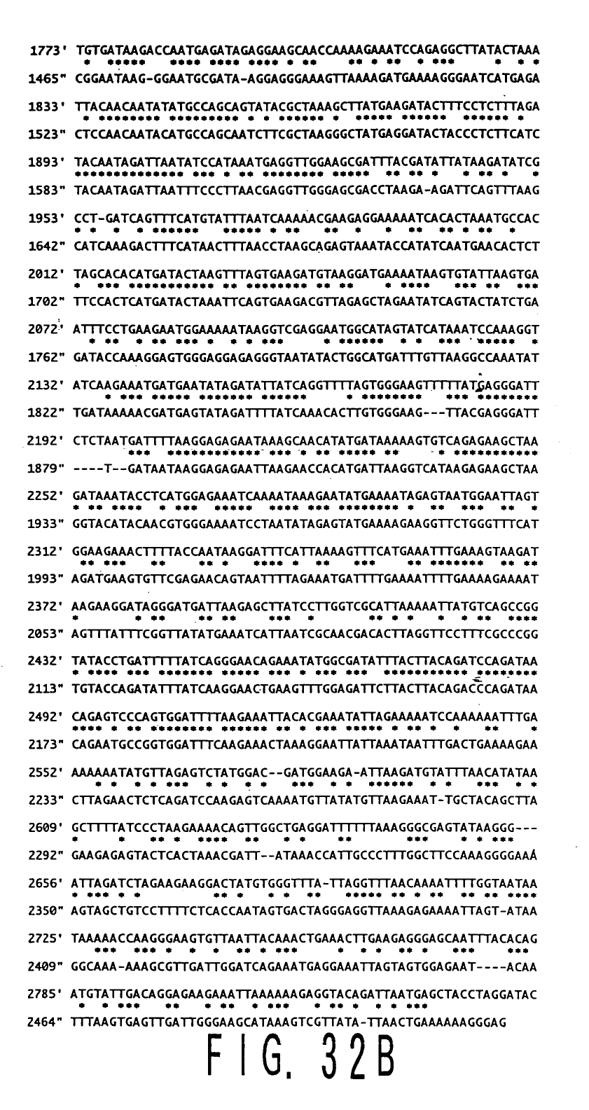

Considering gapps and using sequence-analyzing software, GENETYX (produced by Software Development Co.), comparative analyses-were carried out on the amino acid sequence of the novel transferase derived from the strain KM1, i.e. Sequence No. 2, and that derived from the strain ATCC 33909, i.e. Sequence No. 4; and on the base sequence coding for the novel transferase derived from the strain KM1, i.e. Sequence No. 1, and that derived from the strain ATCC 33909, i.e. Sequence No. 3. The results as to the amino acid sequences are shown in FIG. 31, and the results as to the base sequences are shown in FIG. 32. In each figure, the upper line indicates the sequence derived from the strain 33909, the lower-line indicates the sequence derived from the strain KM1, and the symbol “*” in the middle line indicates the portions equal in both strains. Each of the couples indicated with symbol “.” in FIG. 31 are a couple of amino acid residues which mutually have similar characteristics. The homology values are 49% and 57% on the levels of the amino acid sequences and the base sequences, respectively.

### EXAMPLE I-19

### Production of Trehaloseoligosaccharides from a Maltooligosaccharide Mixture Using the Recombinant Novel Transferase Derived from a Transformant

Alpha-amylase-hydrolysate obtained by hydrolyzing soluble starch (manufactured by Nacalai tesque Co., special grade) into oligosaccharides which do not cause the iodo-starch reaction was used as a substrate, wherein the α-amylase was A-0273 manufactured by Sigma Co. and derived from Aspergillus oryzae. Production of glucosyltrehalose and various maltooligosyltrehaloses was attempted by using the crude enzyme solution obtained in Example I-14 and the above substrate, and according to the reaction conditions described in Example I-14. The obtained reaction mixture was analyzed by a HPLC method under the following conditions.

| | |
|---|---|
| Column: | BIORAD AMINEX HPX-42A (7.8 × 300 mm) |
| Solvent: | Water |
| Flow rate: | 0.6 ml/min. |
| Temperature: | 85° C. |
| Detector: | Refractive Index Detector |

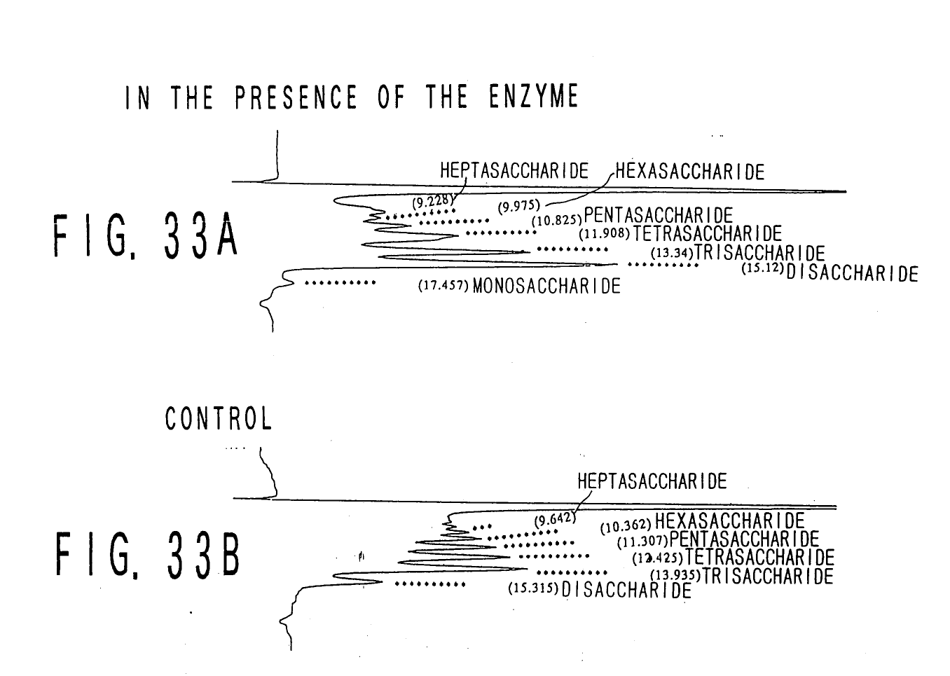

The results by HPLC analysis are shown in FIG. 33(A), and the results by HPLC analysis in a case performed without the recombinant novel transferase are shown in FIG. 33(B). As is obvious from the results, each of the oligosaccharides as the reaction products exhibits a retention time shorter than those of the reaction products produced in the: control group, namely, produced only with amylase. Next, the principal products, i.e. trisaccharide, tetrasaccharide, and pentasaccharides derived from the substrates, i.e. maltotriose (G3), maltotetraose (G4), and maltopentaose (G5) (all manufactured by Hayashibara Biochemical Co.), respectively, were isolated using the TSK-gel Amide-80 HPLC column, and were analyzed by ¹H-NMR and ¹³C-NMR. As a result, all of such products were found to have a structure in which the glucose residue at the reducing end is α-1,α-1-linked, and the products were confirmed as glucosyltrehalose (α-D-maltosyl α-D-glucopyranoside), maltosyltrehalose (α-D-maltotriosyl α-D-glucopyranoside), and maltotriosyltrehalose (α-D-malto-tetraosyl α-D-glucopyranoside), respectively.

### EXAMPLE I-20

### Production of Glucosyltrehalose and Maltooligosyltrehalose by Using the Novel Transferase Derived from a Transformant

Maltotriose (G3)-Maltoheptaose (G7) (all manufactured by Hayashibara Baiokemikaru Co.) were used as substrates. The crude enzyme solution obtained in Example I-14 was lyophilized, and then suspended in a 50 mM sodium acetate solution (pH 5.5) to make a concentrated enzyme solution. Each of the substrates was subjected to reaction with 12.7 Units/ml (in terms of the enzymatic activity when maltotriose is used as the substrate) of the concentrated enzyme solution to produce a corresponding α-1,α-1-transferred isomer. Each reaction product was analyzed by the method described in Example I-1 to examine the yield and the enzymatic activity. The results are shown in Table 38. Incidentally, as to the enzymatic activity shown in Table 38, 1 Unit is defined as an enzymatic activity of transferring maltooligosaccharide to produce 1 μmol per hour of a corresponding α-1,α-1-transferred isomer.

TABLE 38

| | Enzyme activity | Yield |
|---|---|---|
| Substrate | (unit/ml) | (%) |
| Maltotriose (G3) | 12.7 | 40.8 |
| Maltotetraose (G4) | 72.5 | 69.8 |
| Maltopentaose (G5) | 103.5 | 65.3 |
| Maltohexaose (G6) | 87.3 | 66.5 |
| Maltoheptaose (G7) | 60.2 | 67.9 |

### EXAMPLE II-15

### Determination of the Partial Amino Acid Sequences of the Novel Amylase Derived from the Sulfolobus solfataricus strain KM1

The partial amino acid sequences of the purified enzyme obtained in Example II-2 were determined by the method disclosed in Iwamatsu, et al. [Seikagaku (Biochemistry) 63, 139 (1991)], and the amino acid sequence of the N terminus side was determined by the method disclosed in Matsudaira, T. [J. Biol. Chem. 262, 10035-10038 (1987)].

At first, the purified novel amylase was suspended in a buffer solution for electrophoresis [10% glycerol, 2.5% SDS, 2% 2-mercaptoethanol, 62 mM Tris-Hcl buffer solution (pH 6.8)], and subjected to SDS-Polyacrylamide gel electrophoresis. After the electrophoresis, the enzyme was transferred from the gel to a polyvinylidene diflorido (PVDF) membrane (ProBlot, manufactured by Applied Biosystems Co.) by electroblotting (SartoBlot type IIs, manufactured by Sartorius Co.) with 160 mA for 1 hour.

After the transfer, the portion to which the enzyme had been transferred was cut out from the membrane, and soaked in about 300 μl of a buffer solution for reduction [6 M guanidine-HCl, 0.5 M Tris-HCl buffer solution (pH 3.5) containing 0.3% of EDTA and 2% of acetonitrile]. One milligram of dithiothreitol was added to this, and reduction was carried out under an argon atmosphere at 60° C. for 1 hour, approximately. To the resultant, 2.4 mg of monoiodoacetic acid dissolved in 10 μl of 0.5 N sodium hydroxide was added and stirred for 20 min. in the dark. The PVDF membrane was then taken out and washed sufficiently with a 2% acetonitrile solution, and subsequently, stirred in a 0.1% SDS solution for 5 min. After being briefly washed with water, the PVDF membrane was then soaked in a 100 mM acetic acid solution containing 0.5% of Polyvinylpyrrolidone-40, and was left standing for 30 min. Next, the PVDF membrane was briefly washed with water, and cut into pieces of 1 square mm, approximately. For determination of the amino acid sequence of the N terminus side, these pieces from the membrane were directly analyzed with a gas-phase sequencer. For determination of the partial amino acid sequences, these pieces were further soaked in a buffer solution for digestion [8% acetonitrile, 90 mM Tris-HCl buffer solution (pH 9.0)], and after the addition of 1 pmol of the Achromobacter Protease I (manufactured by Wako pure chemical Co.), digested at room temperature spending 15 hours. The digested products were separated by reversed phase chromatography using a C8 column (μ-Bondashere 5C8, 300A, 2.1×150 mm, manufactured by Millipore Ltd. Japan) to obtain a dozen or more kinds of peptide fragments. Using A solvent (0.05% trifluoroacetic acid) and B solvent (2-propanol:acetonitrile=7:3, containing 0.02% of trifluoroacetic acid) as elution solvents, the peptides were eluted with a linear concentration gradient from 2 to 50% relative to B solution and at a flow rate of 0.25 ml/min. for 40 min. As to the peptide fragments thus obtained, the amino acid sequences were determined by the automatic Edman degradation method using a gas-phase peptide sequencer (model 470, manufactured by Applied Biosystems Co.).

The amino acid sequence of the N terminus and the partial amino acid sequences thus determined are as follows.

Amino Acid Sequence of the N Terminus Side

| | |
|---|---|
| Thr Phe Ala Tyr Lys Ile Asp Gly | (Sequence No. 45) |
| Asn Glu | |

Partial Amino Acid Sequences

| | | |
|---|---|---|
| P-6: | Leu Gly Pro Tyr Phe Ser | (Sequence No. 46) |
| | Gln | |
| P-7: | Asp Val Phe Val Tyr Asp | (Sequence No. 47) |
| | Gly | |
| P-10: | Tyr Asn Arg Ile Val Ile | (Sequence No. 48) |
| | Ala Glu Ser Asp Leu Asn | |
| | Asp Pro Arg Val Val Asn | |
| | Pro | |

### EXAMPLE II-16

### Preparation of Chromosome DNA of the Sulfolobus solfataricus strain KM1

The Sulfolobus solfataricus strain KM1 was cultivated at 75° C. for 3 days in the culture medium which is identified as No. 1304 in Catalogue of Bacteria and Phages 18th edition (1992) published by American Type Culture Collection (ATCC), and which contained 2 g/liter of soluble starch and 2 g/liter of yeast extract. The cultivated bacteria was collected by centrifugation and stored at −80° C. The yield of the bacterial cell was 3.3 g/liter.

To 1 g of the bacterial bodies, 10 ml of a 50 mM Tris-HCl buffer solution (pH 8.0) containing 25% of sucrose, 1 mg/ml of lysozyme, 1 mM of EDTA, and 150 mM of NaCl was added for making a suspension, and the suspension was left standing for 30 min. To this suspension, 0.5 ml of 10% SDS and 0.2 ml of 10 mg/ml Proteinase K (manufactured by Wako pure chemical Co.) were added, and the mixture. was left standing at 37° C. for 2 hours. Next, the mixture was subjected to extraction with a phenol/chloroform solution, and then subjected to ethanol precipitation. The precipitated DNA was twisted around a sterilized glass stick and vacuum-dried after being washed with a 70% ethanol solution. As the final product, 1.5 mg of the chromosome DNA was obtained.

### EXAMPLE II-17

### Expression Cloning of a Gene Coding for the Novel Amylase Derived from the Sulfolobus solfataricus Strain KM1 by an Activity Staining Method

One hundred micrograms of the chromosome DNA of the Sulfolobus solfataricus strain KM1, prepared in Example II-16, was partially digested with a restriction enzyme, Sau 3AI. The reaction mixture was ultracentrifuged with a density gradient of sucrose to isolate and purify DNA fragments of 5-10 kb. Then, using T4 DNA ligase, the above chromosome DNA fragments having lengths of 5-10 kb were ligated with a modified vector which had been prepared from a plasmid vector, pUC118 (manufactured by Takara Shuzou Co.), by digestion with Bam HI and by dephosphorylation of the ends with alkaline phosphatase. Next, cells of the E. coli strain JM109 (manufactured by Takara Shuzou Co.) were transformed with a mixture containing the modified pUC118 plasmid vectors in which any of the fragments had been inserted. These cells were cultivated on LB agar plates containing 50 μg/ml of ampicillin to grow their colonies and make a DNA library.

Screening of the transformants which have a recombinant plasmid containing a gene coding for the novel amylase derived from the Sulfolobus solfataricus strain KM1 was performed by an activity staining method.

At first, the obtained transformants were replicated on filter paper and cultivated on an LB agar plate for colonization. The filter paper was dipped in a 50 mM Tris-HCl buffer solution (pH 7.5.) containing 1 mg/ml of lysozyme (manufactured by Seikagaku Kougyou Co.) and 1 mM of EDTA, and was left standing for 30 min. Subsequently, the filter paper was dipped in 1% Triton-X100 solution for 30 min. for bacteriolysis, and heat-treated at 60° C. for 1 hour to inactivate the enzymes derived from the host. The filter paper thus treated was then laid on an agar plate containing 0.2% of soluble starch to progress a reaction at 60° C., overnight. The plate subjected to the reaction was put under the iodine-vapor atmosphere to make the starch get color. The colonies which exhibit a halo was recognized as the colonies of positive clones. As a result, five positive clones were obtained from 6,000 transformants. According to analysis of the plasmids extracted from these clones, an insertional fragment of about 4.3 kbp was contained in a plasmid as the shortest insertional fragment.

Further, the insertional fragment was shortened by subjecting it to digestion with Bam HI and the same procedure as above. As a result, a transformant containing a plasmid which has an insertional fragment of about 3.5 kb was obtained. This plasmid was named as pKA1.

The restriction map of the insertional fragment of this plasmid is shown in FIG. 34.

### EXAMPLE II-18

### Determination of the Gene Coding for the Novel Amylase Derived from the Sulfolobus solfataricus strain KM1

The base sequence of the insertional fragment in the plasmid, pKA1 obtained in Example II-17, (i.e. the DNA of the region corresponding to the plasmid, pKA2, described below) was determined.

At first, a deletion plasmid was prepared from the above plasmid DNA by using a deletion kit for kilo-sequencing which was manufactured by Takara Shuzou Co. After that, the DNA sequence of the insertional fragment in the plasmid were determined by using a sequenase dye primer sequencing kit, PRISM, a terminator cycle sequencing kit, Tag Dye Deoxy™, both manufactured by Perkin Elmer Japan Co., and a DNA sequencer, GENESCAN Model 373A, manufactured by Applied Biosystems Co.

The base sequence, and the amino sequence anticipated therefrom are shown in Sequences No. 5 and No. 6, respectively.

Sequences corresponding to any of the partial amino acid sequences obtained in Example II-15, respectively, were recognized in the above amino acid sequence. This amino acid sequence was assumed to have 558 amino acid residues and code for a protein, the molecular weight of which estimated as 64.4 kDa. This molecular weight value almost equals the value, 61.0 kDa, obtained by SDS-PAGE analysis of the purified novel amylase derived from the Sulfolobus solfataricus strain KM1.

### EXAMPLE II-19

### Production of the Recombinant Novel Amylase in a Transformant

A plasmid, pKA2, was obtained by partially digesting the plasmid, pKA1, which was obtained in Example II-17, with a restriction enzyme, Pst I. FIG. 35 shows its restriction map. The enzymatic activity of the transformant which contains pKA2 was examined as follows. At first, the above transformant was cultivated overnight in a LB broth containing 100 μg/ml of ampicillin at 37° C. The cells collected by centrifugation were suspended in 4 ml/g-cell of a 50 mM sodium acetate solution (pH 5.5), and subjected to ultrasonic crushing-treatment and centrifugation. The supernatant thus obtained was heat-treated at 70° C. for 1 hour to inactivate the amylase derived from the host cells. The precipitate was removed by centrifugation and the resultant was concentrated with an ultrafiltration membrane (critical molecular weight: 13,000) to obtain a crude enzyme solution which would be used in the following experiments.

(1) Substrate Specificity

The hydrolyzing properties and the hydrolyzed products were analyzed by allowing 35.2 Units/ml of the above crude enzyme solution to act on the various 10 mM substrates (except amylopectin and soluble starch were used as 3.0% solutions) listed in Table 39 below. Here, 1 Unit was defined as an enzymatic activity of producing 1 μmol of α,α-trehalose per hour from maltotriosyltrehalose used as the substrate under the conditions based on those in Example II-1. The analysis was performed by TSK-gel Amide-80 HPLC described in Example II-1, wherein the index was the activity of producing both monosaccharide and disaccharide when the substrate was each of the various maltooligosaccharides, Amylose DP-17, amylopectin, soluble starch, various isomaltooligosaccharides, and panose; the activity of producing α,α-trehalose when the substrate was each of the various trehaloseoligosaccharides, and α-1,α-1-transferred isomer of Amylose DP-17 (the oligosaccharide derived from Amylose DP-17 by transferring the linkage between the first and second glucose residues from the reducing end side into an α-1,α-1 linkage); and the activity of producing glucose when the substrate was maltose or α,α-trehalose.

The results are as shown in Table 39 below.

Incidentally, each enzymatic activity value in the table is expressed with such a unit as 1 Unit equals the activity of liberating 1 μmol of each of the monosaccharide and disaccharide per hour.

TABLE 39

| | | Production rate of |
|---|---|---|
| | | mono- and |
| | Liberated | disaccharides |
| Substrate | oligosaccharide | (units/ml) |
| Maltose (G2) | Glucose | 0.15 |
| Maltotriose (G3) | Glucose + G2 | 0.27 |
| Maltotetraose (G4) | Glucose + G2 + G3 | 0.26 |
| Maltopentaose (G5) | Glucose + G2 + G3 + G4 | 2.12 |
| Amylose DP-17 | Glucose + G2 | 2.45 |
| Amylopectin | Glucose + G2 | 0.20 |
| Soluble starch | Glucose + G2 | 0.35 |
| α,α-Trehalose | not decomposed | 0 |
| Glucosyltrehalose | Glucose + Trehalose | 0.01 |
| Maltosyltrehalose | G2 + Trehalose | 4.52 |
| Maltotriosyltrehalose | G3 + Trehalose | 35.21 |
| Amylose DP-17, α-1, | Trehalose | 4.92 |
| α-1-transferred isomer | | |
| Isomaltose | not decomposed | 0 |
| Isomaltotriose | not decomposed | 0 |
| Isomaltotetraose | not decomposed | 0 |
| Isomaltopentaose | not decomposed | 0 |
| Panose | not decomposed | 0 |

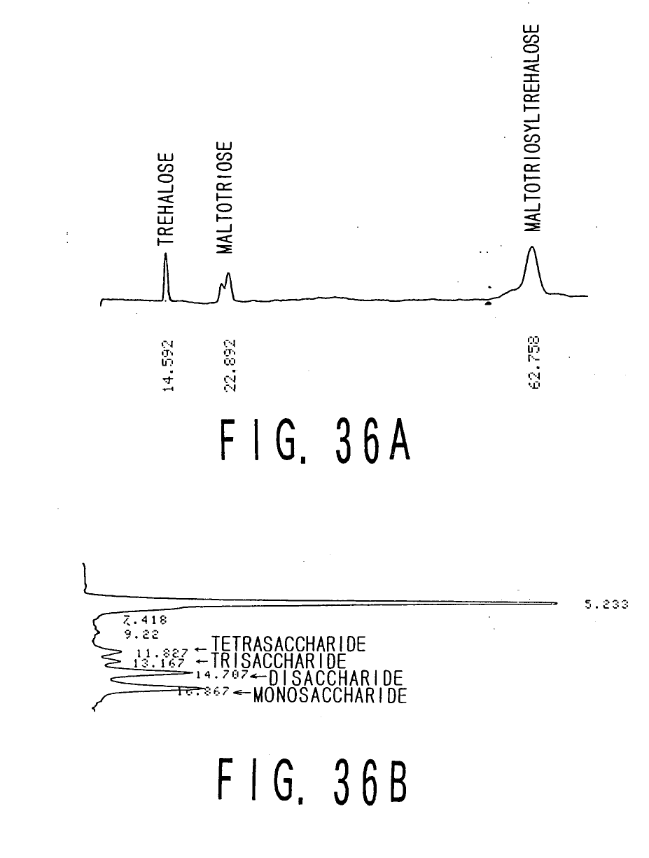

Further, the analytic results of the reaction products from maltotriosyltrehalose by TSK-gel Amide-80 HPLC under the conditions based on those in Example II-1 are shown in FIG. 36(A). Moreover, the analytic results of the reaction products from soluble starch by AMINEX HPX-42A HPLC under the conditions described below are shown in FIG. 36(B).

| | |
|---|---|
| Column: | AMINEX HPX-42A (7.8 × 300 mm) |
| Solvent: | Water |
| Flow rate: | 0.6 ml/min. |
| Temperature: | 85° C. |
| Detector: | Refractive Index Detector |

From the above results, the present enzyme was confirmed to markedly effectively act on a trehaloseoligo-saccharide, of which the glucose residue at the reducing end is α-1,α-1-linked, such as maltotoriosyltrehalose, to liberate α,α-trehalose and a corresponding maltooligosac-charide which has a polymerization degree reduced by two. Further, the present enzyme was confirmed to liberate principally glucose or maltose from maltose (G2)-maltopentaose (G5), amylose, and soluble starch. The present enzyme, however, did not act on α,α-trehalose, isomaltose, isomaltotriose, isomaltotetraose and isomaltopentaose, and panose.

(2) Endotype Amylase Activity

One hundred and fifty Units/ml [in terms of the same unit as that in the above (1)] of the above crude enzyme solution was allowed to act on soluble starch. The time-lapse change in the degree of coloring by the iodo-starch reaction was measured under the same conditions as the method for measuring starch-hydrolyzing activity in Example II-1. Further, produced amounts of monosaccharide and disaccharide were measured under the conditions based on those in the HPLC analysis method which is described in the above (1), namely, based on those for the above examination of substrate specificity. From the data thus obtained, a starch-hydrolyzing rate was estimated.

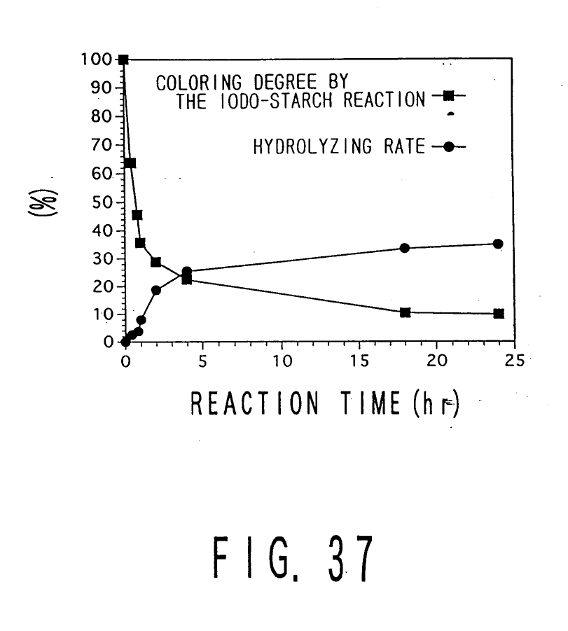

The time-lapse change is shown in FIG. 37. As shown in the figure, the hydrolyzing rate at the point where the coloring degree by the iodo-starch reaction decreased to 50% was as low as 4.5%. Accordingly, the present crude enzyme was confirmed to have a property of an endotype amylase.

(3) Investigation of the Action Mechanism

Uridinediphosphoglucose [glucose-6-³H] and malto-tetraose were put into a reaction with glycogen synthase (derived from rabbit skeletal muscle, G-2259 manufactured by Sigma Co.) to synthesize maltopentaose, of which the glucose residue of the non-reducing end was radiolabeled with ³H, and the maltopentaose was isolated and purified. To 10 mM of this maltopentaose radiolabeled with ³H as a substrate, 10 Units/ml (in terms of the unit used in Example I-1) of the recombinant novel transferase obtained in Example I-20 above was added and put into a reaction at 60° C. for 3 hours. Maltotriosyltrehalose, of which the glucose residue of the non-reducing end was radiolabeled with ³H, was synthesized thereby, and the product was isolated and purified. Incidentally, it was confirmed by the following procedure that the glucose residue of the non-reducing end had been radiolabeled: The above product was completely decomposed into glucose and α,α-trehalose by glucoamylase (derived from Rhizopus, manufactured by Seikagaku Kougyou Co.); the resultants were sampled by thin-layer chromatography, and their radioactivities were measured by a liquid scintillation counter; as a result, radioactivity was not observed in the α,α-trehalose fraction but in the glucose fraction.

The above-prepared maltopentaose and maltotriosyl-trehalose, of which the glucose residues of the non-reducing ends were radiolabeled with ³H, were used as substrates, and were put into reactions with 30 Units/ml and 10 Units/ml of the above crude enzyme solution, respectively. Sampling was performed before the reaction and 3 hours after the start of the reaction performed at 60° C. The reaction products were subjected to development by thin-layer chromatography (Kieselgel 60 manufactured by Merk Co.; solvent: butanol/ethanol/water=5/5/3). Each spot thus obtained and corresponding to each saccharide was collected, and its radiation was measured with a liquid scintillation counter. When maltopentaose was used as a substrate, radioactivity was not detected in the fractions of the hydrolysates, i.e. glucose and maltose, but in the fractions of maltotetraose and maltotriose. On the other hand, when maltotriosyltrehalose was used as a substrate, radioactivity was not detected in the fraction of the hydrolysate, i.e. α,α-trehalose, but in the fraction of maltotriose.

Consequently, as to the action mechanism, the recombinant novel amylase was found to have an amylase activity of the endotype function, and in addition, an activity of principally producing monosaccharide and disaccharide from the reducing end side.

Incidentally, the manufacturer of the reagents used in the above experiments are as follows.

α,α-trehalose: Sigma Co.

Maltose (G2): Wako Junyaku Co.

Maltotriose-Maltopentaose (G3-G5): Hayashibara Baiokemikaru Co.

Amylose DP-17: Hayashibara Biochemical Co.

Isomaltose: Wako pure chemical Co.

Isomaltotriose: Wako pure chemical Co.

Isomaltotetraose: Seikagaku Kougyou Co.

Isomaltopentaose: Seikagaku Kougyou Co.

Panose: Tokyo Kasei Kougyou Co.

Amylopectin: Nacalai tesque Co.

### EXAMPLE II-20

### Determination of Partial Amino Acid Sequences of the Novel Amylase Derived from the Sulfolobus acidocaldarius strain ATCC 33909

The partial amino acid sequences of the purified enzyme obtained in Example II-4 were determined according to the process described in Example II-15.

The partial amino acid sequences are as follows.

| | | |
|---|---|---|
| AP-9: | Leu Asp Tyr Leu Lys | (Sequence No. 49) |
| AP-10: | Lys Arg Glu Ile Pro Asp | (Sequence No. 50) |
| | Pro Ala Ser Arg Tyr Gln | |
| | Pro Leu Gly Val His | |
| AP-11: | Lys Asp Val Phe Val Tyr | (Sequence No. 51) |
| | Asp Gly Lys | |

### EXAMPLE II-21

### Preparation of DNA Probes Based on the Partial Amino Acid Sequences of the Novel Amylase Derived from the Sulfolobus acidocaldarius strain ATCC 33909

According to information about the partial amino acid sequences determined in Example II-20, oligonucleotide DNA primers are prepared by using a DNA synthesizer (Model 381 manufactured by Applied Biosystems Co.). Their sequence were as follows.

AP-10

Amino Acid Sequence

| | | |
|---|---|---|
| N terminus | Pro Ala Ser Arg Tyr Gln Pro | C terminus |
| DNA Primer | 5′ AGCTAGTAGATATCAACC 3′ | (Sequence No. 57) |
| Base Sequence | A G C C G | |

AP-11

(Complementary Strand)

Amino Acid Sequence

| | | |
|---|---|---|
| N terminus | Asp Val Phe Val Tyr Asp Gly Lys | C terminus |
| DNA Primer | 5′ TTTTCCATCATAAACAAAAACATC 3′ | (Sequence No. 58) |
| Base Sequence | C A G T G T | |
| | C | |

PCR was performed using 100 pmol of each primer and about 100 ng of the chromosome DNA prepared in Example II-16 and derived from the Sulfolobus acidocaldarius strain ATCC 33909. The PCR apparatus used herein was Gene Amp PCR system Model 9600, manufactured by Perkin Elmer Co. In the reaction, 30 cycles of steps were carried out with 100 μl of the total reaction mixture, wherein the 1 cycle was composed of steps at 94° C. for 30 sec., at 54° C. for 30 sec., and at 72° C. for 30 sec. The amplified fragment of about 830 bp was subcloned into a plasmid, pT7 Blue T-Vector (manufactured by Novagen Co.). Determination of the base sequence of the insertional fragment in this plasmid was performed to find sequences corresponding to any of the amino acid sequences obtained in Example II-20.

### EXAMPLE II-22

### Cloning of a Gene Coding for the Novel Amylase Derived from the Sulfolobus acidocaldarius Strain ATCC 33909

The chromosome DNA of the Sulfolobus acidocaldarius strain ATCC 33909 was obtained according to the process described in Example II-16 from bacterial cells obtained according to the process described in Example II-4. The above chromosome DNA was partially digested with Sau 3AI, and subsequently, ligated to a Bam HI-restricted arm of EMBL3 (manufactured by STRATAGENE Co.) by using T4 DNA ligase. Packaging was carried out using Gigapack II Gold, manufactured by STRATAGENE Co. With the library obtained above, the E. coli strain LE392 was infected at 37° C. for 15 min., inoculated on NZY agar plates, and incubated at 37° C. for 8-12 hours, approximately, to form plaques. After being stored at 4° C. for about 2 hours, DNA was adsorbed on a nylon membrane (Hybond N+, manufactured by Amersham Co. Baking was performed at 80° C. for 2 hours after brief washing with 2×SSPE. Using the PCR fragment obtained in Example II-21, the probe was labeled with ³²p employing Megaprime DNA labeling system manufactured by Amersham Co.

Hybridization was performed overnight under the conditions of 65° C. with 6×SSPE containing 0.5% of SDS. Washing was performed by treating twice with 2×SSPE containing 0.1% of SDS at room temperature for 10 min.

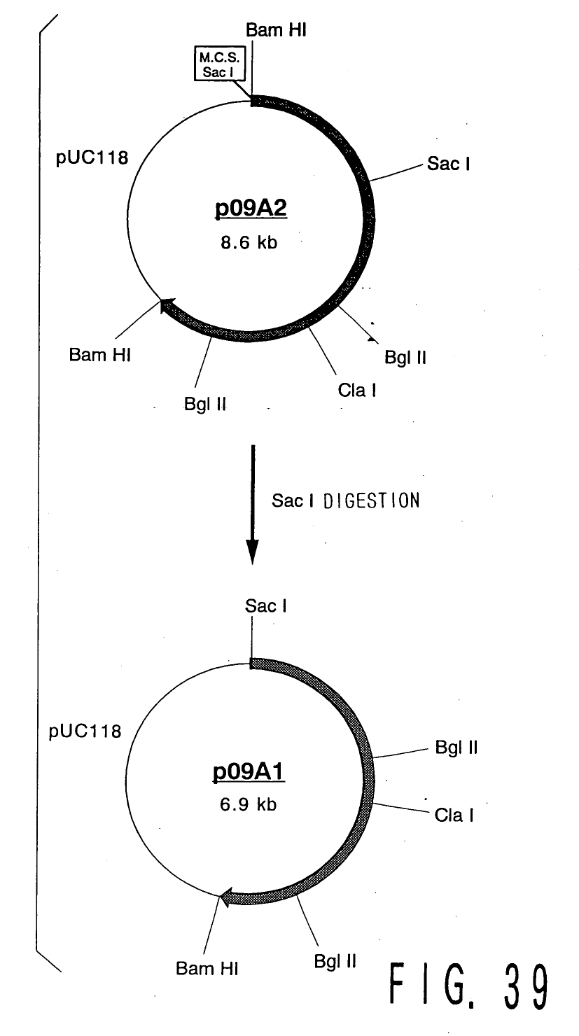

Screening was started with 8,000 clones, approximately, and 17 positive clones were obtained. From these clones, a Bam HI fragment of about 5.4 kbp was obtained and the fragment was inserted into pUC118 at the corresponding restriction site. The plasmid thus obtained was named as pO9A2. Further, the DNA of this plasmid was digested with Sau 3AI to obtain a plasmid named as pO9A1. The restriction map of the insertional fragment in pO9A1 is shown in FIG. 38, and the procedure for preparing pO9A1 is shown in FIG. 39. As to the above plasmid, pO9A1, a deletion plasmid was prepared using Double-standard Nested Delation Kit manufactured by Pharmacia Co. The base sequence, principally of the region corresponding to the structural gene of the novel amylase, was determined according to the process described in Example II-18. The base sequence thus determined and the amino acid sequence anticipated therefrom are shown in Sequences. No. 7 and No. 8, respectively. Sequences corresponding to any of the partial amino acid. sequences obtained in Example II-20, respectively, were recognized in this amino acid sequence. This amino acid sequence was assumed to have 556 amino acid residues and code for a protein, the molecular weight of which was estimated as 64.4 kDa. This molecular weight value almost equals the value obtained by SDS-PAGE analysis of the purified novel amylase derived from the Sulfolobus solfataricus strain ATCC 33909. Additionally, the existence of the activity of the novel amylase in a transformant containing the plasmid, pO9A1 was confirmed according to the procedure described in Example II-19.

### EXAMPLE II-23

### Homology Between the Base Sequences and Between the Amino Acid Sequences of the Novel Amylases Derived from the strain KM1 and the-strain ATCC 33909

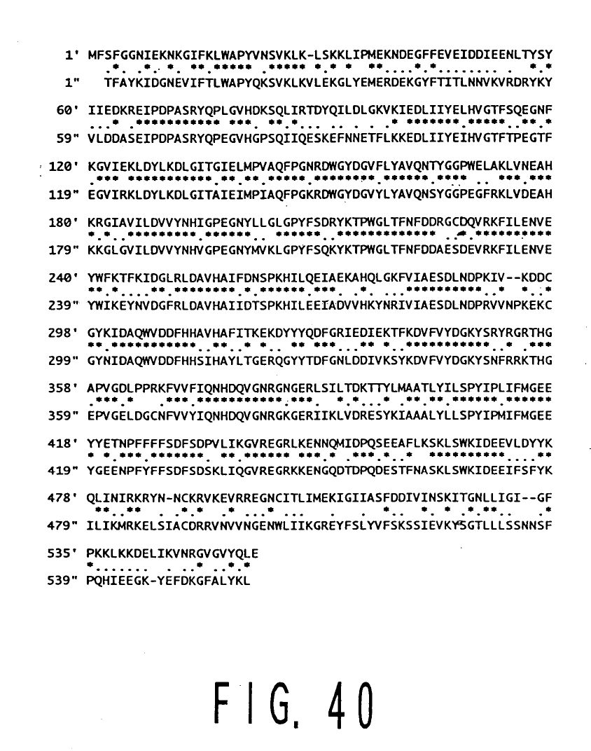

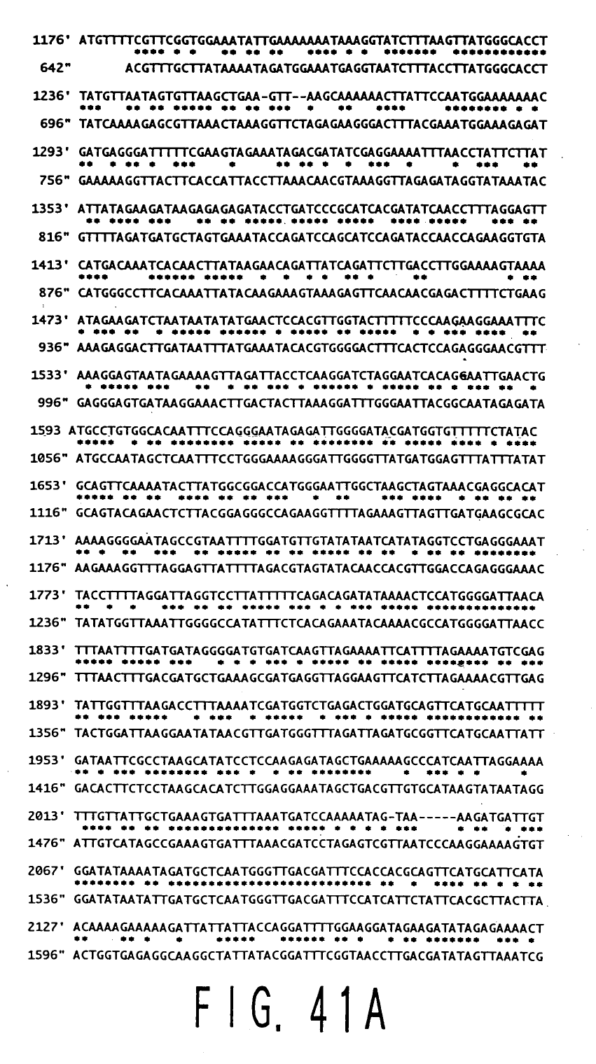

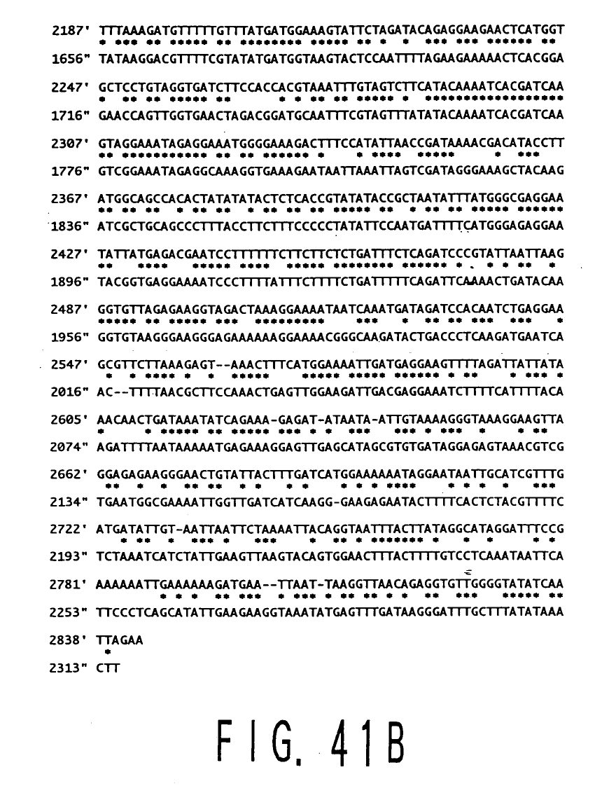

Considering gapps and using sequence-analyzing software, GENETYX (produced by Software Development Co.), comparative analyses were carried out on the amino acid sequence of the novel amylase derived from the strain KM1, i.e. Sequence No. 6, and that derived .from the strain ATCC 33909, i.e. Sequence No. 8; and on the base sequence coding for the novel amylase derived from the strain KM1, i.e. Sequence No. 5, and that derived from the strain ATCC 33909, i.e. Sequence No. 7. The results as to the amino acid sequences are shown in FIG. 40, and the results as to the base sequences are shown in FIG. 41. In each figure, the upper line indicates the sequence derived from the strain 33909, the lower line indicates the sequence derived from the strain KM1, and the symbol “*” in the middle line indicates the portions equal in both strains. Each of the couples indicated with symbol “.” in FIG. 40 are a couple of amino acid residues which mutually have similar characteristics. The homology values are about 59% and 64% on the levels of the amino acid sequences and the base sequences, respectively.

### EXAMPLE II-24

### Hybridization Tests between the Gene Coding for the Novel Amylase Derived from the Sulfolobus solfataricus strain KM1 or the Sulfolobus acidocaldarius Strain ATCC 33909 and Chromosome DNAs Derived from the Other Organisms

Chromosome DNAs were obtained from the Sulfolobus solfataricus strain DSM 5833, the Sulfolobus shibatae strain DSM 5389,, the Acidianus brierleyi strain DSM 1651, and the E. coli strain JM109, and digested with a restriction enzyme Hind III according to the procedure described in Example II-16.

These digested products were separated by 1% agarose gel electrophoresis, and transferred using the Southern blot technique to a Hybond-N membrane manufactured by Amersham Japan Co. The Pst I fragment of about 1.9 kbp (corresponding to the sequence from the 1st base to 1845th base of Sequence No. 5), which derived from pKA1 was labeled using a DIG system kit manufactured by Boehringer Mannheim Co., and the resultant was subjected to a hybridization test with the above-prepared membrane.

The hybridization was performed under the conditions of 40° C. for 3 hours with 5×SSC, and washing was performed by treating twice with 2×SSC containing 0.1% of SDS at 40° C. for 5 min., and twice with 0.1×SSC containing 0.1% of SDS at 40° C. for 5 min.

As a result, the Pst I fragment could hybridize with a fragment of about 13.0 kbp derived from the Sulfolobus solfataricus strain DSM 5833, a fragment of about 9.8 kbp derived from the Sulfolobus shibatae strain DSM 5389, and a fragment of about 1.9 kbp derived from the Acidianus brierleyi strain DSM 1651. On the other hand, no hybrid formation was observed in fragments derived from the E. coli strain JM109 which was used as a negative control.

Further, chromosome DNAs were obtained according to the procedure described in Example II-16 from the Sulfolobus solfataricus strains KM1, DSM 5354, DSM 5833, ATCC 35091, and ATCC 35092; the Sulfolobus acidocaldarius strains ATCC 33909, and ATCC 49426; the Sulfolobus shibatae strain DSM 5389; the Acidianus brierleyi strain DSM 1651; and the E. coli strain JM109, and digested with restriction enzymes, Xba I, Hind III, and Ecb RV. These digested products were separated by 1% agarose gel electrophoresis and transferred using the Southern blot technique to a Hybond-N+membrane manufactured by Amersham Japan Co. The region from the 1393th base to the 2121th base of Sequence No. 7 (obtained by digesting pO9A1 prepared in Example II-22 with restriction enzymes Eco T22I and Eco RV followed by separation in a gel) was labeled with ³²P according to the procedure described in Example II-22 to make a probe, and this probe was subjected to a hybridization test with the above prepared membrane. The hybridization was performed overnight under the conditions of 60° C. with 6×SSPE containing 0.5% of SDS, and washing was performed by treating twice with 2×SSPE containing 0.1% of SDS at room temperature for 10 min. As a result, the following fragments were found to form hybrids: the fragments of about 3.6 kbp, about 1.0 kbp, about 0.9 kbp, about 0.9 kbp, and about 1.0 kbp derived from the Sulfolobus solfataricus strains KM1, DSM 5354, DSM 5833, ATCC 35091, and ATCC 35092, respectively; the fragments of about 0.9 kbp, and about 0.9 kbp derived from the Sulfolobus acidocaldarius strains ATCC 33909, and ATCC 49426, respectively; the fragment of about 1.4 kbp derived from the Sulfolobus shibatae strain DSM 5389; and the fragment of about 0.9 kbp derived from the Acidianus brierleyi strain DSM 1651. On the other hand, no hybrid formation was observed as to the chromosome DNA of the E. coli strain JM109. Moreover, it was confirmed, through data banks of amino acid sequences (Swiss prot and NBRF-PDB) and a data bank of base sequences (EMBL), and by using sequence-analyzing software, GENETYX (produced by Software Development Co.), that there is no sequence homologous to any of the amino acid sequences and base sequences within the scopes of Sequences No. 5, No. 6, No. 7, and No. 8. Consequently, the genes coding for the novel amylases were found to be highly conserved specifically in archaebacteria belonging to the order Sulfolobales.

### EXAMPLE III-1

### Production of α,α-Trehalose by Using the Recombinant Novel Amylase and the Recombinant Novel Transferase

Production of α,α-trehalose was attempted by using the crude recombinant novel amylase obtained in Example II-19, the concentrated recombinant novel transferase obtained in Example I-20, and 10% soluble starch (manufactured by Nacalai tesque Co., special grade); and by supplementally adding pullulanase. The reaction was performed as follows.

At first, 10% soluble starch was treated with 0.5-50 Units/ml of pullulanase (derived from Klebsiella pneumoniae, and manufactured by Wako pure chemical Co.) at 40° C. for 1 hour. To the resultant, the above-mentioned recombinant novel transferase (10 Units/ml) and the above-mentioned recombinant novel amylase (150 Units/ml) were added, and the mixture was subjected to a reaction at pH 5.5 and 60° C. for 100 hours. The reaction was stopped by heat-treatment at 100° C. for 5 min., and the non-reacted substrate was hydrolyzed with glucoamylase. The reaction mixture was analyzed by an HPLC analyzing method under the conditions described in Example II-1.

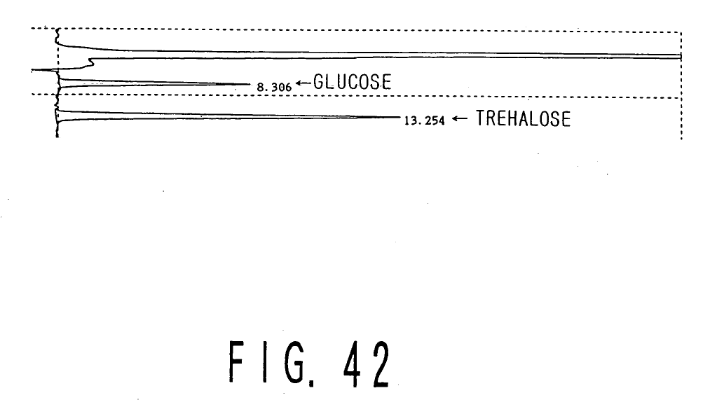

The analysis results by TSK-gel Amide-80 HPLC are shown in FIG. 42.

Here, as to enzymatic activity of the recombinant novel amylase, 1 Unit is defined as the activity of liberating 1 μmol of α,α-trehalose per hour from maltotriosyltrehalose. As to enzymatic activity of the recombinant novel transferase, 1 Unit is defined as the activity of producing 1 μmol of glucosyltrehalose per hour from maltotriose. As to enzymatic activity of pullulanase, 1 Unit is defined as the activity of producing 1 μmol of maltotriose per minute at pH 6.0 and 30° C. from pullulan.

The yield of α,α-trehalose was 67% when 50 Units/ml of pullulanase was added. This value suggests that the recombinant novel amylase can bring about almost the same yield as the purified novel amylase derived from the Sulfolobus solfataricus strain KM1 can under the above reaction condition.

### Industrial Applicability

A novel, efficient and high-yield process for producing trehaloseoligosaccharide, such as glucosyltrehalose and maltooligosaccharide, and other saccharides from a raw material such as maltooligosaccharide can be provided by using a novel transferase which is obtained by an enzyme-producing process according to the novel purification process of the present invention, and which can act on saccharides, such as maltooligosaccharide, to produce trehaloseoligosaccharide, such as glucosyltrehalose and maltooligosyltrehalose, and other saccharides.

A novel, efficient and high-yield process for producing α,α-trehalose from a glucide raw material such as starch, starch hydrolysate and maltooligosaccharide can be provided by using the novel amylase of the present invention in combination with the novel transferase of the present invention.

1. A novel transferase which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage.

2. A novel transferase which acts on a maltooligosaccharide, all the glucose residues of the maltooligosaccharide being α-1,4-linked, so as to transfer the first α-1,4 linkage from the reducing end into an α-1,α-1 linkage.

3. The novel transferase claimed in claim 1 or 2, wherein its molecular weight measured by SDS-polyacrylamide gel electrophoresis is 74,000 to 76,000, approximately.

4. The novel transferase claimed in any one of claims 1 to 3, wherein the transferase has the following physical and chemical properties:
(1) Optimum pH with in the range from 4.5 to 6.0;
(2) Optimum temperature within the range from 60 to 80° C.;
(3) ph Stability within the range from 4.5 to 10.0; and
(4) Thermostability which allow 90% or more of enzymatic activity to remain even after exposure at 80° C. for 6 hours:

5. The novel transferase claimed in any one of claims 1 to 4, wherein the isoelectric point measured by isoelectric focusing is pH 5.3 to pH 6.3.

6. The novel transferase claimed in any one of claims 1 to 5, wherein its activity can be fully inhibited with 5 mM CuSO₄.

7. The novel transferase claimed in any one of claims 1 to 6, wherein the transferase is derived from an archaebacterium belonging to the order Sulfolobales.

8. The novel transferase claimed in claim 7, wherein the transferase is derived from an archaebacterium belonging to the genus Sulfolobus.

9. The novel transferase claimed in claim 7, wherein the transferase is derived from an archaebacterium belonging to the genus Acidianus.

10. The novel transferase claimed in claim 8, wherein the archaebacterium belonging to the genus Sulfolobus is the Sulfolobus solfataricus strain KM1 (FERM BP-4626).

11. The novel transferase claimed in claim 8, wherein the archaebacterium belonging to the genus Sulfolobus is the Sulfolobus solfataricus strain DSM 5833.

12. The novel transferase claimed in claim 8, wherein the archaebacterium belonging to the genus Sulfolobus is the Sulfolobus acidocaldarius strain ATCC 33909.

13. The novel transferase claimed in claim 9, wherein the archaebacterium belonging to the genus Acidianus is the Acidianus brierleyi strain DSM 1651.

14. A process for producing the transferase which is claimed in any one of claims 1 to 13, wherein said process comprises cultivating a bacterium having an ability of producing the transferase claimed in any one of claims 1 to 13 in a culture medium, and isolating and purifying said transferase from the culture according to an activity-measuring method in which the index is the activity of producing a trehaloseoligosaccharide from a substrate maltooligosaccharide.

15. The process claimed in claim 14, wherein an archaebacterium belonging to the order Sulfolobales is cultivated.

16. The process claimed in claim 15, wherein an archaebacterium belonging to the genus Sulfolobus is cultivated.

17. The process claimed in claim 15, wherein an archaebacterium belonging to the genus Acidianus is cultivated.

18. The process claimed in claim 16, wherein the Sulfolobus solfataricus strain KM1 (FERM BP-4626) belonging to the genus Sulfolobus is cultivated.

19. The process claimed in claim 16, wherein the Sulfolobus solfataricus strain DSM 5833 belonging to the genus Sulfolobus is cultivated.

20. The process claimed in claim 16, wherein the Sulfolobus acidocaldarius strain ATCC 33909 belonging to the genus Sulfolobus is cultivated.

21. The process claimed in claim 17, wherein the Acidianus brierleyi strain DSM 1651 belonging to the genus Acidianus is cultivated.

22. A process for producing a saccharide, a couple of sugar units at an end of the saccharide being α-1,α-1-linked, wherein the transferase claimed in any one of claims 1 to 13 is used and allowed to act on a substrate saccharide, the substrate saccharide being composed of at least three sugar units wherein at least three glucose residues from the reducing end are α-1,4-linked, so as to produce a saccharide in which at least three sugar units from the reducing end side are glucose residues and the linkage between the first and second glucose residues from the reducing end side is α-1,α-1 while the linkage between the second and third glucose residues from the reducing end side is α-1,4.

23. The process claimed in claim 22, wherein the substrate is each or a mixture of maltooligosaccharides.

24. The process claimed in claim 23, wherein a trehaloseoligosaccharide such as glucosyltrehalose and maltooligosyltrehalose is produced.

25. A novel amylase which acts on a substrate saccharide, the substrate saccharide being composed of at least three sugar units wherein at least three sugar units from the reducing end are glucose residues, so as to liberate principally monosaccharides and/or disaccharides by hydrolyzing the substrate saccharide from the reducing end side.

26. The novel amylase claimed in claim 25 which has a principal activity of acting on a substrate saccharide, the substrate saccharide being composed of at least three sugar units wherein at least three sugar units from the reducing end side are glucose residues and the linkage between the first and the second glucose residues from the reducing end side is α-1,α-1 while the linkage between the second and the third glucose residues from the reducing end side is α-1,4, so as to liberate α,α-trehalose by hydrolyzing the α-1,4 linkage between the second and the third glucose residues.

27. The novel amylase claimed in claim 25 or 26, wherein said amylase also has an activity of endotype-hydrolyzing one or more α-1,4 linkages within the molecular chain of a substrate.

28. The novel amylase claimed in claim 25, 26 or 27, wherein said amylase has an activity of hydrolyzing a substrate trehaloseoligosaccharide such as glucosyltrehalose and maltooligosyltrehalose at the α-1,4 linkage between the second and the third glucose residues from the reducing end side to liberate α,α-trehalose.

29. The novel amylase claimed in any one of claims 25 to 28, wherein its molecular weight measured by SDS-polyacrylamide gel electrophoresis is 61,000 to 64,000, approximately.

30. The novel amylase claimed in any one of claims 25 to 29, wherein the amylase has the following physical and chemical properties:
(1) Optimum pH with in the range from 4.5 to 5.5;
(2) Optimum temperature within the range from 60 to 85° C.;
(3) pH Stability within the range from 4.0 to 10.0; and
(4) Thermostability which allow 100% enzymatic activity to remain even after exposure at 80° C. for 6 hours.

31. The novel amylase claimed in any one of claims 25 to 30, wherein the isoelectric point measured by isoelectric focusing is pH 4.3 to pH 5.4.

32. The novel amylase claimed in any one of claims 25 to 31, wherein its activity can be fully inhibited with 5 mM CuSO₄.

33. The novel amylase claimed in any one of claims 25 to 32, wherein the amylase is derived from an archaebacterium belonging to the order Sulfolobales.

34. The novel amylase claimed in claim 33, wherein the amylase is derived from an archaebacterium belonging to the genus Sulfolobus.

35. The novel amylase claimed in claim 34, wherein the archaebacterium belonging to the genus Sulfolobus is the Sulfolobus solfataricus strain KM1 (FERM BP-4626) or a variant thereof.

36. The novel amylase claimed in claim 34, wherein the archaebacterium belonging to the genus Sulfolobus is the Sulfolobus solfataricus strain DSM 5833 or a variant thereof.

37. The novel amylase claimed in claim 34, wherein the archaebacterium belonging to the genus Sulfolobus is the Sulfolobus acidocaldarius strain ATCC 33909 or a variant thereof.

38. A process for producing the amylase which is claimed in any one of claims 25 to 37, wherein said process comprises cultivating a bacterium having an ability of producing the amylase claimed in any one of claims 25 to 37 in a culture medium, and isolating and purifying said amylase from the culture according to an activity-measuring method in which the index is the activity of producing α,α-trehalose from a substrate trehaloseoligo-saccharide.

39. The process for producing amylase claimed in claim 38, wherein an archaebacterium belonging to the order Sulfolobales is cultivated.

40. The process for producing amylase claimed in claim 39, wherein an archaebacterium belonging to the genus Sulfolobus is cultivated.

41. The process for producing amylase claimed in claim 40, wherein the Sulfolobus solfataricus strain KM1 (FERM BP-4626) belonging to the genus Sulfolobus is cultivated.

42. The process for producing amylase claimed in claim 40, wherein the Sulfolobus solfataricus strain DSM 5833 belonging to the genus Sulfolobus is cultivated.

43. The process for producing amylase claimed in claim 40, wherein the Sulfolobus acidocaldarius strain ATCC 33909 belonging to the genus Sulfolobus is cultivated.

44. A process for producing α,α-trehalose, wherein the novel amylase claimed in any one of claim 25 to 37 is used in combination with a transferase which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage.

45. The process for producing α,α-trehalose claimed in claim 44, wherein said amylase and said transferase are put into a reaction at 60 to 80° C.

46. The process for producing α,α-trehalose claimed in claim 44 or 45, wherein the concentrations of said amylase and said transferase in the reaction mixture are 1.5 Units/ml or more and 0.1 Unit/ml or more, respectively.

47. The process for producing α,α-trehalose claimed in claim 44 or 45, wherein the concentrations of said amylase and said transferase in the reaction mixture are 1.5 Units/ml or more and 1 Unit/ml or more, respectively, and the ratio of the amylase concentration to the transferase concentration is 0.075 to 100.

48. The process for producing α,α-trehalose claimed in claim 47, wherein the concentrations of said amylase and said transferase in the reaction mixture are 15 Units/ml or more and 1 Unit/ml or more, respectively, and the ratio of the amylase concentration to the transferase concentration is 3 to 40.

49. The process for producing α,α-trehalose claimed in any one of claims 44 to 48, wherein the substrate is a saccharide composed of at least three sugar units, and at least three glucose residues from the reducing end of the substrate saccharide are α-1,4-linked.

50. The process for producing α,α-trehalose claimed in any one of claims 44 to 48, wherein the substrate is starch or a starch hydrolysate.

51. The process for producing α,α-trehalose claimed in claim 50, wherein said starch hydrolysate is produced from starch by acidolysis or enzymatic hydrolysis.

52. The process for producing α,α-trehalose claimed in claim 51, wherein said starch hydrolysate is obtained by using a debranching enzyme.

53. The process for producing α,α-trehalose claimed in claim 52, wherein said debranching enzyme is pullulanase or isoamylase.

54. The process for producing α,α-trehalose claimed in any one of claims 44 to 48, wherein the substrate is each or a mixture of maltooligosaccharides in which all the glucose residues are α-1,4-linked.

55. The process for producing α,α-trehalose claimed in claim 44 or 45, wherein a debranching enzyme is further used in combination.

56. The process for producing α,α-trehalose claimed in claim 55, wherein said debranching enzyme is pullulanase or isoamylase.

57. The process for producing α,α-trehalose claimed in claim 56, wherein pullulanase or isoamylase is used in combination one or more times in at least any one of the steps for producing α,α-trehalose.

58. The process for producing α,α-trehalose claimed in claim 57, wherein pullulanase or isoamylase is used in combination one or more times in at least any one of the early steps for producing α,α-trehalose.

59. The process for producing α,α-trehalose claimed in any one of claims 55 to 58, wherein the substrate is starch or a starch hydrolysate.

60. The process for producing α,α-trehalose claimed in claim 59, wherein said starch hydrolysate is produced from starch by acidolysis or enzymatic hydrolysis.

61. The process for producing α,α-trehalose claimed in claim 60, wherein said starch hydrolysate is obtained by using a debranching enzyme.

62. The process for producing α,α-trehalose claimed in claim 61, wherein said debranching enzyme is pullulanase or isoamylase.

63. The process for producing α,α-trehalose claimed in any one of claims 44 to 62, wherein an enzyme derived from an archaebacterium belonging to the order Sulfolobales is used as said transferase.

64. The process for producing α,α-trehalose claimed in claim 63, wherein an enzyme derived from an archaebacterium belonging to the genus Sulfolobus is used as said transferase.

65. The process for producing α,α-trehalose claimed in claim 63, wherein an enzyme derived from an archaebacterium belonging to the genus Acidianus is used as said transferase.

66. The process for producing α,α-trehalose claimed in claim 64, wherein an enzyme derived from the Sulfolobus solfataricus strain KM1 (FERM BP-4626) or a variant thereof is used as said transferase.

67. The process for producing α,α-trehalose claimed in claim 64, wherein an enzyme derived from the Sulfolobus solfataricus strain DSM 5833 or a variant thereof is used as said transferase.

68. The process for producing α,α-trehalose claimed in claim 64, wherein an enzyme derived from the Sulfolobus acidocaldarius strain ATCC 33909 or a variant thereof is used as said transferase.

69. The process for producing α,α-trehalose claimed in claim 65, wherein an enzyme derived from the Acidianus brierleyi strain DSM 1651 or a variant thereof is used as said transferase.

70. A DNA fragment comprising a DNA sequence which codes for the novel transferase claimed in claim 1.

71. The DNA fragment claimed in claim 70, wherein the optimum temperature for said novel transferase is 60 to 80° C.

72. The DNA fragment claimed in claim 70 or 71 expressed by the restriction map shown in FIG. 26.

73. The DNA fragment claimed in claim 70 or 71 expressed by the restriction map shown in FIG. 29.

74. A DNA fragment comprising a DNA sequence which codes for an amino acid sequence shown in Sequence No. 2 or an equivalent sequence thereof.

75. The DNA fragment claimed in claim 74 comprising a base sequence from the 335th base to the 2518th base of the base sequence shown in Sequence No. 1.

76. The DNA fragment claimed in claim 74 comprising a base sequence from the 1st to the 2578th base of the base sequence shown in Sequence No. 1.

77. A DNA fragment comprising a DNA sequence which codes for an amino acid sequence shown in Sequence No. 4 or an equivalent sequence thereof.

78. The DNA fragment claimed in claim 77 comprising a base sequence from the 816th base to the 2855th base of the base sequence shown in Sequence No. 3.

79. The DNA fragment claimed in claim 77 comprising a base sequence from the 1st base to the 3467th base of the base sequence shown in Sequence No. 3.

80. The DNA fragment claimed in any one of claims 70 to 79 derived from an archaebacterium belonging to the order Sulfolobales.

81. The DNA fragment claimed in claim 80 derived from an archaebacterium belonging to the genus Sulfolobus.

82. The DNA fragment claimed in claim 81 derived from the Sulfolobus solfataricus strain KM1.

83. The DNA fragment claimed in claim 81 derived from the Sulfolobus acidocaldarius strain ATCC 33909.

84. A DNA fragment which hybridizes with the base sequence from the 335th base to the 2518th base of the base sequence shown in Sequence No. 1 or a complementary sequence thereof at 40° C. under an ionic strength of 5×SSC, and which codes for a novel transferase acting on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage; and a DNA fragment which codes for the amino acid sequence encoded by the foregoing DNA fragment.

85. A DNA fragment which hybridizes with the base sequence from the 1880th base to the 2257th base of the base sequence shown in Sequence No. 1 or a complementary sequence thereof at 60° C. under an ionic strength of 6×SSPE, and which codes for a novel transferase acting on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage; and a DNA fragment which codes for the amino acid sequence encoded by the foregoing DNA fragment.

86. A polypeptide comprising an amino acid sequence shown in Sequence No. 2 or an equivalent sequence thereof.

87. A polypeptide comprising an amino acid sequence shown in Sequence No. 4 or an equivalent sequence thereof.

88. The polypeptide claimed in claim 86 or 87 which acts on a substrate saccharide, the substrate saccharide being composed of at least three, sugar units wherein at least three glucose residues from the reducing end are α-1,4-linked, so as to transfer the first α-1,4 linkage from the reducing end into an α-1,α-1 linkage.

89. The polypeptide claimed in any one of claims 86 to 88, wherein the optimum temperature for said activity is 60 to 80° C.

90. A recombinant DNA molecule comprising a DNA fragment claimed in any one of claims 70 to 85.

91. The recombinant DNA molecule claimed in claim 90, wherein said DNA fragment claimed in any one of claims 70 to 85 is combined in a plasmid vector.

92. The recombinant DNA molecule claimed in claim 90 or 91, wherein said molecule is the plasmid pKT22.

93. The recombinant DNA molecule claimed in claim 90 or 91, wherein said molecule is the plasmid p9TO1.

94. A host cell transformed with a recombinant DNA molecule claimed in any one of claim 90 to 93.

95. The host cell claimed in claim 94, wherein the host cell is a microorganism belonging to the genus Escherichia or Bacillus.

96. The host cell claimed in claim 95, wherein the host cell is the Escherichia coli strain JM109.

97. A process for producing a recombirant novel transferase which acts on a substrate saccharide, the substrate saccharide being composed of at least three sugar units wherein at least three glucose residues from the reducing end are α-1,4-linked, so as to transfer the first α-1,4 linkage from the reducing end into an α-1,α-1 linkage, wherein said process comprises cultivating a host cell claimed in any one of claims 94 to 96 to produce said recombinant novel transferase in the culture and collecting the transferase.

98. A process for producing a recombinant novel transferase which is encoded by a DNA fragment claimed in any one of claims 70 to 85 or which contains a polypeptide claimed in any one of claims 86 to 89, wherein said process comprises cultivating a host cell claimed in any one of claims 94 to 96 to produce said recombinant novel transferase in the culture and collecting the transferase.

99. A process for producing a trehaloseoligosaccharide in which at least three sugar units from the reducing end are glucose residues and the linkage between the first and second glucose residues from the reducing end side is α-1,α-1 while the linkage between the second and third glucose residues from the reducing end side is α-1,4, wherein the process comprises putting the recombinant novel transferase claimed in claim 97 or 98 into contact with a saccharide, the saccharide being composed of at least three sugar units wherein at least three glucose residues from the reducing end are α-1,4-linked.

100. A DNA fragment comprising a DNA sequence which codes for the novel amylase claimed in claim 25.

101. The DNA fragment claimed in claim 100 comprising a DNA sequence which codes for the novel amylase claimed in claim 26.

102. The DNA fragment claimed in claim 100 or 101 comprising a DNA sequence which codes for a novel amylase having an activity of endotype-hydrolyzing one or more of α-1,4 linkages in a sugar chain.

103. The DNA fragment claimed in any one of claims 100 to 102, wherein said novel amylase acts on a substrate trehaloseoligosaccharide so as to liberate α,α-trehalose by hydrolyzing the substrate at the α-1,4 linkage between the second and third glucose residues from the reducing end side.

104. A DNA fragment comprising a DNA sequence which codes for a novel amylase having the following principal activities:
(1) An activity of endotype-hydrolyzing one or more of α-1,4 glucoside linkages in a sugar chain;
(2) an activity of acting on a substrate saccharide, the substrate saccharide being composed of at least three sugar units wherein at least three sugar units from the reducing end are α-1,4-linked glucose residues, so as to liberate principally monosaccharides and/or disaccharides by hydrolyzing the substrate from the reducing end side; and
(3) an activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues from the reducing end side.

105. The DNA fragment claimed in any one of claims 100 to 104, wherein the optimum temperature for said novel amylase is 60 to 85° C.

106. The DNA fragment claimed in any one of claims 100 to 105 expressed by the restriction map shown in FIG. 34.

107. The DNA fragment claimed in any one of claims 100 to 105 expressed by the restriction map shown in FIG. 38.

108. A DNA fragment comprising a DNA sequence which codes for an amino acid sequence shown in Sequence No. 6 or an equivalent sequence thereof.

109. The DNA fragment claimed in claim 108 comprising the base sequence from the 642nd base to the 2315th base of the base sequence shown in Sequence No. 5.

110. The DNA fragment claimed in claim 108 comprising the base sequence from the 639th base to the 2315th base of the base sequence shown in Sequence No. 5.

111. The DNA fragment claimed in claim 108 comprising the base sequence from the 1st base to the 2691st base of the base sequence shown in Sequence No. 5.

112. A DNA fragment comprising a DNA sequence which codes for an amino acid sequence shown in Sequence No. 8 or an equivalent sequence thereof.

113. The DNA fragment claimed in claim 112 comprising the base sequence from the 1176th base to the 2843th base of the base sequence shown in Sequence No. 7.

114. The DNA fragment claimed in claim 112 comprising the base sequence from the 1st base to the 3600th base of the base sequence shown in Sequence No. 7.

115. The DNA fragment claimed in any one of claims 100 to 114, wherein said DNA fragment is derived from an archaebacterium belonging to the order Sulfolobales.

116. The DNA fragment claimed in claim 115, wherein said DNA fragment is derived from an archaebacterium belonging to the genus Sulfolobus.

117. The DNA fragment claimed in claim 116, wherein said DNA fragment is derived from the Sulfolobus solfataricus strain KM1.

118. The DNA fragment claimed in claim 116, wherein said DNA fragment is derived from the Sulfolobus acidocaldarius strain ATCC 33909 or a variant thereof

119. A DNA fragment which hybridizes with the base sequence from the 639th or 642nd base to the 2315th base of the base sequence shown in Sequence No. 5 or a complementary sequence thereof at 40° C. under an ionic strength of 5×SSC, and which codes for a novel amylase having an activity of acting on a substrate saccharide, the substrate saccharide being composed of at least three sugar units wherein at least three sugar units from the reducing end are glucose residues, so as to liberate principally monosaccharides and/or disaccharides by hydrolyzing the substrate from the reducing end side; and a DNA fragment which codes for the amino acid sequence encoded by the foregoing DNA fragment.

120. A DNA fragment which hybridizes with the base sequence from the 639th or 642nd base to the 2315th base of the base sequence shown in Sequence No. 5 or a complementary sequence thereof at 40° C. under an ionic strength of 5×SSC, and which codes for a novel amylase having a principal activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues; and a DNA fragment which codes for the amino acid sequence encoded by the foregoing DNA fragment.

121. A DNA fragment which hybridizes with the base sequence from the 1393th base to the 2121th base of the base sequence shown in Sequence No. 7 or a complementary sequence thereof at 60° C. under an ionic strength of 6×SSPE, and which codes for a novel amylase having an activity of acting on a substrate saccharide, the substrate saccharide being composed of at least three sugar units wherein at least three sugar units from the reducing end are glucose residues, so as to liberate principally monosaccharides and/or disaccharides by hydrolyzing the substrate from the reducing end side; and a DNA fragment which codes for the amino acid sequence encoded by the foregoing DNA fragment.

122. A DNA fragment which hybridizes with the base sequence from the 1393th base to the 2121th base of the base sequence shown in Sequence No. 7 or a complementary sequence thereof at 40° C. under an ionic strength of 6×SSPE, and which codes for a novel amylase having a principal activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues; and a DNA fragment which codes for the amino acid sequence encoded by the foregoing DNA fragment.

123. A polypeptide comprising an amino acid sequence shown in Sequence No. 6 or an equivalent sequence thereof.

124. A polypeptide comprising an amino acid sequence shown in Sequence No. 8 or an equivalent sequence thereof.

125. The polypeptide claimed in claim 123 further comprising Met at the N terminus.

126. The polypeptide claimed in any one of claims 123 to 125 which has an activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues.

127. The polypeptide claimed in any one of claims 123 to 125 which has the following principal activities:
(1) An activity of endotype-hydrolyzing one or more of α-1,4 glucoside linkages in a sugar chain;
(2) an activity of acting on a substrate saccharide, the substrate saccharide being composed of at least three sugar units wherein at least three sugar units from the reducing end are α-1,4-linked glucose residues, so as to liberate principally monosaccharide and/or disaccharide by hydrolyzing the substrate from the reducing end side; and
(3) an activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues.

128. The polypeptide claimed in any one of claims 123 to 127, wherein the optimum temperature for its action is 60 to 85° C.

129. A recombinant DNA molecule comprising a DNA fragment claimed in any one of claims 100 to 122.

130. The recombinant DNA molecule claimed in claim 129, wherein said DNA fragment claimed in any one of claims 100 to 122 is combined in a plasmid vector.

131. The recombinant DNA molecule claimed in claim 129 or 130, wherein said molecule is the plasmid pKA2.

132. The recombinant DNA molecule claimed in claim 129 or 130, wherein said molecule is the plasmid pO9A1.

133. A host cell transformed with a recombinant DNA molecule claimed in any one of claim 129 to 132.

134. The host cell claimed in claim 133, wherein the host cell is a microorganism belonging to the genus Escherichia or Bacillus.

135. The host cell claimed in claim 134, wherein the host cell is the Escherichia coli strain JM109.

136. A process for producing a recombinant novel amylase which has a principal activity of acting on a substrate saccharide, the substrate saccharide being composed of at least three sugar units wherein at least three sugar units from the reducing end side are glucose residues and the linkage between the first and second glucose residues from the reducing end side is α-1,α-1 while the linkage between the second and third glucose residues from the reducing end side is α-1,4, so as to liberate α,α-trehalose by hydrolyzing the α-1,4 linkage between the second and third glucose residues, wherein said process comprises cultivating a host cell claimed in any one of claims 133 to 135 to produce said recombinant novel amylase in the culture, and collecting the amylase.

137. A process for producing a recombinant novel amylase which is encoded by a DNA fragment claimed in any one of claims 100 to 122 or which contains a polypeptide claimed in any one of claims 123 to 128, wherein said process comprises cultivating a host cell claimed in any one of claims 133 to 135 to produce said recombinant novel amylase in the culture, and collecting the amylase.

138. A process for producing α,α-trehalose, wherein the process comprises putting the novel transferase claimed in any one of claim 1 to 13, or the recombinant novel transferase claimed in claim 97 or 98, and the recombinant novel amylase claimed in claim 136 or 137 into contact with a saccharide, the saccharide being composed of at least three sugar units wherein at least three glucose residues from the reducing end are α-1,4-linked.

139. A process for producing α,α-trehalose, wherein the process comprises putting the recombinant novel transferase claimed in claim 97 or 98, and the novel amylase claimed in any one of claim 25 to 37, or the recombinant novel amylase claimed in claim 136 or 137 into contact with a saccharide, the saccharide being composed of at least three sugar units wherein at least three glucose residues from the reducing end are α-1,4-linked.

140. The process claimed in claim 138 or 139, wherein the saccharide, which is composed of at least three sugar units wherein at least three glucose residues from the reducing end are α-1,4-linked, is starch or a starch hydrolysate.

141. The process claimed in claim 140, wherein said starch hydrolysate is produced from starch by acidolysis or enzymatic hydrolysis.

142. The process claimed in claim 140, wherein said starch hydrolysate is produced by hydrolyzing starch with a debranching enzyme.

143. The process claimed in claim 142, wherein said debranching enzyme is pullulanase or isoamylase.

144. The process claimed in claim 138 or 139, wherein the saccharide, which is composed of at least three sugar units wherein at least three glucose residues from the reducing end are α-1,4-linked, is each or a mixture of maltooligosaccharides in which all the glucose residues are α-1,4-linked.

145. The process claimed in any one of claims 138 to 144, wherein said process is performed at a temperature of 50 to 85° C.


SEQUENCE LISTING (63 sequences)

SEQ ID NO: 1 - DNA, 2578 bp, Sulfolobus solfataricus
  CDS (335)..(2518)
gcatgccattaaaagatgtaacattttacactccagacggtaaggaggttgatgagaaagcatggaattccccaacgcaaactgttattttcgtgttagaggggagcgtaatggatgagattaacatctatggagagagaattgcggatgattcattcttgataattcttaacgcaaatcccaataacgtaaaagtgaagttcccaaagggtaaatgggaactagttgttggttcttatttgagagagataaaaccagaagaaagaattgtagaaggtgagaaggaattggaaattgagggaagaacagcattagtttataggaggacagaactatgataataggcacatataggctgcaactcaataagaaattcactttttacgatataatagaaaatttggattattttaaagaattaggagtatcacacctatatctatctccaatacttaaggctagaccagggagcactcacggctacgatgtagtagatcatagtgaaattaatgaggaattaggaggagaagaggggtgctttaaactagttaaggaagctaagagtagaggtttagaaatcatacaagatatagtgccaaatcacatggcggtacatcatactaattggagacttatggatctgttaaagagttggaagaatagtaaatactataactattttgatcactacgatgatgacaagataatcctcccaatacttgaggacgagttggataccgttatagataagggattgataaaactacagaaggataatatagagtacagagggcttatattacctataaatgatgaaggagttgaattcttgaaaaggattaattgctttgataattcatgtttaaagaaagaggatataaagaaattactattaatacaatattatcagctaacttactggaagaaaggttatccaaactataggagatttttcgcagtaaatgatttgatagctgttagggtagaattggatgaagtatttagagagtcccatgagataattgctaagctaccagttgacggtttaagaattgaccacatagatggactatataaccctaaggagtatttagataagctaagacagttagtaggaaatgataagataatatacgtagagaagatattgtcaatcaacgagaaattaagagatgattggaaagtagatgggactactggatatgatttcttgaactacgttaatatgctattagtagatggaagtggtgaggaggagttaactaagttttatgagaatttcattggaaggaaaatcaatatagacgagttaataatacaaagtaaaaaattagttgcaaatcagttatttaaaggtgacattgaaagattaagcaagttactgaacgttaattacgattatttagtagattttctagcatgtatgaaaaaatacaggacttatttaccatatgaggatattaacggaataagggaatgcgataaggagggaaagttaaaagatgaaaagggaatcatgagactccaacaatacatgccagcaatcttcgctaagggctatgaggatactaccctcttcatctacaatagattaatttcccttaacgaggttgggagcgacctaagaagattcagtttaagcatcaaagactttcataactttaacctaagcagagtaaataccatatcaatgaacactctttccactcatgatactaaattcagtgaagacgttagagctagaatatcagtactatctgagataccaaaggagtgggaggagagggtaatatactggcatgatttgttaaggccaaatattgataaaaacgatgagtatagattttatcaaacacttgtgggaagttacgagggatttgataataaggagagaattaagaaccacatgattaaggtcataagagaagctaaggtacatacaacgtgggaaaatcctaatatagagtatgaaaagaaggttctgggtttcatagatgaagtgttcgagaacagtaattttagaaatgattttgaaaattttgaaaagaaaatagtttatttcggttatatgaaatcattaatcgcaacgacacttaggttcctttcgcccggtgtaccagatatttatcaaggaactgaagtttggagattcttacttacagacccagataacagaatgccggtggatttcaagaaactaaaggaattattaaataatttgactgaaaagaacttagaactctcagatccaagagtcaaaatgttatatgttaagaaattgctacagcttagaagagagtactcactaaacgattataaaccattgccctttggcttccaaaggggaaaagtagctgtccttttctcaccaatagtgactagggaggttaaagagaaaattagtataaggcaaaaaagcgttgattggatcagaaatgaggaaattagtagtggagaatacaatttaagtgagttgattgggaagcataaagtcgttatattaactgaaaaaagggagtgaactacctacatagatttattcttgaactactctggtcagaaatgtattacgcagatc

SEQ ID NO: 2 - protein, 728 aa, Sulfolobus solfataricus
MIIGTYRLQLNKKFTFYDIIENLDYFKELGVSHLYLSPILKARPGSTHGYDVVDHSEINEELGGEEGCFKLVKEAKSRGLEIIQDIVPNHMAVHHTNWRLMDLLKSWKNSKYYNYFDHYDDDKIILPILEDELDTVIDKGLIKLQKDNIEYRGLILPINDEGVEFLKRINCFDNSCLKKEDIKKLLLIQYYQLTYWKKGYPNYRRFFAVNDLIAVRVELDEVFRESHEIIAKLPVDGLRIDHIDGLYNPKEYLDKLRQLVGNDKIIYVEKILSINEKLRDDWKVDGTTGYDFLNYVNMLLVDGSGEEELTKFYENFIGRKINIDELIIQSKKLVANQLFKGDIERLSKLLNVNYDYLVDFLACMKKYRTYLPYEDINGIRECDKEGKLKDEKGIMRLQQYMPAIFAKGYEDTTLFIYNRLISLNEVGSDLRRFSLSIKDFHNFNLSRVNTISMNTLSTHDTKFSEDVRARISVLSEIPKEWEERVIYWHDLLRPNIDKNDEYRFYQTLVGSYEGFDNKERIKNHMIKVIREAKVHTTWENPNIEYEKKVLGFIDEVFENSNFRNDFENFEKKIVYFGYMKSLIATTLRFLSPGVPDIYQGTEVWRFLLTDPDNRMPVDFKKLKELLNNLTEKNLELSDPRVKMLYVKKLLQLRREYSLNDYKPLPFGFQRGKVAVLFSPIVTREVKEKISIRQKSVDWIRNEEISSGEYNLSELIGKHKVVILTEKRE

SEQ ID NO: 3 - DNA, 3467 bp, Sulfolobus acidocaldarius
  CDS (816)..(2855)
gctaataaactgaacaatgaggacggaatgaatgaaaattatagctggaattgtggagtagaaggagaaactaacgattctaatattctttattgtagagaaaaacaaagaagaaattttgtaataacattatttgttagccaaggtataccaatgatcttagggggagacgaaataggaagaacacaaaaaggcaacaataatgctttttgtcaggataatgagacaagttggtatgattggaaccttgatgaaaatcgtgtaaggtttcatgattttgtgaggagacttaccaatttttataaagctcatccgatatttaggagggctagatattttcagggtaagaagttacacggttccccattaaaggatgtgacgtggctaaaacctgacggcaatgaagttgatgattcagtgtggaaatctccaacaaatcatattatttatatattagagggaagtgctatcgatgaaataaattataatggagaaaggatagctgacgacacttttctaattattttgaatggagcaagtactaatcttaagataaaagtacctcatggaaaatgggagttagtgttacatccttatccacatgagccatctaacgataaaaagataatagaaaacaacaaagaagtagaaatagatggaaagactgcactaatttacaggaggatagagttccagtgatatcagcaacctacagattacagttaaataagaattttaattttggtgacgtaatcgataacctatggtattttaaggatttaggagtttcccatctctacctctctcctgtcttaatggcttcgccaggaagtaaccatgggtacgatgtaatagatcattcaaggataaacgatgaacttggaggagagaaagaatacaggagattaatagagacagctcatactattggattaggtattatacaggacatagtaccaaatcacatggctgtaaattctctaaattggcgactaatggatgtattaaaaatgggtaaaaagagtaaatattatacgtactttgactttttcccagaagatgataagatacgattacccatattaggagaagatttagatacagtgataagtaaaggtttattaaagatagtaaaagatggagatgaatatttcctagaatatttcaaatggaaacttcctctaacagaggttggaaatgatatatacgacactttacaaaaacagaattataccctaatgtcttggaaaaatcctcctagctatagacgattcttcgatgttaatactttaataggagtaaatgtcgaaaaagatcacgtatttcaagagtcccattcaaagatcttagatttagatgttgatggctatagaattgatcatattgatggattatatgatcctgagaaatatattaatgacctgaggtcaataattaaaaataaaataattattgtagaaaaaattctgggatttcaggaggaattaaaattaaattcagatggaactacaggatatgacttcttaaattactccaacttactgtttaattttaatcaagagataatggacagtatatatgagaatttcacagcggagaaaatatctataagtgaaagtataaagaaaataaaagcgcaaataattgatgagctatttagttatgaagttaaaagattagcatcacaactaggaattagctacgatatattgagagattacctttcttgtatagatgtgtacagaacttatgctaatcagattgtaaaagagtgtgataagaccaatgagatagaggaagcaaccaaaagaaatccagaggcttatactaaattacaacaatatatgccagcagtatacgctaaagcttatgaagatactttcctctttagatacaatagattaatatccataaatgaggttggaagcgatttacgatattataagatatcgcctgatcagtttcatgtatttaatcaaaaacgaagaggaaaaatcacactaaatgccactagcacacatgatactaagtttagtgaagatgtaaggatgaaaataagtgtattaagtgaatttcctgaagaatggaaaaataaggtcgaggaatggcatagtatcataaatccaaaggtatcaagaaatgatgaatatagatattatcaggttttagtgggaagtttttatgagggattctctaatgattttaaggagagaataaagcaacatatgataaaaagtgtcagagaagctaagataaatacctcatggagaaatcaaaataaagaatatgaaaatagagtaatggaattagtggaagaaacttttaccaataaggatttcattaaaagtttcatgaaatttgaaagtaagataagaaggatagggatgattaagagcttatccttggtcgcattaaaaattatgtcagccggtatacctgatttttatcagggaacagaaatatggcgatatttacttacagatccagataacagagtcccagtggattttaagaaattacacgaaatattagaaaaatccaaaaaatttgaaaaaaatatgttagagtctatggacgatggaagaattaagatgtatttaacatataagcttttatccctaagaaaacagttggctgaggattttttaaagggcgagtataagggattagatctagaagaaggactatgtgggtttattaggtttaacaaaattttggtaataataaaaaccaagggaagtgttaattacaaactgaaacttgaagagggagcaatttacacagatgtattgacaggagaagaaattaaaaaagaggtacagattaatgagctacctaggatactagttagaatgtaagttataataatccgatttttatgtgacaagatttacgcttacgaaaaggactgttaaatcaacttttatgtgaattatgaaacgtaaattataagtttcctgaggataaacatatatatctctatctctcattgatatcacatgagtattagattaaggggaagtaattcttacggacattcaggctggtttacagtatactgtagaatatgtaataggaaaataagaataggaacggacttagtctacaaatgccctaaatgtgaaaagaagtataacgcattcttctgtgaagcagatgctaggggattaaagaaaaagtgcccatactgtggtactgaacttgtcagtgcaatttaagactcaaatagaaggtaaaaatatttttatactgaataatgagttgttttacgctgatacggatatagttattcgaaatcaagattttattaagaaactcacctttacacaatataataagattgcctatattgacatggacatagaaacgacagaatttaagatattaagattagtagtgtgtaaaactagaataaatatttatgtttgcaacgtaattggtaaattgaaagaaactaattttgaaaa

SEQ ID NO: 4 - protein, 680 aa, Sulfolobus acidocaldarius
MASPGSNHGYDVIDHSRINDELGGEKEYRRLIETAHTIGLGIIQDIVPNHMAVNSLNWRLMDVLKMGKKSKYYTYFDFFPEDDKIRLPILGEDLDTVISKGLLKIVKDGDEYFLEYFKWKLPLTEVGNDIYDTLQKQNYTLMSWKNPPSYRRFFDVNTLIGVNVEKDHVFQESHSKILDLDVDGYRIDHIDGLYDPEKYINDLRSIIKNKIIIVEKILGFQEELKLNSDGTTGYDFLNYSNLLFNFNQEIMDSIYENFTAEKISISESIKKIKAQIIDELFSYEVKRLASQLGISYDILRDYLSCIDVYRTYANQIVKECDKTNEIEEATKRNPEAYTKLQQYMPAVYAKAYEDTFLFRYNRLISINEVGSDLRYYKISPDQFHVFNQKRRGKITLNATSTHDTKFSEDVRMKISVLSEFPEEWKNKVEEWHSIINPKVSRNDEYRYYQVLVGSFYEGFSNDFKERIKQHMIKSVREAKINTSWRNQNKEYENRVMELVEETFTNKDFIKSFMKFESKIRRIGMIKSLSLVALKIMSAGIPDFYQGTEIWRYLLTDPDNRVPVDFKKLHEILEKSKKFEKNMLESMDDGRIKMYLTYKLLSLRKQLAEDFLKGEYKGLDLEEGLCGFIRFNKILVIIKTKGSVNYKLKLEEGAIYTDVLTGEEIKKEVQINELPRILVRM

SEQ ID NO: 5 - DNA, 2691 bp, Sulfolobus solfataricus
  CDS (639)..(2315)
ctgcagtaactagcgctatcgaagacgttataaagagaaggataaatagagttccagtgagtctagaagacctttttgaataaggactttaatatcatttaaatttattttttggaacatgcagaggtaaacccatgaatgtcattttcgacgtattaaacgagatccatgggttttttggtgcattgtgggcgggagcagctctacttaactacttagttaagcctcaagataagaggcaatttgagagaatagggaaattcttcatgataaactcagtcattacagtaataactgggataataattttcgcctacatttacctagccccttatcaagggaatttatttctagtagcggcaattctacgttcaagccttgacattaggttaagggccttactaaacttaataggaggagcgtttgggttattggcttttggggcagggatagttataagcaataggataaggcttatggtacgtgttaaggaaggtgacgctacaatcctagagttgaggaatagtattgccaatttatctaaaattagtttaatcttcttattactttccttagccatgatgatacttgctggttccatagcacaagttataagttagagttgaaagaaaaatttaatgacgtttgcttataaaatagatggaaatgaggtaatctttaccttatgggcaccttatcaaaagagcgttaaactaaaggttctagagaagggactttacgaaatggaaagagatgaaaaaggttacttcaccattaccttaaacaacgtaaaggttagagataggtataaatacgttttagatgatgctagtgaaataccagatccagcatccagataccaaccagaaggtgtacatgggccttcacaaattatacaagaaagtaaagagttcaacaacgagacttttctgaagaaagaggacttgataatttatgaaatacacgtggggactttcactccagagggaacgtttgagggagtgataaggaaacttgactacttaaaggatttgggaattacggcaatagagataatgccaatagctcaatttcctgggaaaagggattggggttatgatggagtttatttatatgcagtacagaactcttacggagggccagaaggttttagaaagttagttgatgaagcgcacaagaaaggtttaggagttattttagacgtagtatacaaccacgttggaccagagggaaactatatggttaaattggggccatatttctcacagaaatacaaaacgccatggggattaacctttaactttgacgatgctgaaagcgatgaggttaggaagttcatcttagaaaacgttgagtactggattaaggaatataacgttgatgggtttagattagatgcggttcatgcaattattgacacttctcctaagcacatcttggaggaaatagctgacgttgtgcataagtataataggattgtcatagccgaaagtgatttaaacgatcctagagtcgttaatcccaaggaaaagtgtggatataatattgatgctcaatgggttgacgatttccatcattctattcacgcttacttaactggtgagaggcaaggctattatacggatttcggtaaccttgacgatatagttaaatcgtataaggacgttttcgtatatgatggtaagtactccaattttagaagaaaaactcacggagaaccagttggtgaactagacggatgcaatttcgtagtttatatacaaaatcacgatcaagtcggaaatagaggcaaaggtgaaagaataattaaattagtcgatagggaaagctacaagatcgctgcagccctttaccttctttccccctatattccaatgattttcatgggagaggaatacggtgaggaaaatcccttttatttcttttctgatttttcagattcaaaactgatacaaggtgtaagggaagggagaaaaaaggaaaacgggcaagatactgaccctcaagatgaatcaacttttaacgcttccaaactgagttggaagattgacgaggaaatcttttcattttacaagattttaataaaaatgagaaaggagttgagcatagcgtgtgataggagagtaaacgtcgtgaatggcgaaaattggttgatcatcaagggaagagaatacttttcactctacgttttctctaaatcatctattgaagttaagtacagtggaactttacttttgtcctcaaataattcattccctcagcatattgaagaaggtaaatatgagtttgataagggatttgctttatataaactttaggacaggagagtttaaaaatttctatgaatgattatactttagatgatgagtaaaagcaagatcgatgaggaagagaaaaggagaagagaagaagtcaaaaagttagtaatgctcttagcaatgttaagataatgtttttttaaactcaaataataataaataccatcatgtcaatattcttcagaactagagatagacctttacgtcccggagatccgtatccattaggttcaaattggatagaagatgaggatggcgtaaatttttccttgttctcagagaatgcagacaaagtggagttgattctttattcacaaacaaatcaaaagtatccaaaggagataatagaggttaagaatagaacgggggatcc

SEQ ID NO: 6 - protein, 558 aa, Sulfolobus solfataricus
TFAYKIDGNEVIFTLWAPYQKSVKLKVLEKGLYEMERDEKGYFTITLNNVKVRDRYKYVLDDASEIPDPASRYQPEGVHGPSQIIQESKEFNNETFLKKEDLIIYEIHVGTFTPEGTFEGVIRKLDYLKDLGITAIEIMPIAQFPGKRDWGYDGVYLYAVQNSYGGPEGFRKLVDEAHKKGLGVILDVVYNHVGPEGNYMVKLGPYFSQKYKTPWGLTFNFDDAESDEVRKFILENVEYWIKEYNVDGFRLDAVHAIIDTSPKHILEEIADVVHKYNRIVIAESDLNDPRVVNPKEKCGYNIDAQWVDDFHHSIHAYLTGERQGYYTDFGNLDDIVKSYKDVFVYDGKYSNFRRKTHGEPVGELDGCNFVVYIQNHDQVGNRGKGERIIKLVDRESYKIAAALYLLSPYIPMIFMGEEYGEENPFYFFSDFSDSKLIQGVREGRKKENGQDTDPQDESTFNASKLSWKIDEEIFSFYKILIKMRKELSIACDRRVNVVNGENWLIIKGREYFSLYVFSKSSIEVKYSGTLLLSSNNSFPQHIEEGKYEFDKGFALYKL

SEQ ID NO: 7 - DNA, 3600 bp, Sulfolobus acidocaldarius
  CDS (1176)..(2843)
attcgttttgagtcactcggcgtaggtctgtagtctttcttggcgagggctaataagttgagataatgcttgccaagaatcgaagaaggcgtcctgccctgcatgaaatcgattacctcggcactaactccgagctccgcgagtttagtagtcacgaatttgcgtacatatttcggcgctatccctttctcatgcaataaattcttcgcgtagttgtacgttatatcagtcttagctatagacgaaatgtgaaagacatagaacactttctttggccctctagtccagttgagcgtgtatacgtagaagccgtcctctttcacgttgttcttctcgtcatactcattgagaacctttacagcctccctaagccttataccgctctcaaggaggagcttgaagactagctctacctcaatacctctaacagcctccaaccacctccctatctcgtcagctcctggaaccttaagatcaacaccagactttttcgttttcagctttttccatgcctcaagatcccctttccacttgtagaacttcttccaggctaggatagagttcttagcattactagggggcttcttcagataattgatatactgcctgcaagtttcctcactggccattttcaaacaatattcataaaattcaattaattccttttccgtgagaccatttttgccctccctagaagtaagggagtttagggcaaatcccttactctcttcatcatttgaaagaggggttttaggggattcctcccctaaccagggctttggcccctgggaccagggttcgagtccctgcccggctacctttgaaaggttagggggatacaccctaataccccacttctatcttacaatttcaggtaagtctttactaggtcaactaaagcaccaacgtaagtctccttcgtcttaccaccttgactcttcttgataaagtaaacataatatcatccatagacttaccttattcttatattaccatatgattttattattttgtatttctattagataagtcccactcatagaacaaatgatggttttaacttatatactaaatactctaataactcaacaataataagaatttaatcagttctgataagtattttcactcgaaaacatttaaatatattaagacataatttctatttaaacagcatgttttcgttcggtggaaatattgaaaaaaataaaggtatctttaagttatgggcaccttatgttaatagtgttaagctgaagttaagcaaaaaacttattccaatggaaaaaaacgatgagggatttttcgaagtagaaatagacgatatcgaggaaaatttaacctattcttatattatagaagataagagagagatacctgatcccgcatcacgatatcaacctttaggagttcatgacaaatcacaacttataagaacagattatcagattcttgaccttggaaaagtaaaaatagaagatctaataatatatgaactccacgttggtactttttcccaagaaggaaatttcaaaggagtaatagaaaagttagattacctcaaggatctaggaatcacaggaattgaactgatgcctgtggcacaatttccagggaatagagattggggatacgatggtgtttttctatacgcagttcaaaatacttatggcggaccatgggaattggctaagctagtaaacgaggcacataaaaggggaatagccgtaattttggatgttgtatataatcatataggtcctgagggaaattaccttttaggattaggtccttatttttcagacagatataaaactccatggggattaacatttaattttgatgataggggatgtgatcaagttagaaaattcattttagaaaatgtcgagtattggtttaagacctttaaaatcgatggtctgagactggatgcagttcatgcaatttttgataattcgcctaagcatatcctccaagagatagctgaaaaagcccatcaattaggaaaatttgttattgctgaaagtgatttaaatgatccaaaaatagtaaaagatgattgtggatataaaatagatgctcaatgggttgacgatttccaccacgcagttcatgcattcataacaaaagaaaaagattattattaccaggattttggaaggatagaagatatagagaaaacttttaaagatgtttttgtttatgatggaaagtattctagatacagaggaagaactcatggtgctcctgtaggtgatcttccaccacgtaaatttgtagtcttcatacaaaatcacgatcaagtaggaaatagaggaaatggggaaagactttccatattaaccgataaaacgacataccttatggcagccacactatatatactctcaccgtatataccgctaatatttatgggcgaggaatattatgagacgaatccttttttcttcttctctgatttctcagatcccgtattaattaagggtgttagagaaggtagactaaaggaaaataatcaaatgatagatccacaatctgaggaagcgttcttaaagagtaaactttcatggaaaattgatgaggaagttttagattattataaacaactgataaatatcagaaagagatataataattgtaaaagggtaaaggaagttaggagagaagggaactgtattactttgatcatggaaaaaataggaataattgcatcgtttgatgatattgtaattaattctaaaattacaggtaatttacttataggcataggatttccgaaaaaattgaaaaaagatgaattaattaaggttaacagaggtgttggggtatatcaattagaatgaaagatcgaccattaaagcctggtgaaccttatcctttaggggcaacttggatagaggaagaagatggagttaattttgtactattctctgagaacgccacaaaagtagaactgttaacgtactctcagactagacaagatgagccaaaggaaataatagaacttagacagagaaccggagatctctggcatgtttttgtacctggtttaagaccaggtcagttgtatgggtacagggtgtatggtccatataaaccagaggaagggttaaggtttaatcctaataaagtactgatagatccttatgcaaaagctataaacggattattactatgggatgattcggtctttggatataaaattggagatcagaaccaggatctcagtttcgatgagagaaaagacgataaatttatacctaaaggggtcataataaatccttattttgattgggaggacgagcatttcttctttagaagaaagataccttttaaggatagtataatttatgagacacatataaaaggaataactaaattaaggcaagatttaccggagaacgttagaggcacttttttgggtttagcatcagatactatgattgattacctaaaagatttaggaattacaaccgttgagataatgcctattcagcaatttgtagatgagagattcattgtcgataaagggttaaagaactactggggttacaatccgataaattatttctctcctgaatgtagatactcaagctc

SEQ ID NO: 8 - protein, 556 aa, Sulfolobus acidocaldarius
MFSFGGNIEKNKGIFKLWAPYVNSVKLKLSKKLIPMEKNDEGFFEVEIDDIEENLTYSYIIEDKREIPDPASRYQPLGVHDKSQLIRTDYQILDLGKVKIEDLIIYELHVGTFSQEGNFKGVIEKLDYLKDLGITGIELMPVAQFPGNRDWGYDGVFLYAVQNTYGGPWELAKLVNEAHKRGIAVILDVVYNHIGPEGNYLLGLGPYFSDRYKTPWGLTFNFDDRGCDQVRKFILENVEYWFKTFKIDGLRLDAVHAIFDNSPKHILQEIAEKAHQLGKFVIAESDLNDPKIVKDDCGYKIDAQWVDDFHHAVHAFITKEKDYYYQDFGRIEDIEKTFKDVFVYDGKYSRYRGRTHGAPVGDLPPRKFVVFIQNHDQVGNRGNGERLSILTDKTTYLMAATLYILSPYIPLIFMGEEYYETNPFFFFSDFSDPVLIKGVREGRLKENNQMIDPQSEEAFLKSKLSWKIDEEVLDYYKQLINIRKRYNNCKRVKEVRREGNCITLIMEKIGIIASFDDIVINSKITGNLLIGIGFPKKLKKDELIKVNRGVGVYQLE

SEQ ID NO: 9 - protein, 6 aa, Sulfolobus solfataricus
VIREAK

SEQ ID NO: 10 - protein, 6 aa, Sulfolobus solfataricus
ISIRQK

SEQ ID NO: 11 - protein, 5 aa, Sulfolobus solfataricus
IIYVE

SEQ ID NO: 12 - protein, 5 aa, Sulfolobus solfataricus
MLYVK

SEQ ID NO: 13 - protein, 7 aa, Sulfolobus solfataricus
ILSINEK

SEQ ID NO: 14 - protein, 7 aa, Sulfolobus solfataricus
VVILTEK

SEQ ID NO: 15 - protein, 10 aa, Sulfolobus solfataricus
NLELSDPRVK

SEQ ID NO: 16 - protein, 12 aa, Sulfolobus solfataricus
MIIGTYRLQLNK

SEQ ID NO: 17 - protein, 9 aa, Sulfolobus solfataricus
VAVLFSPIV

SEQ ID NO: 18 - protein, 11 aa, Sulfolobus solfataricus
INIDELIIQSK

SEQ ID NO: 19 - protein, 12 aa, Sulfolobus solfataricus
ELGVSHLYLSPI

SEQ ID NO: 20 - protein, 7 aa, Sulfolobus solfataricus
DEVFRES

SEQ ID NO: 21 - protein, 4 aa, Sulfolobus solfataricus
DYFK

SEQ ID NO: 22 - protein, 7 aa, Sulfolobus solfataricus
DGLYNPK

SEQ ID NO: 23 - protein, 8 aa, Sulfolobus solfataricus
DINGIREC

SEQ ID NO: 24 - protein, 7 aa, Sulfolobus solfataricus
DFENFEK

SEQ ID NO: 25 - protein, 7 aa, Sulfolobus solfataricus
DLLRPNI

SEQ ID NO: 26 - protein, 5 aa, Sulfolobus solfataricus
DIIEN

SEQ ID NO: 27 - protein, 7 aa, Sulfolobus solfataricus
DNIEYRG

SEQ ID NO: 28 - DNA, 18 bp, Artificial Sequence
  Description of Artificial Sequence Primer
ytcwckraawacytcatc

SEQ ID NO: 29 - DNA, 20 bp, Artificial Sequence
  Description of Artificial Sequence Primer
gataayatwgartayagrgg

SEQ ID NO: 30 - protein, 8 aa, Sulfolobus solfataricus
RNPEAYTK

SEQ ID NO: 31 - protein, 9 aa, Sulfolobus solfataricus
DHVFQESHS

SEQ ID NO: 32 - protein, 8 aa, Sulfolobus solfataricus
ITLNATST

SEQ ID NO: 33 - protein, 6 aa, Sulfolobus solfataricus
IIIVEK

SEQ ID NO: 34 - protein, 11 aa, Sulfolobus solfataricus
LQQYMPAVYAK

SEQ ID NO: 35 - protein, 5 aa, Sulfolobus solfataricus
NMLES

SEQ ID NO: 36 - protein, 13 aa, Sulfolobus solfataricus
KISPDQFHVFNQK

SEQ ID NO: 37 - protein, 8 aa, Sulfolobus solfataricus
QLAEDFLK

SEQ ID NO: 38 - protein, 10 aa, Sulfolobus solfataricus
KILGFQEELK

SEQ ID NO: 39 - protein, 10 aa, Sulfolobus solfataricus
ISVLSEFPEE

SEQ ID NO: 40 - protein, 9 aa, Sulfolobus solfataricus
LKLEEGAIY

SEQ ID NO: 41 - protein, 8 aa, Sulfolobus solfataricus
EVQINELP

SEQ ID NO: 42 - protein, 5 aa, Sulfolobus solfataricus
DHSRI

SEQ ID NO: 43 - protein, 6 aa, Sulfolobus solfataricus
DLRYYK

SEQ ID NO: 44 - protein, 14 aa, Sulfolobus solfataricus
DVYRTYANQIVKEC

SEQ ID NO: 45 - protein, 10 aa, Sulfolobus solfataricus
TFAYKIDGNE

SEQ ID NO: 46 - protein, 7 aa, Sulfolobus solfataricus
LGPYFSQ

SEQ ID NO: 47 - protein, 7 aa, Sulfolobus solfataricus
DVFVYDG

SEQ ID NO: 48 - protein, 19 aa, Sulfolobus solfataricus
YNRIVIAESDLNDPRVVNP

SEQ ID NO: 49 - protein, 5 aa, Sulfolobus solfataricus
LDYLK

SEQ ID NO: 50 - protein, 17 aa, Sulfolobus solfataricus
KREIPDPASRYQPLGVH

SEQ ID NO: 51 - protein, 9 aa, Sulfolobus solfataricus
KDVFVYDGK

SEQ ID NO: 52 - protein, 9 aa, Sulfolobus solfataricus
HILQEIAEK

SEQ ID NO: 53 - protein, 10 aa, Sulfolobus solfataricus
KLWAPYVNSV

SEQ ID NO: 54 - protein, 7 aa, Sulfolobus solfataricus
MFSFGGN

SEQ ID NO: 55 - protein, 14 aa, Sulfolobus solfataricus
DYYYQDFGRIEDIE

SEQ ID NO: 56 - protein, 7 aa, Sulfolobus solfataricus
KIDAQWV

SEQ ID NO: 57 - DNA, 18 bp, Artificial Sequence
  Description of Artificial Sequence Primer
agcwagkagmtaycarcc

SEQ ID NO: 58 - DNA, 24 bp, Artificial Sequence
  Description of Artificial Sequence Primer
ytthccatcrtawacraawacatc

SEQ ID NO: 59 - protein, 6 aa, Sulfolobus solfataricus
DEFRES

SEQ ID NO: 60 - protein, 7 aa, Sulfolobus solfataricus
DNIEYRG

SEQ ID NO: 61 - protein, 7 aa, Sulfolobus solfataricus
PASRYQP

SEQ ID NO: 62 - protein, 8 aa, Sulfolobus solfataricus
DVFVYDGK

SEQ ID NO: 63 - protein, 559 aa, Sulfolobus solfataricus
MTFAYKIDGNEVIFTLWAPYQKSVKLKVLEKGLYEMERDEKGYFTITLNNVKVRDRYKYVLDDASEIPDPASRYQPEGVHGPSQIIQESKEFNNETFLKKEDLIIYEIHVGTFTPEGTFEGVIRKLDYLKDLGITAIEIMPIAQFPGKRDWGYDGVYLYAVQNSYGGPEGFRKLVDEAHKKGLGVILDVVYNHVGPEGNYMVKLGPYFSQKYKTPWGLTFNFDDAESDEVRKFILENVEYWIKEYNVDGFRLDAVHAIIDTSPKHILEEIADVVHKYNRIVIAESDLNDPRVVNPKEKCGYNIDAQWVDDFHHSIHAYLTGERQGYYTDFGNLDDIVKSYKDVFVYDGKYSNFRRKTHGEPVGELDGCNFVVYIQNHDQVGNRGKGERIIKLVDRESYKIAAALYLLSPYIPMIFMGEEYGEENPFYFFSDFSDSKLIQGVREGRKKENGQDTDPQDESTFNASKLSWKIDEEIFSFYKILIKMRKELSIACDRRVNVVNGENWLIIKGREYFSLYVFSKSSIEVKYSGTLLLSSNNSFPQHIEEGKYEFDKGFALYKL